(19) United States
(12) Patent Application Publication
(10) Pub. No.: US 2025/0115893 A1
(43) Pub. Date: Apr. 10, 2025

(54) HYDROGEL FOR IMMOBILIZATION OF ONE OR MORE ENZYME(S) AND METHOD FOR PREPARING THE SAME

(71) Applicants: Helmholtz Zentrum München - Deutsches Forschungszentrum für Gesundheit und Umwelt (GmbH), Neuherberg (DE); Gottfried Wilhelm Leibniz Universität Hannover, Hannover (DE)

(72) Inventors: Oliver PLETTENBURG, BURGDORF (DE); Christin AHLBRECHT, HANNOVER (DE); Samah AL MBARAK, HANNOVER (DE); Nick DIBBERT, HANNOVER (DE)

(21) Appl. No.: 18/700,903

(22) PCT Filed: Oct. 14, 2022
(86) PCT No.: PCT/EP2022/078635

§ 371 Date: Apr. 12, 2024

(30) Foreign Application Priority Data
Oct. 15, 2021 (EP) 21202802.1

(51) Int. Cl. C12N 11/10; C08B 37/00; C08B 37/02; C12N 9/04

(52) U.S. Cl. CPC C12N 11/10; C08B 37/0018; C08B 37/0021; C12N 9/0006; C12Y 101/03004

(57) ABSTRACT

The invention provides a method of preparing a biocompatible hydrogel, a hydrogel obtainable by that method, a biocompatible hydrogel for non-covalent immobilization of one or more enzyme(s), and a composition including any of those hydrogels. The invention further provides a method for encapsulating one or more enzyme(s) in a hydrogel as described herein and the use of any of said hydrogels for non-covalent immobilization of one or more enzyme(s) in the hydrogel or the use of any of said hydrogels in a biosensor. Additionally, the present invention provides a kit containing the composition or the hydrogel according to the present invention.

### TECHNICAL FIELD OF THE INVENTION

The present invention provides a method of preparing a biocompatible hydrogel, a hydrogel obtainable by said method, a biocompatible hydrogel for non-covalent immobilization of one or more enzyme(s) and a composition comprising any of said hydrogels according to the present invention. The present invention further provides a method for encapsulating one or more enzyme(s) in a hydrogel as described herein and the use of any of said hydrogels for non-covalent immobilization of one or more enzyme(s) in the hydrogel or the use of any of said hydrogels in a biosensor. Additionally, the present invention provides a kit comprising the composition or the hydrogel according to the present invention.

### BACKGROUND ART

Enzymes in solution usually have a limited lifetime, as they can quickly degrade at ambient temperature. Furthermore, they show limited tolerability towards organic solvents. The purpose of the present invention is inter alia to provide an effective method for enzyme stabilization. Enzyme encapsulation for various purposes usually requires covalent modification, leading to impaired activity. Furthermore, the bonds used for hydrogel formation may frequently not be stable, for example, when using Schiff bases, leading to premature degradation of the gel and subsequent leaching of the payload or may contain toxic and carcinogenic, e.g. hydrazones, functionalities.

Different methods have been developed to immobilize enzymes in a hydrogel network, e.g. under use of glutaraldehyde. However, for example, BSA mixtures provide limited protection of the enzymes and suffer from short lifetime and toxicity. Other methods of using wired enzymes require chemistry using toxic components and have limited application in implantable devices [1].

Other methods of the prior art use polymers, which are modified with furan/furan derivatives (methyl furan), reacting with poly(ethylene glycol) [2]. Other methods of the prior art uses, for the preparation of hydrogels, the formation of a Schiff base (between amino and aldehyde groups), leading to the formation of an imine linkage [3]. For example, Ma et al. uses the formation of a Schiff base (aldehyde and hydrazide groups) for building an injectable hydrogel [4].

Other possibilities comprise the chemo-specific ‘click reaction’ by an oxime crosslink. Though the oxime bound is more stable than hydrazine, this has the disadvantage that the hydrogel might be reversible in some biological environments [5].

Peng et al. instead provides stabilisation of collagen sponges by glutaraldehyde and uses vapour crosslinking [6]. Jia et al. [7] teaches to use an enzyme solution and a BSA stabilizer with chitosan in acetic acid, while the final crosslinking of the hydrogel was done with glutaraldehyde vapor.

However, all these described methods of the prior art have several disadvantages, which makes them not applicable for the encapsulation of enzymes. For example, the formation of a Schiff base has the problem of being reversible and hydrazones are undesired, because of their toxicity. Further, with the hydrogels provided in the prior art, reproducibility is limited, e.g. with regard to layer thickness. Further disadvantages are that coatings with imine/hydrazone are biodegradable, that products of degraded hydrazones (hydrazine) are toxic, that glutaraldehyde can't be spun, and that the quality control is insufficient, additionally, any vapour process is random and not reproducible [6].

Further, a major disadvantage of the prior art, for example, with regard to encapsulation of an enzyme within the hydrogel is that crosslinking by glutaraldehyde may result in covalent modifications of amino groups at the enzyme, which will lead to structural and conformational changes of the enzyme, possibly leading to undesired inactivation of the enzyme.

Also, the gelation process is starting immediately after mixing the individual components, leading to possible incomplete filling of form factors and to inhomogeneous hydrogels.

The present invention aims at and addresses these above described needs.

### SUMMARY OF THE INVENTION

The above-mentioned problems are solved by the subject-matter as defined in the claims and as defined herein.

The present invention describes a novel strategy to immobilize enzymes in a biocompatible hydrogel that does not require a covalent binding of one or more enzyme(s). The hydrogel according to the present invention provides a perfect stabilizing environment for the enzyme, resulting in a better lifetime and enzyme stability. The hydrogel according to the present invention protects the enzyme from biofouling and body immune response.

In a first aspect, the present invention provides a method of preparing a biocompatible hydrogel, comprising the following steps:
a) Providing a first polysaccharide and a second polysaccharide,
preferably wherein the first polysaccharide and/or the second polysaccharide being independently from each other selected from the group consisting of pullulan, alginate, hyaluronan and dextran,
with n being the number of the monomeric repeating unit of the first and/or second polysaccharide and with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—,
wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,

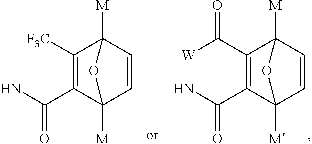

wherein Q is
wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In one aspect, the present invention provides a method of preparing a biocompatible hydrogel, comprising the following steps:
a) Providing a first polysaccharide and a second polysaccharide, preferably wherein the first polysaccharide and/or the second polysaccharide being independently from each other selected from the group consisting of pullulan, alginate, cellulose, hyaluronan, dextran, lichenin, lentinan and mixtures thereof, with n being the number of the monomeric repeating unit of the first and/or second polysaccharide and with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—,
wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O)ₜ—CH₂—CH₂-Q,
wherein Q is

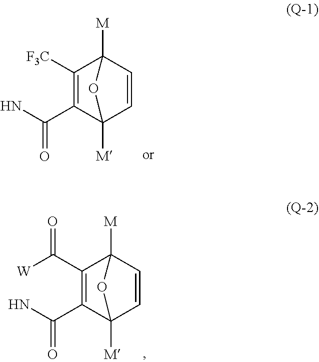

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In a further aspect, the present invention is directed to a hydrogel obtainable by a method of preparing a biocompatible hydrogel as described herein.

In one aspect, the present invention provides a biocompatible hydrogel comprising:
a crosslinked polymer comprising the following structure:
   a first polysaccharide and a second polysaccharide,
preferably wherein the first polysaccharide and/or the second polysaccharide being independently selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

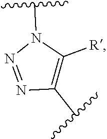

wherein R′ is selected from the group consisting of —CF₃, —C(O)—OMe, —C(O)—OEt, —C(O)—OH, —C(O)—NH₂ and —C(O)—NHMe, for non-covalent immobilization of one or more enzyme(s).

The present invention further provides a composition comprising the hydrogel according to any aspect of the present invention as described herein.

Further is provided by the present invention a method for encapsulating one or more enzyme(s) in a hydrogel according to any aspect of the present invention as described herein.

The present invention further provides the use of a hydrogel according to any aspect as described herein
a) for non-covalent immobilization of one or more enzyme(s) in the hydrogel,
b) in a biosensor.

The present invention further provides a kit comprising the composition or the hydrogel according to any aspect of the present invention as described herein.

### BRIEF DESCRIPTION OF THE DRAWINGS

FIG. 1 shows an exemplary structure of a biocompatible hydrogel according to the present invention.

FIG. 2 shows an exemplary basic principle of the gelation to form the biocompatible hydrogel according to the method of the present invention.

FIG. 3 shows the micro-morphology, wherein the 3D-hydrogel network was analysed by scanning electron microscopy (SEM) with forced ion beam (FIB). FIG. 3A shows pullulan hydrogels polymerized in PBS buffer and FIGS. 3B and 3C show pullulan hydrogels polymerized in dH₂O.

FIG. 4 shows the surface layer, the roughness and the pores in the outer surface analysed by atomic force microscopy (AFM). With increasing degree of modification, the layer gets denser and contains fewer microcavities and pores.

FIG. 5 shows the degree of substitution in a pullulan biopolymer with different amounts of introduced carboxymethyl groups. It displays different fingerprint regions in the FT-IR spectrum to characterize the carboxymethylation degree of pullulan (for PCM1, PCM3 and PCM5). The intensity of the bands between 1600-1000 cm⁻¹ increases with the substitution degree.

FIG. 6 shows the increase of the introduced carboxymethyl-groups in a biopolymer by ¹H-NMR spectroscopy. The intensity of the signals of the anomeric protons of the polysaccharide (square) decreases with higher degrees of modification, while the signals of the polysaccharide backbone broadens (dashed) and the intensity of the signals of the introduced carboxymethyl-group(s) (dotted) increases.

FIG. 7 shows the stepwise modification process of polysaccharides (e.g. pullulan) by ¹H-NMR spectroscopy. It compares the carboxymethylated with the unmodified polysaccharide. In this context, the intensity of the anomeric proton signals decreases (see c and d). The signals of the oxanorbornadiene linker unit introduced by the copper-free cycloaddition into the polysaccharide-chain are visible in the NMR-spectra of the ‘PCM3 linker’ (see a and b).

FIG. 8 shows the direct correlation of the degree of substitution of the polysaccharide with the introduced oxanorbornadiene linker units by ¹H-NMR spectroscopy. c and d show the decrease of the intensity of the anomeric proton signals with increasing degree of substitution. a and b show the increase of the intensity of the introduced linker signals due to the increasing number of carboxymethyl-groups in the polysaccharide.

FIG. 9 shows the increase in the degree of substitution due to the repetitive carboxymethylation reaction, measured by conductive titration. Data is given for a low molecular weight dextran (10 kDa) and molecular weight pullulan (100 kDa).

FIG. 10 shows IR-spectra of five different batches of a five times carboxymethylated pullulan strand (PCM5), which contains an azide linker unit ((—NH—(CH₂CH₂O)ₛ—CH₂CH₂N₃ with s=3)). Data is given for molecular weight pullulan (100 kDa).

FIG. 11 shows the time course of the storage module in a rheology measurement of three modified pullulan hydrogels with different carboxymethylation degrees.

FIG. 12 shows the increase of the intensity of the signal in a ¹⁹F-NMR spectrum of an introduced linker in a pullulan strand due to the increase of the degree of substitution and the number of introduced linker units in relation to an internal standard.

FIGS. 13A and 13B shows the results for the determined swelling rates for pullulan hydrogels with low and highly modified pullulan (PCM1 and PCM8) obtained according to the procedure of Example 6. W=dH₂O; P3=PBS buffer; pH 3.1, P7=PBS buffer; pH 7.4; W—P3 means gelation in dH₂O and after lyophilization swelling in PBS buffer, pH 3.1.

FIG. 14 shows enzyme saturation curves for glucose oxidase (50 mU/mL) in solution (FIG. 14A) and two curves of pullulan hydrogel samples with immobilized enzyme, which are low (PCM2, FIG. 14B) and highly modified (PCM8, FIG. 14C).

FIG. 15 shows the longevity of glucose oxidase (25 mU/mL) in hydrogels, in detail after immobilization in PCM9 vs. the enzyme in solution. The kinetics of glucose oxidase in solution and immobilized in hydrogel is shown after 10 days (see FIG. 15A) and 43 days (see FIG. 15B).

FIG. 16 shows the longevity of glucose oxidase (100 mU/mL) in hydrogels, in detail after immobilization in PCM5 vs. the enzyme in solution. The kinetics of glucose oxidase in solution and immobilized in hydrogel is shown after 10 days (see FIG. 16A) and 15 days (see FIG. 16B).

FIG. 17 shows longevity data for the enzyme uricase (100 mU/mL), in detail, the kinetics of uricase immobilized in hydrogel (PCM1) over time.

FIG. 18 shows the degree of substitution in a dextran biopolymer (10 kDa) with different amounts of introduced carboxymethyl groups. It displays different fingerprint regions in the FT-IR spectrum to characterize the different carboxymethylation degree (for CM1, CM3 and CM5). The intensity of the bands between 1600-1000 cm-1 increases with the substitution degree.

FIG. 19 shows the differences between a native lentinan and a carboxymethylated lentinan (CM1). It visualizes different fingerprint regions in the FT-IR spectrum to verify the carboxymethylation degree (lentinan and lentinan CM1). The intensity of the bands between 1600-1000 cm-1 increases with the substitution degree.

FIG. 20 shows the differences between hyaluronan and hyaluronan with linker units (azide unit and an oxanorbornadiene unit). The wavenumbers of the bands in the FT-IR spectra vary between 1600-1000 cm-1 due to the differently employed linker units.

FIG. 21 shows a ¹H-NMR spectrum of a 5-times carboxymethylated pullulan biopolymer with an azide linker unit (—NH—(CH₂)ᵣ—N₃ with r=6).

FIG. 22 shows a ¹H-NMR spectrum of a 5-times carboxymethylated pullulan with an oxanorbornadiene unit (—NH—(CH₂)ᵣ-Q with r=6, Q=Q−1 (CF₃) and M/M′=H).

FIG. 23 shows a ¹H-NMR spectrum of a 5-times carboxymethylated pullulan with an azide unit (—NH—(CH₂CH₂O)ₛ—CH₂CH₂N₃ with s=8).

FIG. 24 shows a ¹H-NMR spectrum of hyaluronan (A) in comparison to hyaluronan with an oxanorbornadiene unit (—NH—(CH₂)ᵣ-Q with r=2, Q=Q−1 (CF₃) and M/M′=H) (B), and hyaluronan with an azide unit (—NH—(CH₂CH₂O)ₛ—CH₂CH₂N₃ with s=3) (C).

FIG. 25 shows ¹H-NMR spectra of dextran (10 kDa) with different amounts of introduced carboxymethyl groups.

FIG. 26 shows the viscosities of various biopolymers at a shear rate of 100 s⁻¹. Different polysaccharides (A), pullulan with various carboxymethyl groups (B), differences in viscosity due to pullulan modification (native pullulan, 6-times carboxymethylated pullulan and pullulan Mix (Mix: pullulan with azide linker and pullulan with oxanorbornadiene unit, before gelation) (C), differences in viscosity due to alginate modification (native alginate and alginate Mix (Mix: alginate with azide linker and alginate with oxanorbornadiene unit, before gelation) (D).

FIG. 27 shows enzyme kinetics of immobilized GOx in pullulan hydrogels (PCM1 and PCM9) after different washing steps.

FIG. 28 shows enzyme kinetics of immobilized UOx in non-washed and washed pullulan hydrogels (PCM1) (A) and in the supernatant (B).

FIG. 29 shows enzyme kinetics of immobilized UOx in a pullulan hydrogel (PCM5).

FIG. 30 shows enzyme kinetics of immobilized GOx in various hydrogels. Dextran hydrogels (A), hyaluronan and alginate hydrogels (B), alginate-pullulan hydrogels (C) and pullulan hydrogels consisting of these two linker units before gelation: 5-times carboxymethylated pullulan with an oxanorbornadiene unit (—NH—(CH₂)ᵣ-Q with r=6, Q=Q−1 (CF₃) and M/M′=H) and 5-times carboxymethylated pullulan with an azide unit (—NH—(CH₂CH₂O)ₛ—CH₂CH₂N₃ with s=8) (D).

### DETAILED DESCRIPTION OF THE INVENTION

The invention provides in a first aspect a method of preparing a biocompatible hydrogel, comprising the following steps:
a) Providing a first polysaccharide and a second polysaccharide,
preferably wherein the first polysaccharide and/or the second polysaccharide being independently from each other selected from the group consisting of pullulan, alginate, hyaluronan and dextran,
with n being the number of the monomeric repeating unit of the first and/or second polysaccharide, and
with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the first polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

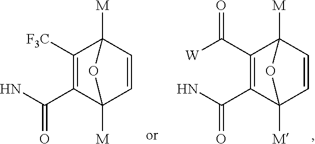

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

The invention provides in one aspect a method of preparing a biocompatible hydrogel, comprising the following steps:
a) Providing a first polysaccharide and a second polysaccharide, preferably wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the first polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

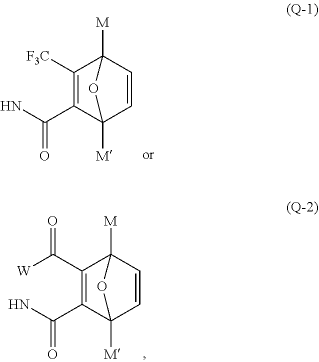

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In one embodiment, the method of preparing a biocompatible hydrogel, comprises the following steps:
a) Providing a first polysaccharide and a second polysaccharide,
preferably wherein the first polysaccharide and/or the second polysaccharide being independently from each other selected from the group consisting of pullulan, alginate, cellulose, hyaluronan, dextran, lichenin, lentinan and mixtures thereof, with n being the number of the monomeric repeating unit of the first and/or second polysaccharide, and
with n being an integer from 10 to 10000,
b) carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q, wherein Q is

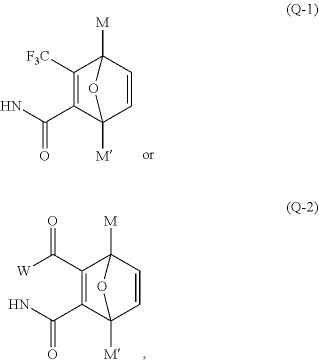

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In one embodiment, the method of preparing a biocompatible hydrogel, comprises the following steps:
a) Providing a first polysaccharide and a second polysaccharide, preferably wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃,
and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

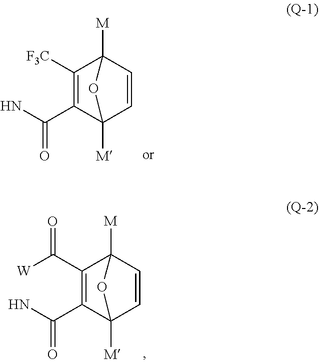

wherein M, M′=H or Me, and
wherein W=OMe, OEt, OH, NH₂ or NHMe,
d) addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In one embodiment, the method of preparing a biocompatible hydrogel, comprises the following steps:
a) Providing a first polysaccharide and a second polysaccharide, preferably wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
b) carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

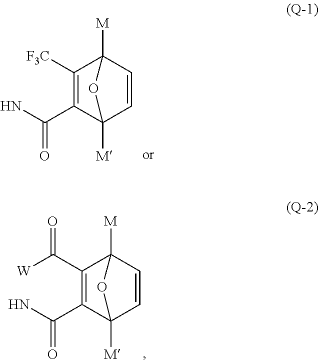

wherein M, M′=H or Me, and
wherein W=OMe, OEt, OH, NH₂ or NHMe,
d) addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

In one embodiment, the method of preparing a biocompatible hydrogel, comprises the following steps:
a) Providing a first polysaccharide and a second polysaccharide,
preferably wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

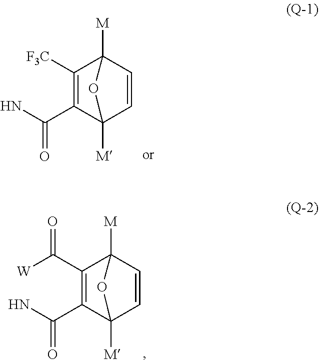

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 30° C. to 60° C., preferably 40° C., for at least 1 hour, preferably for 1 to 10 hours.

In one embodiment, the method of preparing a biocompatible hydrogel according to the present invention, comprises the following steps:
a) Providing a first polysaccharide and a second polysaccharide,
preferably wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the first polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

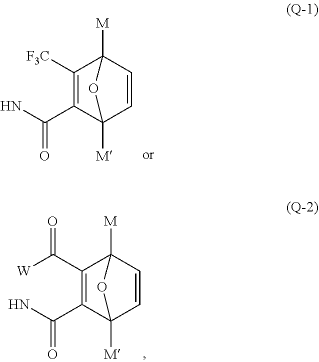

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 30° C. to 60° C. for 1 to 10 hours.

As used herein and in the context of the present invention, the term “biocompatible” means, especially in connection with a hydrogel, that the respective material being called or assessed as being biocompatible has the quality of not having toxic or injurious effects on biological systems, that it has the ability to perform its desired function without eliciting any undesirable local or systemic effects in the recipient, but generating the most appropriate beneficial response in that specific situation, or the ability to exist in harmony with tissue without causing deleterious changes. Preferable properties of biocompatible materials are reduced inflammation and immunological response and/or low/limited fibrotic encapsulation.

The term “hydrogel”, as used herein and in the context of the present invention, is a term being well known to a person skilled in the art and includes any network of covalently crosslinked polymer chains that are hydrophilic. It usually builds up a three-dimensional solid, consisting of hydrophilic polymer chains, being held together by specific crosslinkers. Because of the inherent crosslinkers, the structural integrity of the hydrogel network does not dissolve in water. Hydrogels are highly absorbent (they can contain over 90% water) natural or synthetic polymeric networks.

The “first polysaccharide” and the “second polysaccharide” as used within the context of the present invention may be any polysaccharide known to a person skilled in the art. However, it is preferred that the first polysaccharide and/or the second polysaccharide may be independently from each other selected from the group consisting of pullulan, alginate, cellulose, hyaluronan, dextran, lichenin, lentinan and mixtures thereof, more preferably from the group consisting of pullulan, alginate, hyaluronan and dextran. In this connection, the respective “monomeric repeating unit” for each of these exemplary examples for the first and/or second polysaccharide may be defined as follows herein below:

Pullulan is a polysaccharide polymer consisting of maltotriose units. Three glucose units of maltotriose are connected by an α-1,4-glycosidic bond, whereas consecutive maltotriose units are connected to each other by an α-1,6-glycosidic bond. Pullulan may be produced from starch by the fungus Aureobasidium pullulans. It may be mainly used by the cell to resist desiccation and predation. The presence of this polysaccharide also facilitates diffusion of molecules both into and out of the cell. In the context of the present invention, the respective monomeric repeating unit for pullulan has the structure of

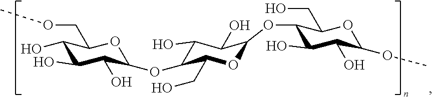

with n being the number of said monomeric repeating unit of pullulan, and with n being an integer from 10 to 10000. In the context of the present invention, the carboxymethylation degree of pullulan is presented by the expression “PCM” followed by a number (e.g. PCM1, PCM3 and PCM5). This number characterizes the respective carboxymethylation degree, meaning the carboxymethyl-groups introduced into pullulan by applying the designated number of repetitive carboxymethylation reaction cycles. As used herein, CM1, CM2, CM3, etc. describe the respective carboxymethylation degree of a polysaccharide in general (without specifically referring to pullulan).

Alginic acid, also called algin, is a polysaccharide distributed widely in the cell walls of brown algae that is hydrophilic and forms a viscous gum, when being hydrated. Alginic acid is a linear copolymer with homopolymeric blocks of (1-4)-linked β-D-mannuronate (M) and its C-5 epimer α-L-guluronate (G) residues, respectively, are covalently linked together in different sequences or blocks. The monomers may appear in homopolymeric blocks of consecutive G-residues (G-blocks), consecutive M-residues (M-blocks) or alternating M- and G-residues (MG-blocks). With metals, such as sodium and calcium, its salts are known as alginates. In the context of the present invention, the respective monomeric repeating unit for alginate has the structure of

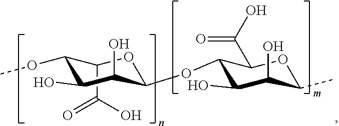

with n and m being the number of the monomeric repeating unit of alginate, and with n and m being each independently from each other an integer in the range from 10 to 10000.

Hyaluronic acid (abbreviated HA; conjugate base: hyaluronate), also called hyaluronan, is an anionic, non-sulfated glycosaminoglycan distributed widely throughout connective, epithelial and neural tissues. It is unique among glycosaminoglycans in that it is non-sulfated, forms in the plasma membrane instead of the Golgi apparatus and can be very large. Hyaluronic acid is a polymer of disaccharides, themselves composed of D-glucuronic acid and N-acetyl-D-glucosamine, linked via alternating β-(1→4) and β-(1→3) glycosidic bonds. In the context of the present invention, the respective monomeric repeating unit for hyaluronan has the structure of

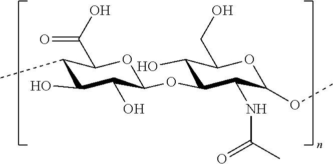

with n being the number of said monomeric repeating unit of hyaluronan, and with n being an integer from 10 to 10000.

Dextran is a complex branched glucan (polysaccharide derived from the condensation of glucose). IUPAC defines dextrans as “branched poly-α-D-glucosides of microbial origin having glycosidic bonds predominantly C-1→C-6”. Dextran chains are of varying lengths (from 3 to 2000 kilodaltons). The polymer main chain consists of α-1,6-glycosidic linkages between glucose monomers, with random branches from α-1,3-linkages. This characteristic branching distinguishes a dextran from a dextrin, which is a straight chain glucose polymer tethered by α-1,4- or α-1,6-linkages. In the context of the present invention, the respective monomeric repeating unit for dextran has the structure of

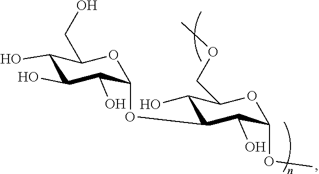

with n being the number of said monomeric repeating unit of dextran, and with n being an integer from 10 to 10000.

The first and/or second polysaccharide of the method of the present invention may be optionally carboxymethylated in step b), wherein at least one OH-group of the first and/or second polysaccharide as defined herein above may be carboxymethylated. In one embodiment of the method of the present invention, the carboxymethylation is preferably carried out, when the first and/or second polysaccharide is/are pullulan or dextran.

Functionalization of the first polysaccharide as defined above in step c) of the method of the present invention may be with one or more linker unit(s) of the structure -A-X, when the monomeric repeating unit of the first polysaccharide not comprises a carboxylic acid residue, or functionalization of the first polysaccharide as defined above in step c) of the method of the present invention may be with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15.

Functionalization of the second polysaccharide as defined above in step c) of the method of the present invention may be with one or more linker unit(s) of the structure -A′-Y, when the monomeric repeating unit of the second polysaccharide not comprises a carboxylic acid residue, or functionalization of the second polysaccharide as defined above in step c) of the method of the present invention may be with one or more linker unit(s) of the structure —Y, when the monomeric repeating unit of the second polysaccharide comprises a carboxylic acid residue, wherein A′ is —(CH₂)_{d}—C(O)— or —C(O)—NH—, wherein d is an integer from 1 to 3, wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

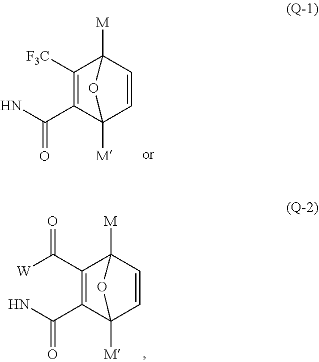

wherein M, M′=H or Me, and
wherein W=OMe, OEt, OH, NH₂ or NHMe.

As used in the present invention, Q may be

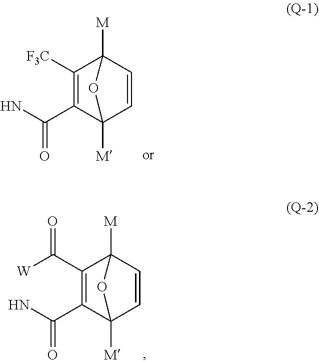

wherein M, M′=H or Me, and
wherein W=OMe, OEt, OH, NH₂ or NHMe. If, for the structural formula

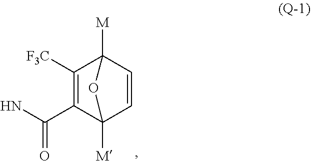

the structural formula

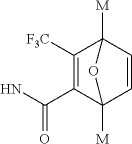

is used elsewherein herein, this does not mean for the latter mentioned structural formula that both M have to be the same (each H or each Me). Rather, the present invention also comprises that, in one embodiment both M can be H, in one embodiment both M can be Me, and in one embodiment one M can be H and the other M can be Me (independent from the position of M, two possibilities for one M being H and the other M being Me). Thus, in the context of the present invention, the two structural formulas

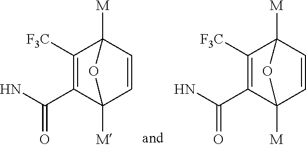

can be used interchangeably herein.

Moreover, in the context of the present invention, it is absolutely clear for the person skilled in the art, with regard to the functionalization of the second polysaccharide with one or more linker unit(s) of the structure -A′-Y,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is

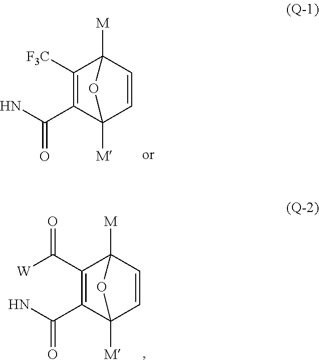

wherein M, M′=H or Me, and
wherein W=OMe, OEt, OH, NH₂ or NHMe, that the linking of Q within —NH—(CH₂)ᵣ-Q, —NH—(CH₂CH₂O)ₛ—CH₂CH₂Q or —NH—(CH₂—CH₂—C(O))ₜ—CH₂—CH₂-Q, is via the dashed line given in the structural formulas

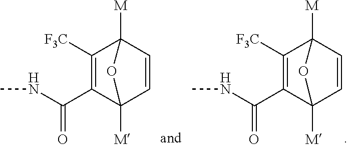

As used herein and in the context of the present invention, the term “functionalization” or “functionalized” means in general the addition of specific functional groups to afford the compound new, desirable properties, e.g. in the present invention, the addition of a linker unit or linker units as defined above to the existent polysaccharide structure.

In one embodiment, in step d) of the method of the present invention, optionally the addition of one or more enzyme(s) to the mixture formed by the steps a)-c) may be carried out. In this embodiment, any enzyme is in principle possible for the method of the present invention.

In step e) of the method of the present invention, the incubation of the mixture formed by steps a)-d) is carried out in an aqueous medium at a temperature being in the range from 25° C. to 70° C. for at least one hour. It is more preferred that in step e) of the method of the present invention, the incubation of the mixture formed by steps a)-d) is carried out in an aqueous medium at a temperature being in the range from 25° C. to 70° C. for 1-15 hours. It is even more preferred that in step e) of the method of the present invention, the incubation of the mixture formed by steps a)-d) is carried out in an aqueous medium at a temperature being in the range from 25° C. to 70° C. for 1-10 hours.

It is preferred that in step e) of the method of the present invention, the incubation of the mixture formed by steps a)-d) is carried out in an aqueous medium at a temperature being in the range from 30° C. to 60° C. for at least one hour. It is more preferred that in step e) of the method of the present invention, the incubation of the mixture formed by steps a)-d) is carried out in an aqueous medium at a temperature being in the range from 30° C. to 60° C. for 1-15 hours. It is even more preferred that in step e) of the method of the present invention, the incubation of the mixture formed by steps a)-d) is carried out in an aqueous medium at a temperature being in the range from 30° C. to 60° C. for 1-10 hours.

It is further preferred that in step e) of the method of the present invention, the incubation of the mixture formed by steps a)-d) is carried out in an aqueous medium at a temperature being in the range from 35° C. to 50° C. for at least one hour. It is more preferred that in step e) of the method of the present invention, the incubation of the mixture formed by steps a)-d) is carried out in an aqueous medium at a temperature being in the range from 35° C. to 50° C. for 1-15 hours. It is even more preferred that in step e) of the method of the present invention, the incubation of the mixture formed by steps a)-d) is carried out in an aqueous medium at a temperature being in the range from 35° C. to 50° C. for 1-10 hours.

It is further preferred that in step e) of the method of the present invention, the incubation of the mixture formed by steps a)-d) is carried out in an aqueous medium at a temperature being in the range from 35° C. to 45° C. for at least one hour. It is more preferred that in step e) of the method of the present invention, the incubation of the mixture formed by steps a)-d) is carried out in an aqueous medium at a temperature being in the range from 35° C. to 45° C. for 1-15 hours. It is even more preferred that in step e) of the method of the present invention, the incubation of the mixture formed by steps a)-d) is carried out in an aqueous medium at a temperature being in the range from 35° C. to 45° C. for 1-10 hours.

It is more preferred that in step e) of the method of the present invention, the incubation of the mixture formed by steps a)-d) is carried out in an aqueous medium at a temperature of about 40° C. for at least one hour. It is even more preferred that in step e) of the method of the present invention, the incubation of the mixture formed by steps a)-d) is carried out in an aqueous medium at a temperature of about 40° C. for 1-15 hours. It is even more preferred that in step e) of the method of the present invention, the incubation of the mixture formed by steps a)-d) is carried out in an aqueous medium at a temperature of about 40° C. for 1-10 hours.

It is further preferred that in step e) of the method of the present invention, the incubation of the mixture formed by steps a)-d) is carried out in an aqueous medium at a temperature being in the range from 30° C. to 60° C. for 4 to 10 hours. It is further preferred that in step e) of the method of the present invention, the incubation of the mixture formed by steps a)-d) is carried out in an aqueous medium at a temperature being in the range from 35° C. to 50° C. for 4 to 10 hours. It is further preferred that in step e) of the method of the present invention, the incubation of the mixture formed by steps a)-d) is carried out in an aqueous medium at a temperature being in the range from 35° C. to 45° C. for 4 to 10 hours. It is more preferred that in step e) of the method of the present invention, the incubation of the mixture formed by steps a)-d) is carried out in an aqueous medium at a temperature of about 40° C. for 4 to 10 hours.

It is further preferred that in step e) of the method of the present invention, the incubation of the mixture formed by steps a)-d) is carried out in an aqueous medium at a temperature being in the range from 30° C. to 60° C. for 4 to 6 hours. It is further preferred that in step e) of the method of the present invention, the incubation of the mixture formed by steps a)-d) is carried out in an aqueous medium at a temperature being in the range from 35° C. to 50° C. for 4 to 6 hours. It is further preferred that in step e) of the method of the present invention, the incubation of the mixture formed by steps a)-d) is carried out in an aqueous medium at a temperature being in the range from 35° C. to 45° C. for 4 to 6 hours. It is more preferred that in step e) of the method of the present invention, the incubation of the mixture formed by steps a)-d) is carried out in an aqueous medium at a temperature of about 40° C. for 4 to 6 hours.

It is further preferred that in step e) of the method of the present invention, the incubation of the mixture formed by steps a)-d) is carried out in an aqueous medium at a temperature being in the range from 30° C. to 60° C. for 6 to 8 hours. It is further preferred that in step e) of the method of the present invention, the incubation of the mixture formed by steps a)-d) is carried out in an aqueous medium at a temperature being in the range from 35° C. to 50° C. for 6 to 8 hours. It is further preferred that in step e) of the method of the present invention, the incubation of the mixture formed by steps a)-d) is carried out in an aqueous medium at a temperature being in the range from 35° C. to 45° C. for 6 to 8 hours. It is more preferred that in step e) of the method of the present invention, the incubation of the mixture formed by steps a)-d) is carried out in an aqueous medium at a temperature of about 40° C. for 6 to 8 hours.

This method of preparing a biocompatible hydrogel may comprise with step e) a step comprising thermo-induced gelation, which e.g. allows formation of specific form factors, e.g. by complete bubble-free filling of a suitably formed mould or by dropping a mixture of the two non-viscous solutions of the individual components into a lipophilic organic medium to form droplets of defined diameter. Formation of a covalent, non-degradable network of a biocompatible hydrogel can be induced by heating to mild temperatures as described above, which is compatible with maintaining enzymatic activity, when an enzyme is encapsulated therein. The method may comprise crosslinking via 1,3-dipolar cycloaddition, thermo-gelation under very mild reaction conditions (e.g. 40° C. in aqueous media) with no side reaction and no toxic reagent (e.g. glutaraldehyde). Optimization of pore sizes of the biocompatible hydrogel is possible by feasible adjustment of parameters and adjusting the degree of optional carboxymethylation. If an enzyme is added as described in step d), the one or more enzyme(s) will be immobilized in the produced biocompatible hydrogel, wherein the one or more enzyme(s) is/are then significantly longer stable and active than the free enzyme in solution at ambient or elevated temperature, e.g. body temperature, 37° C. Unstable sensitive enzymes, like alcohol oxidase, or glucose oxidase, have better life time performances under these conditions. This embodiment is also applicable to other sensitive enzymes. Such a produced biocompatible hydrogel can be stored dry without losing higher amounts of enzyme activity. Further, no or little leaching of enzyme can be achieved. Such hydrogels prepared according to this method of the present invention can be suspended in aqueous or organic solvents, while maintaining enzymatic activity (e.g. acetone). The viscosity of the individual components, as well as of the mixture can be easily adjusted.

Consequently, the stability of enzymes can be significantly improved by the encapsulation described herein, resulting in longer usable enzymes.

In one embodiment of the method of preparing a biocompatible hydrogel, the first polysaccharide and the second polysaccharide are independently from each other selected from the group consisting of pullulan, alginate, cellulose, hyaluronan, dextran, lichenin, lentinan and mixtures thereof. In one embodiment of the method of preparing a biocompatible hydrogel, the first polysaccharide and the second polysaccharide are independently from each other selected from the group consisting of pullulan, alginate, hyaluronan, dextran, lichenin, lentinan and mixtures thereof. In one embodiment of the method of preparing a biocompatible hydrogel, the first polysaccharide and the second polysaccharide are independently from each other selected from the group consisting of pullulan, alginate, hyaluronan, dextran, lentinan and mixtures thereof. In one embodiment of the method of preparing a biocompatible hydrogel, the first polysaccharide and the second polysaccharide are independently from each other selected from the group consisting of pullulan, alginate, hyaluronan, dextran and mixtures thereof. The polysaccharides pullulan, alginate, hyaluronan, dextran are defined herein above.

Cellulose is an organic compound with the formula (C₆H₁₀O₅)ₙ, a polysaccharide consisting of a linear chain of several hundred to many thousands of β(1→4)-linked D-glucose units. Cellulose is an important structural component of the primary cell wall of green plants, many forms of algae and the oomycetes. Some species of bacteria secrete it to form biofilms. In the context of the present invention, the respective monomeric repeating unit for cellulose has the structure of

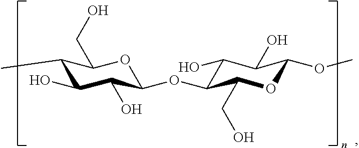

with n being the number of said monomeric repeating unit of cellulose, and with n being an integer from 10 to 10000.

Lichenin, also known as lichenan or moss starch, is a complex glucan occurring in certain species of lichens. It is chemically a mixed-linkage glycan, consisting of repeating glucose units linked by β-1,3- and β-1,4-glycosidic bonds. In the context of the present invention, the respective monomeric repeating unit for lichenin has the structure of

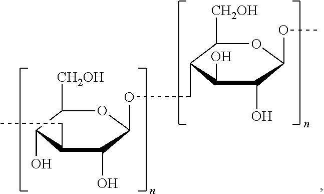

with n being the number of said monomeric repeating unit of lichenin, and with n being an integer from 10 to 10000.

Lentinan is a polysaccharide isolated from the fruit body of the shiitake mushroom. Chemically, lentinan is a β-1,3 beta-glucan with β-1,6 branching. In the context of the present

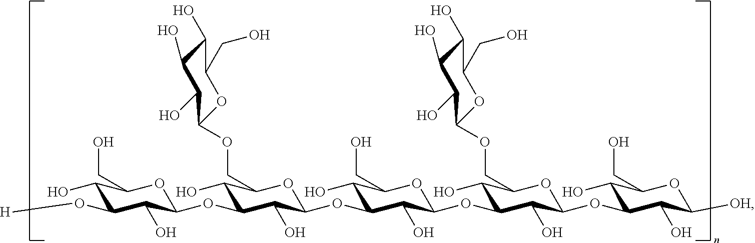

with n being the number of said monomeric repeating unit of lentinan, and with n being an integer from 10 to 10000.

In one embodiment of the method of preparing a biocompatible hydrogel, the first polysaccharide and the second polysaccharide are independently from each other selected from the group consisting of pullulan, alginate, cellulose, hyaluronan, dextran, lichenin and lentinan. In one embodiment of the method of preparing a biocompatible hydrogel, the first polysaccharide and the second polysaccharide are independently from each other selected from the group consisting of pullulan, alginate, hyaluronan, dextran, lichenin and lentinan. In one embodiment of the method of preparing a biocompatible hydrogel, the first polysaccharide and the second polysaccharide are independently from each other selected from the group consisting of pullulan, alginate, hyaluronan, dextran and lentinan. In a further embodiment of the method of preparing a biocompatible hydrogel, the first polysaccharide and the second polysaccharide are independently from each other selected from the group consisting of pullulan, alginate, hyaluronan and dextran. The respective monomeric repeating unit for each of these polysaccharides is as defined above herein.

In one preferred embodiment of the method of preparing a biocompatible hydrogel, the first polysaccharide and second polysaccharide are each pullulan.

In one embodiment of the method of preparing a biocompatible hydrogel, the first and/or second polysaccharide is dextran or pullulan and carboxymethylation of at least one OH-group of dextran or pullulan is carried out in step b). It is preferred for this embodiment that the carboxymethylation of at least one OH-group of dextran or pullulan is carried out in step b) at C₆ of dextran and/or pullulan. It is preferred for this embodiment that the carboxymethylation of at least one OH-group of dextran or pullulan is carried out in step b) at C₂ of dextran and/or pullulan. It is preferred for this embodiment that the carboxymethylation of at least one OH-group of dextran or pullulan is carried out in step b) at C₃ of dextran and/or pullulan. It is preferred for this embodiment that the carboxymethylation of at least one OH-group of dextran or pullulan is carried out in step b) at C₄ of dextran and/or pullulan.

In a further embodiment of the method of preparing a biocompatible hydrogel, in step c) the first polysaccharide is functionalized with 0.01-1.5 of A per monomeric repeating unit of the first polysaccharide. In a further preferred embodiment of the method of preparing a biocompatible hydrogel, in step c) the first polysaccharide is functionalized with 0.05-1.5 of A per monomeric repeating unit of the first polysaccharide. In a further preferred embodiment of the method of preparing a biocompatible hydrogel, in step c) the first polysaccharide is functionalized with 0.05-1.0 of A per monomeric repeating unit of the first polysaccharide.

For the method of preparing a biocompatible hydrogel according to the present invention, it is preferred that in step c) the second polysaccharide is functionalized with 0.01-1.5 of A′ per monomeric repeating unit of the second polysaccharide. In a further preferred embodiment of the method of preparing a biocompatible hydrogel according to the present invention, in step c) the second polysaccharide is functionalized with 0.05-1.5 of A′ per monomeric repeating unit of the second polysaccharide. In a further preferred embodiment of the method of preparing a biocompatible hydrogel according to the present invention, in step c) the second polysaccharide is functionalized with 0.05-1.0 of A′ per monomeric repeating unit of the second polysaccharide.

In a further embodiment of the method of preparing a biocompatible hydrogel, in step c) the first polysaccharide is functionalized with 0.01-1.5 of X per monomeric repeating unit of the first polysaccharide. In a further preferred embodiment of the method of preparing a biocompatible hydrogel according to the present invention, in step c) the first polysaccharide is functionalized with 0.05-1.5 of X per monomeric repeating unit of the first polysaccharide. In a further preferred embodiment of the method of preparing a biocompatible hydrogel according to the present invention, in step c) the first polysaccharide is functionalized with 0.05-1.0 of X per monomeric repeating unit of the first polysaccharide.

For the method of preparing a biocompatible hydrogel according to the present invention, it is preferred that in step c) the second polysaccharide is functionalized with 0.01-1.5 of Y per monomeric repeating unit of the second polysaccharide. In a further preferred embodiment of the method of preparing a biocompatible hydrogel according to the present invention, in step c) the second polysaccharide is functionalized with 0.05-1.5 of Y per monomeric repeating unit of the second polysaccharide. In a further preferred embodiment of the method of preparing a biocompatible hydrogel according to the present invention, in step c) the second polysaccharide is functionalized with 0.05-1.0 of Y per monomeric repeating unit of the second polysaccharide.

In one embodiment of the method of preparing a biocompatible hydrogel according to the present invention, d is 1.

It is further preferred for the method of preparing a biocompatible hydrogel according to the present invention, that step e) is a thermo-induced cycloaddition reaction between X and Y for forming a crosslinked polymer.

In one embodiment of the method of preparing a biocompatible hydrogel according to the present invention, the content of N₃ is 0.01-1.5 N₃ per monomeric repeating unit of the first polysaccharide. In one preferred embodiment of the method of preparing a biocompatible hydrogel according to the present invention, the content of N₃ is 0.01-1.0 N₃ per monomeric repeating unit of the first polysaccharide. In one further preferred embodiment of the method of preparing a biocompatible hydrogel according to the present invention, the content of N₃ is 0.1-1.0 N₃ per monomeric repeating unit of the first polysaccharide.

In one embodiment of the method of preparing a biocompatible hydrogel according to the present invention, the content of Q is 0.01-1.5 per monomeric repeating unit of the second polysaccharide. In one preferred embodiment of the method of preparing a biocompatible hydrogel according to the present invention, the content of Q is 0.01-1.0 per monomeric repeating unit of the second polysaccharide. In one preferred embodiment of the method of preparing a biocompatible hydrogel according to the present invention, the content of Q is 0.1-1.0 per monomeric repeating unit of the second polysaccharide.

In one preferred embodiment of the method of preparing a biocompatible hydrogel according to the present invention, in step c) -A-X is linked to at least one primary or secondary OH-group of the first polysaccharide, preferably via at least one of C₂, C₃, C₄ or C₆ of the monomeric repeating unit of the first polysaccharide, more preferably via C₆ of the monomeric repeating unit of the first polysaccharide.

In one preferred embodiment of the method of preparing a biocompatible hydrogel according to any one of the preceding claims, wherein in step c) -A′-Y is linked to at least one primary or secondary OH-group of the second polysaccharide, preferably via at least one of C₂, C₃, C₄ or C₆ of the monomeric repeating unit of the second polysaccharide, more preferably via C₆ of the monomeric repeating unit of the second polysaccharide.

It is further preferred for the method of preparing a biocompatible hydrogel according to the present invention, that the method is without the use of toxic reagents, preferably without the use of glutaraldehyde.

In one embodiment of the method of preparing a biocompatible hydrogel according to the present invention, the molecular weight of the unfunctionalized first polysaccharide is in the range from 5 to 2000 kDa. In one embodiment of the method of preparing a biocompatible hydrogel according to the present invention, the molecular weight of the functionalized first polysaccharide is in the range from 5 to 2500 kDa. In one preferred embodiment of the method of preparing a biocompatible hydrogel according to the present invention, the molecular weight of the functionalized first polysaccharide is in the range from 5 to 2000 kDa. In one more preferred embodiment of the method of preparing a biocompatible hydrogel according to the present invention, the molecular weight of the functionalized first polysaccharide is in the range from 5 to 1500 kDa. In one even more preferred embodiment of the method of preparing a biocompatible hydrogel according to the present invention, the molecular weight of the functionalized first polysaccharide is in the range from 10 to 1500 kDa.

In one embodiment of the method of preparing a biocompatible hydrogel according to the present invention, the molecular weight of the unfunctionalized second polysaccharide is in the range from 5 to 2000 kDa. In one embodiment of the method of preparing a biocompatible hydrogel according to the present invention, the molecular weight of the functionalized second polysaccharide is in the range from 5 to 2500 kDa. In one preferred embodiment of the method of preparing a biocompatible hydrogel according to the present invention, the molecular weight of the functionalized second polysaccharide is in the range from 5 to 2000 kDa. In one more preferred embodiment of the method of preparing a biocompatible hydrogel according to the present invention, the molecular weight of the functionalized second polysaccharide is in the range from 5 to 1500 kDa. In one even more preferred embodiment of the method of preparing a biocompatible hydrogel according to the present invention, the molecular weight of the functionalized second polysaccharide is in the range from 10 to 1500 kDa.

The present invention further provides the hydrogel obtainable by any method of preparing a biocompatible hydrogel as described above. It is preferred that said hydrogel comprises one or more encapsulated enzyme(s). It is further preferred that the hydrogel is a swellable or swollen hydrogel matrix.

In a further aspect, the present invention provides a biocompatible hydrogel comprising: a crosslinked polymer comprising the following structure:
a first polysaccharide and a second polysaccharide,
   preferably wherein the first polysaccharide and/or the second polysaccharide being independently selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

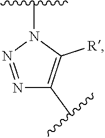

wherein R′ is selected from the group consisting of —CF₃, —C(O)—OMe, —C(O)—OEt, —C(O)—OH, —C(O)—NH₂ and —C(O)—NHMe, for non-covalent immobilization of one or more enzyme(s).

In a further aspect, the present invention provides a biocompatible hydrogel comprising: a crosslinked polymer comprising the following structure:
a first polysaccharide and a second polysaccharide,
   preferably wherein the first polysaccharide and/or the second polysaccharide being independently selected from the group consisting of pullulan, alginate, hyaluronan and dextran,
with n being the number of the monomeric repeating unit of the first and/or second polysaccharide, and
with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

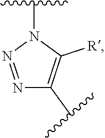

wherein R′ is selected from the group consisting of —CF₃, —C(O)—OMe, —C(O)—OEt, —C(O)—OH, —C(O)—NH₂ and —C(O)—NHMe,
   for non-covalent immobilization of one or more enzyme(s).

In a further aspect, the present invention provides a biocompatible hydrogel comprising:
a crosslinked polymer comprising the following structure:
   a first polysaccharide and a second polysaccharide,
wherein the first polysaccharide and/or the second polysaccharide being independently selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

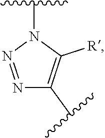

wherein R′ is selected from the group consisting of —CF₃, —C(O)—OMe, —C(O)—OEt, —C(O)—OH, —C(O)—NH₂ and —C(O)—NHMe,
   for non-covalent immobilization of one or more enzyme(s).

In a further aspect, the present invention provides a biocompatible hydrogel comprising:
a crosslinked polymer comprising the following structure:
   a first polysaccharide and a second polysaccharide,
wherein the first polysaccharide and/or the second polysaccharide being independently selected from the group consisting of pullulan, alginate, hyaluronan, dextran and mixtures thereof,
with n being the number of the monomeric repeating unit of the first and/or second polysaccharide, and
with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

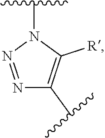

wherein R′ is selected from the group consisting of —CF₃, —C(O)—OMe, —C(O)—OEt, —C(O)—OH, —C(O)—NH₂ and —C(O)—NHMe,
for non-covalent immobilization of one or more enzyme(s).

The above given definitions for the method of preparing a biocompatible hydrogel also apply to the biocompatible hydrogel, if the same terms are used. As used herein for the biocompatible hydrogel according to the invention, the parameters r, s and t—defined herein as r being an integer from 2 to 20, s being an integer from 1 to 15 and t being an integer from 1 to 15—apply for both, Z₁ and Z₂.

In one preferred embodiment, the biocompatible hydrogel comprises: a crosslinked polymer comprising the following structure:
a first polysaccharide and a second polysaccharide, wherein the first polysaccharide and/or the second polysaccharide being independently selected from the group consisting of pullulan, alginate, hyaluronan and dextran,
   with n being the number of the monomeric repeating unit of the first and/or second polysaccharide, and
   with n being an integer from 10 to 10000,
one or more linker unit(s), which link the first polysaccharide with the second polysaccharide, wherein the structure of the one or more linker unit(s) is

-A-Z₁—B—Z₂-A′-,

wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is

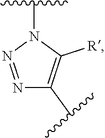

wherein R′ is selected from the group consisting of —CF₃, —C(O)—OMe, —C(O)—OEt, —C(O)—OH, —C(O)—NH₂ and —C(O)—NHMe,
for non-covalent immobilization of one or more enzyme(s).

In one embodiment of the biocompatible hydrogel according to the present invention, the hydrogel is a swellable or swollen hydrogel matrix.

In one further embodiment of the biocompatible hydrogel according to the present invention, the non-covalent immobilization of the one or more enzyme(s) comprises encapsulation of the one or more enzyme(s) and non-covalent binding of the one or more enzyme(s) in the hydrogel. As used herein and in the context of the present invention, the term “non-covalent” means an interaction, which differs from a covalent bond in that it does not involve the sharing of a bond, but rather involves more dispersed variations of electromagnetic interactions between molecules or within a molecule like dipol-dipol or charge-charge interactions. The chemical energy released in the formation of non-covalent interactions is typically in the order of 1-5 kcal/mol. Non-covalent interactions can be classified into different categories, such as electrostatic, π-effects, van der Waals forces, and hydrophobic effects. Non-covalent interactions are critical in maintaining the three-dimensional structure of large molecules, such as proteins and nucleic acids. In addition, they are also involved in many biological processes in which large molecules bind specifically, but transiently, to one another.

In one embodiment of the biocompatible hydrogel according to the present invention, the first and/or second polysaccharide are independently from each other selected from the group consisting of pullulan, alginate, cellulose, hyaluronan, dextran, lichenin, lentinan and mixtures thereof.

In one further embodiment of the biocompatible hydrogel according to the present invention, the first and/or second polysaccharide is/are independently from each other selected from the group consisting of pullulan, alginate, hyaluronan, dextran, lichenin, lentinan and mixtures thereof. In one further embodiment of the biocompatible hydrogel according to the present invention, the first and/or second polysaccharide is/are independently from each other selected from the group consisting of pullulan, alginate, hyaluronan, dextran, lentinan and mixtures thereof. In one further embodiment of the biocompatible hydrogel according to the present invention, the first and/or second polysaccharide is/are independently from each other selected from the group consisting of pullulan, alginate, hyaluronan, dextran and mixtures thereof.

In one embodiment of the biocompatible hydrogel according to the present invention, the first and/or second polysaccharide is/are pullulan.

In one further preferred embodiment of the biocompatible hydrogel according to the present invention, -A-Z₁— has the formula —CH₂—CO—NH—(CH₂—CH₂—O)ₙ—CH₂—CH₂—, wherein n is preferably from 1 to 5.

In one embodiment of the biocompatible hydrogel according to the present invention, the first and/or the second polysaccharide has/have a concentration of 5-120 mg/ml, preferably 10-80 mg/ml, more preferably 20-60 mg/ml, with respect to the total hydrogel.

In one further embodiment of the biocompatible hydrogel according to the present invention, the one or more enzyme(s) for non-covalent immobilization may be selected from the group consisting of lipases and oxidases, preferably glucose oxidase, lactate oxidase, uricase, glutamate oxidase, cortisol oxidase, xanthine oxidase, cholesterol oxidase, sarcosine oxidase, and alcohol oxidase.

The present invention further provides a composition comprising the biocompatible hydrogel according to the present invention and as defined herein.

The present invention further provides a method for encapsulating one or more enzyme(s) in a biocompatible hydrogel according to the present invention and as defined herein. Said method comprises the contacting of said biocompatible hydrogel with said one or more enzyme(s) and afterwards the incubation thereof in an aqueous medium at a temperature being in the range from 25° C. to 70° C., preferably at about 40° C. for at least one hour, preferably for 1 to 10 hours. Said method preferably comprises the contacting of said biocompatible hydrogel with said one or more enzyme(s) and afterwards the incubation thereof in an aqueous medium at a temperature being in the range from 25° C. to 70° C. for 1-15 hours. Said method more preferably comprises the contacting of said biocompatible hydrogel with said one or more enzyme(s) and afterwards the incubation thereof in an aqueous medium at a temperature being in the range from 25° C. to 70° C. for 1-10 hours.

In a further aspect, the present invention provides the use of a hydrogel according to the present invention and as defined herein,
a) for non-covalent immobilization of one or more enzyme(s) in the hydrogel, or
b) in a biosensor. As used herein, the term “biosensor” means, unlike biotic sensors or biotests, a self-contained integrated system that provides specific quantitative or semi-quantitative analytical information, consisting of biological recognition element (biochemical receptor or enzyme) and transducers (e.g. an electrode) in direct spatial contact.

The present invention also provides a kit comprising the composition or the hydrogel according to the present invention and as defined herein.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein, the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES

### Materials and Methods

### Synthesis of Oxanorbornadiene Linker

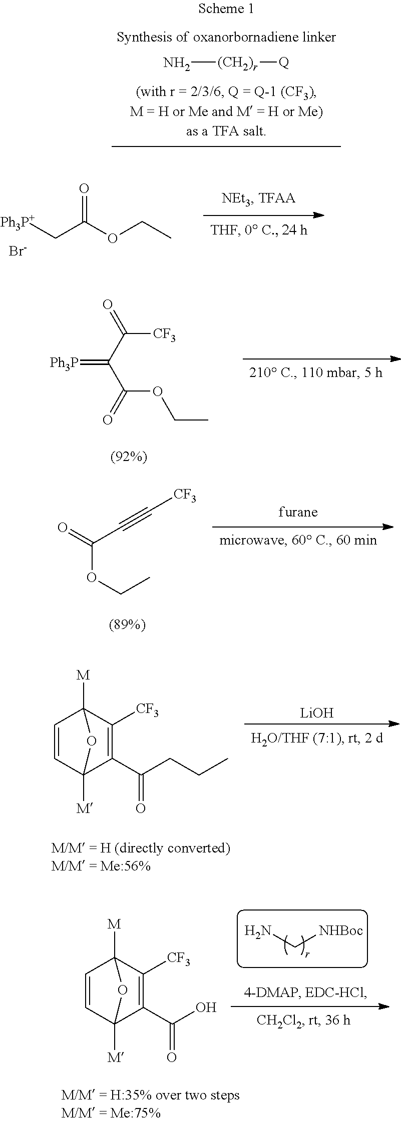

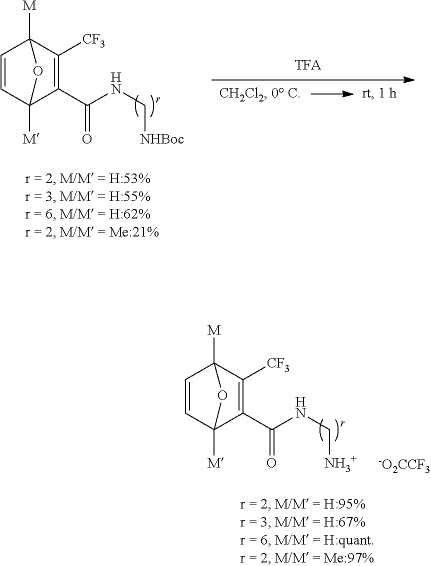

### Ethyl 4,4,4-trifluoro-3-oxo-2-(triphenyl-λ⁵-phosphaneylidene)-butanoate (Compound 1)

At 0° C., (2-ethoxy-2-oxoethyl)triphenylphosphonium bromide (100 g, 0.233 mol) was dissolved in dry THF (400 mL) under argon atmosphere. Triethylamine (65 mL, 0.47 mol) was slowly added to the reaction mixture over 20 min. Trifluoroacetic anhydride (36 mL, 0.26 mol) was then added dropwise. The reaction was stirred for 24 h while slowly warming to rt. The precipitate was filtered off and washed with cold THF. The solvent of the filtrate was removed on the rotary evaporator and H₂O/THF (3:1, 400 mL) was added to the residue. The precipitated pale yellow solid was then filtered off, dried in vacuo at 40° C., and recrystallized several times from methanol/H₂O. The product (90.9 g, 0.205 mol, 88%) was obtained as a crystalline solid.

HR-MS: m/z calculated for C₂₄H₂₀F₃O₃PNa⁺[M+Na]⁺: 467.1000. found (ESI-MS⁺): 467.0997.

¹H-NMR (600 MHz, DMSO): δ=7.73-7.59 (m, 15H, CH_{(Ph)}), 3.63 (q, 2H, CH₂CH₃, J=7.1 Hz), 0.69 (t, 3H, CH₂CH₃, J=7.1 Hz) ppm.

¹³C{¹H}-NMR (151 MHz, DMSO) δ=172.6 (qd, J=55.2, 6.4 Hz, COCF₃), 164.5 (d, J=12.1 Hz, CO₂Et), 132.8 (d, J=10.1 Hz, CH_{(Ph)}), 132.7 (d, J=2.9 Hz, CH_{(Ph)}), 129.2 (d, J=12.6 Hz, CH_{(Ph)}), 123.4 (d, J=93.2 Hz, C_{q}(Pₕ)), 115.8 (qd, J=14.9, 483.8 Hz, CF₃), 69.3 (d, J=109.7 Hz, C═P), 59.2 (s, CH₂CH₃), 13.2 (s, CH₂CH₃) ppm.

### Ethyl 4,4,4-trifluorobut-2-ynoate (Compound 2)

Ethyl 4,4,4-trifluoro-3-oxo-2-(triphenyl-λ⁵-phosphaneylidene)butanoate (10 g, 23 mmol) distilled over a pyrolysis apparatus for 4-5 h at 220° C. and 100-160 mbar. The product (3.30 g, 19.9 mmol, 89%) was obtained as a clear colorless liquid.

¹H-NMR (400 MHz, CDCl₃): δ=4.32 (q, J=7.1 Hz, 2H, CH₂), 1.34 (t, J=7.2 Hz, 3H, CH₃) ppm.

¹³C{¹H}-NMR (101 MHz, CDCl₃) δ=151.0 (q, J=1.7 Hz, C═O), 113.6 (q, J=260.1 Hz, CF₃), 75.8 (q, J=6.5 Hz, CCF₃), 70.1 (q, J=54.8 Hz, CC═O), 63.7 (s, CH₂CH₃), 14.0 (s, CH₂CH₃) ppm.

### Ethyl 1,4-dimethyl-3-(trifluoromethyl)-7-oxabicyclo[2.2.1]hepta-2,5-diene-2-carboxylate (Compound 3)

Ethyl 4,4,4-trifluorobut-2-ynoate (3.30 g, 19.9 mmol) was mixed with 2,5-dimethylfuran (3.15 mL, 29.8 mmol) and heated in a microwave reactor to 60° C. for 60 min. The reaction mixture was concentrated by coevaporation with toluene (3×). The product was obtained as a brown oily residue (2.91 g, 0.11 mol, 56%).

HR-MS: m/z calculated for C₁₂H₁₃F₃O₃Na⁺: 285.0714 [M+Na]⁺. found (ESI-MS⁺): 285.0718.

¹H-NMR (400 MHz, DMSO): δ=7.15 (d, J=5.0 Hz, 1H, HC═CH), 7.13 (d, J=5.1 Hz, 1H, HC═CH), 4.34-4.19 (m, 2H, CH₂CH₃), 1.72 (s, 3H, CH₃), 1.64 (s, 3H, CH₃), 1.23 (t, J=7.1 Hz, 3H, CH₂CH₃) ppm.

¹³C{¹H}-NMR (101 MHz, DMSO) δ=163.1 (q, J=1.5 Hz, C═O), 155.9 (q, J=4.9 Hz, CCF₃), 147.5 (q, J=34.2 Hz, CC═O), 147.5 (d, J=0.7 Hz, CCH₃), 146.9 (s, CCH₃), 122.0 (q, J=269.5 Hz, CF₃), 92.3 (d, J=0.5 Hz, HC═CH), 91.0 (q, J=1.4 Hz, HC═CH), 61.6 (s, CH₂CH₃), 14.7 (d, J=1.0 Hz, CH₂CH₃), 14.4 (s, CH₃), 13.7 (s, CH₃).

### 3-(Trifluoromethyl)-7-oxabicyclo[2.2.1]hepta-2,5-diene-2-carboxylic acid (Compound 4)

In a round bottom flask, Ethyl 4,4,4-trifluoro-3-oxo-2-(triphenyl-λ⁵-phosphaneylidene)butanoate (38.8 g, 87.4 mmol) was distilled over a pyrolysis apparatus for 4-5 h at 210° C. and 100-160 mbar. Furan (38.0 mL, 52.4 mmol) was then added to the colorless distillate (ethyl 4,4,4-trifluorobut-2-ynoate) and reacted in a microwave reactor at 60° C. for 60 min. The reaction mixture was concentrated by co-evaporation with toluene (3×) under reduced pressure. The ester could be obtained as an oily residue and was taken up without further purification in H₂O/THF (7:1, 100 mL), mixed with 1 M aq. LiOH solution (100 mL, 100 mM) and stirred at rt for three days. The aqueous phase was then adjusted to pH 1-2 with 2 M HCl and extracted with diethyl ether (3×). The combined organic phases were dried over Na₂SO₄ and the solvent was removed under reduced pressure. The oily residue was dissolved in dichloromethane and petroleum ether was added. The precipitated solid was filtered off and washed with petroleum ether. This afforded the product (6.16 g, 29.9 mmol, 35% over three steps) as a crystalline solid.

HR-MS: m/z calculated for C₃H₄F₃O₃⁻ [M−H]⁻: 205.0113. found (ESI-MS⁻): 205.0108.

¹H-NMR (600 MHz, DMSO): δ=13.75 (bs, 1H, COOH), 7.38 (dd, J=5.3 Hz, 2.0 Hz, 1H, HC═CH), 7.34 (dd, J=5.3 Hz, 2.0 Hz, 1H, HC═CH), 5.86 (t, J=1.7 Hz, 1H, HC—O), 5.73 (d, J=1.3 Hz, 1H, HC—O) ppm.

¹³C{¹H}-NMR (151 MHz, DMSO) δ=163.2 (q, J=1.5 Hz, COOH), 154.3 (q, J=4.9 Hz, CCF₃), 147.4 (q, J=36.3 Hz, CC═O), 143.9 (s, HC—O), 143.1 (s, HC—O), 122.0 (q, J=268.8 Hz, CF₃), 84.8 (s, HC═CH), 83.0 (q, J=2.5 Hz, HC═CH) ppm.

### 1,4-dimethyl-3-(trifluoromethyl)-7-oxabicyclo[2.2.1]hepta-2,5-diene-2-carboxylic acid (Compound 5)

Ethyl 1,4-dimethyl-3-(trifluoromethyl)-7-oxabicyclo[2.2.1]hepta-2,5-diene-2-carboxylate (191 mg, 0.73 mmol) was taken up in H₂O/THF (7:1, 2 mL), mixed with 1 M aq. LiOH solution (2 mL, 2 mM) and stirred at rt for 1.5 days. The aqueous phase was then brought to pH 1-2 with 2 M HCl and extracted with diethyl ether (3×). The combined organic phases were dried over Na₂SO₄ and the solvent was removed under reduced pressure. The product (127 mg, 0.54 mmol, 75%) was obtained as a brown oil.

HR-MS: m/z calculated for C₁₀H₃F₃O₃⁻ [M−H]⁻: 233.0426. found (ESI-MS⁻): 233.0422.

¹H-NMR (400 MHz, DMSO): δ=13.93 (bs, 1H, COOH), 7.12 (d, J=5.1 Hz, 1H, HC═CH), 7.10 (d, J=5.1 Hz, 1H, HC═CH), 1.71 (s, 3H, CH₃), 1.63 (s, 3H, CH₃) ppm.

¹³C{¹H}-NMR (101 MHz, DMSO) δ=164.9 (q, J=1.4 Hz, COOH), 157.1 (q, J=4.9 Hz, CCF₃), 147.4 (s, HC—O), 146.8 (s, HC—O), 145.1 (q, J=34.0 Hz, CC═O), 122.3 (q, J=269.2 Hz, CF₃), 92.2 (d, J=0.6 Hz, HC═CH), 90.7 (q, J=1.4 Hz, HC═CH), 14.82 (s, CH₃), 14.5 (s, CH₃) ppm.

### Tert-butyl (2-(3-(trifluoromethyl)-7-oxabicyclo[2.2.1]hepta-2,5-diene-2-carboxamido)ethyl)carbamate (Compound 6)

3-(Trifluoromethyl)-7-oxabicyclo[2.2.1]hepta-2,5-diene-2-carboxylic acid (1.18 g, 5.73 mmol) was dissolved in dry dichloromethane (16 mL) under argon atmosphere. After stepwise addition of 4-DMAP (1.4 g, 11 mmol), EDC-HCl (1.6 g, 8.6 mmol), and Boc-ethylenediamine (1.1 mL, 6.9 mmol), the mixture was stirred for 22 h at rt. The reaction mixture was washed with sat. aq. NaCl solution, the organic phase was dried over Na₂SO₄, and the solvent was removed under reduced pressure. After the crude product was purified by column chromatography (eluent: PE/EtOAc 1:1), the product was obtained as a white solid (1.01 g, 2.91 mmol, 51%).

R_{f}: (PE/EtOAc: 1/1): 0.45.

HR-MS: m/z calculated for C₁₅H₁₉F₃N₂O₄Na⁺: 371.1195 [M+Na]⁺. found (ESI-MS⁺): 371.1189.

¹H-NMR (500 MHz, DMSO): δ=8.46 (t, J=5.5 Hz, 1H, NH—C═O), 7.33 (dd, J=5.3, 2.0 Hz, 1H, HC═CH), 7.28 (dd, J=5.3, 2 Hz, 1H, HC═CH), 6.83 (t, J=5.7 Hz, 1H, NH-Boc), 5.79 (t, 1H, HC—O), 5.67 (d, J=1.0 Hz, 1H, HC—O), 3.18-3.15 (m, 2H, CH₂—NH), 3.04-3.00 (m, 2H, CH₂—Boc), 1.38 (s, 9H, Boc) ppm.

¹³C{¹H}-NMR (126 MHz, DMSO): δ=161.9 (q, J=1.2 Hz, CONH), 155.8 (q, J=5.1 Hz, CCF₃), 155.6 (s, NC═O), 143.6 (d, J=0.7 Hz, HC═CH), 142.7 (s, HC═CH), 141.7 (q, J=35.7 Hz, CC═O), 122.4 (q, J=268.2 Hz, CF₃), 85.1 (s, HC—O), 82.4 (q, J=2.1 Hz, HC—O), 77.7 (s, C_{q(Boc)}), 39.1 (s, NHCH₂), 38.9 (s, NHCH₂), 28.2 (s, CH_{3(Boc)}).

### Tert-butyl (2-(1,4-dimethyl-3-(trifluoromethyl)-7-oxabicyclo[2.2.1]hepta-2,5-diene-2-carboxamido)ethyl)carbamate (Compound 7)

1,4-dimethyl-3-(trifluoromethyl)-7-oxabicyclo[2.2.1]hepta-2,5-diene-2-carboxylic acid (0.68 g, 2.90 mmol) was dissolved in dry dichloromethane (12 mL) under Ar atmosphere. After stepwise addition of 4-DMAP (0.71 g, 5.8 mmol), EDC-HCl (0.83 g, 4.3 mmol), and Boc-ethylenediamine (0.55 mL, 3.5 mmol), the mixture was stirred for 20 h at rt. The reaction mixture was washed with sat. aq. NaCl solution, the organic phase was dried over Na₂SO₄, and the solvent was removed under reduced pressure. After the crude product was purified by column chromatography (eluent: PE/EtOAc 3:1; 1:1), the product was obtained as oil (0.23 g, 0.60 mmol, 21%).

R_{f}: (PE/EtOAc: 1/1): 0.63.

HR-MS: m/z calculated for C₁₇H₂₃F₃N₂O₄Na⁺: 399.1508 [M+Na]⁺. found (ESI-MS⁺): 399.1502.

¹H-NMR (400 MHz, DMSO): δ=8.56 (t, J=5.4 Hz, 1H, NH—CO), 7.06 (d, J=5.0 Hz, 1H, HC═CH), 7.03 (d, J=5.0 Hz, 1H, HC═CH), 6.77 (t, J=5.5 Hz, 1H, NH-Boc), 3.27-3.08 (m, 2H, CH₂—NH), 3.05-2.93 (m, 2H, CH₂—NHBoc), 1.70 (s, 3H, CH₃), 1.55 (s, 3H, CH₃), 1.37 (s, 9H, Boc) ppm.

¹³C{¹H}-NMR (101 MHz, DMSO): δ=163.0 (s, CONH), 155.7 (q, J=5.0 Hz, CCF₃), 155.5 (s, NC═O), 147.3 (s, HC═CH), 146.2 (s, HC═CH), 140.9 (q, J=33.2 Hz, CC═O), 122.6 (q, J=268.9 Hz, CF₃), 92.3 (s, HC—O), 90.4 (q, J=1.3 Hz, HC—O), 77.7 (s, C_{q(Boc)}), 39.2 (s, NHCH₂), 38.5 (s, NHCH₂), 28.2 (s, CH_{3(Boc)}), 14.9 (s, CH₃), 14.1 (s, CH₃) ppm.

### Tert-butyl (3-(3-(trifluoromethyl)-7-oxabicyclo[2.2.1]hepta-2,5-diene-2-carboxamido)propyl)-λ²-azanecarboxylate (Compound 8)

3-(Trifluoromethyl)-7-oxabicyclo[2.2.1]hepta-2,5-diene-2-carboxylic acid (512 mg, 2.49 mmol) was dissolved in dry dichloromethane (6 mL) under Ar atmosphere. After stepwise addition of 4-DMAP (607 mg, 4.97 mmol), EDC-HCl (715 mg, 3.73 mmol), and N-Boc-1,3 diaminopropane (0.52 mL, 3.0 mmol), the mixture was stirred for 22 h at rt. The reaction mixture was washed with sat. aq. NaCl solution, the organic phase was dried over Na₂SO₄, and the solvent was removed under reduced pressure. After the crude product was purified by column chromatography (eluent: PE/EtOAc 1:1), the product was obtained as oil (528 mg, 1.46 mmol, 55%).

R_{f}: (PE/EtOAc: 3/2): 0.29.

HR-MS: m/z calculated for C₁₆H₂₁F₃N₂O₄Na⁺: 385.1351 [M+Na]⁺. found (ESI-MS⁺): 385.1360.

¹H-NMR (400 MHz, DMSO): δ=8.43 (t, J=5.6 Hz, 1H, C═ONH), 7.32 (dd, J=5.3, 2.0 Hz, 1H, HC═CH), 7.28 (dd, J=5.3, 2.0 Hz, 1H, HC═CH), 6.77 (t, J=5.4 Hz, 1H, NHBoc), 5.79 (t, J=1.5 Hz, 1H, HC—O), 5.65 (s, 1H, HC—O), 3.17-3.14 (m, 1H, CONCH₂), 3.13-3.07 (m, 1H, CONCH₂), 2.95-2.91 (m, 2H, CH₂NHBoc), 1.57-1.52 (m, 2H, CCH₂C), 1.37 (s, 9H, Boc).

¹³C{¹H}-NMR (101 MHz, DMSO) δ=161.8 (s, CONH), 155.7 (q, J=5.0 Hz, CCF₃), 155.6 (s, NC═O), 143.5 (s, HC═CH), 142.6 (s, HC═CH), 141.2 (q, J=35.6 Hz, CC═O), 122.5 (q, J=268.1 Hz, CF₃), 85.2 (s, HC—O), 82.4 (d, J=2.0 Hz, HC—O), 77.5 (s, C_{q(Boc)}), 37.4 (s, NHCH₂), 36.5 (s, NHCH₂), 29.2 (s, CCH₂C), 28.2 (s, CH_{3(Boc)}).

### Tert-butyl (6-(3-(trifluoromethyl)-7-oxabicyclo[2.2.1]hepta-2,5-diene-2-carboxamido)hexyl)-λ²-azanecarboxylate (Compound 9)

3-(Trifluoromethyl)-7-oxabicyclo[2.2.1]hepta-2,5-diene-2-carboxylic acid (0.50 g, 2.4 mmol) was dissolved in dry dichloromethane (10 mL) under Ar atmosphere. After stepwise addition of 4-DMAP (597 mg, 4.88 mmol), EDC-HCl (702 mg, 3.66 mmol), and N-Boc-1,6-diaminohexane (657 μL, 2.93 mmol), the mixture was stirred for 22 h at rt. The reaction mixture was washed with sat. aq. NaCl solution, the organic phase was dried over Na₂SO₄, and the solvent was removed under reduced pressure. After the crude product was purified by column chromatography (eluent: PE/EtOAc 7:1, 3:1, 1:1), the product was obtained as oil (610 mg, 1.51 mmol, 62% yield).

R_{f}: (PE/EtOAc: 3:2): 0.34.

HR-MS: m/z calculated for C₁₉H₂₇F₃N₂O₄Na⁺: 427.1821 [M+Na]⁺. found (ESI-MS⁺): 427.1830.

¹H-NMR (600 MHz, DMSO): δ=8.47 (t, J=5.6 Hz, CONH), 7.33 (dd, J=5.3, 2.0 Hz, 1H, HC═CH), 7.28 (dd, J=5.2, 1.9 Hz, 1H, HC═CH), 6.75 (t, J=5.3 Hz, 1H, NHBoc), 5.79 (t, J=1.5 Hz, 1H, HC—O), 5.65 (s, 1H, HC—O), 3.22-3.16 (in, 1H, CONCH₂), 3.10-3.04 (m, 1H, CONCH₂), 2.89 (q, J=6.6 Hz, 2H, CH₂NHBoc), 1.45-1.33 (m, 13H, CH_{3(Boc)}, CCH₂C), 1.28-1.22 (m, 4H, CCH₂C) ppm.

¹³C{¹H}-NMR (151 MHz, DMSO) δ=161.7 (s, CONH), 155.7 (q, J=5.1 Hz, CCF₃), 155.5 (s, NC═O) 143.5 (s, HC═CH), 142.5 (s, HC═CH), 140.9 (q, J=35.6 Hz, CC═O), 122.5 (q, J=268.1 Hz, CF₃), 85.2 (s, HC—O), 82.4 (d, J=2.0 Hz, HC—O), 77.2 (s, C_{q}Boc), 40.0 (s, NHCH₂), 38.5 (s, NHCH₂), 29.4 (s, CCH₂C), 28.7 (s, CCH₂C), 28.2 (s, CH_{3(Boc)}), 25.9 (s, CCH₂C).

### 2-(3-(Trifluoromethyl)-7-oxabicyclo[2.2.1]hepta-2,5-diene-2-carboxamido)ethan-1-ammonium trifluoroacetate (Compound 10)

Tert-butyl(2-(3-(trifluoromethyl)-7-oxabicyclo[2.2.1]hepta-2,5-diene-2-carboxamido)ethyl) carbamate (1.00 g, 2.87 mmol) was dissolved in dichloromethane (10 mL). At 0° C., TFA (8.8 mL, 0.12 mol) was added dropwise. After stirring for 30 min at the same temperature, no starting material could be observed by TLC and the reaction mixture was concentrated by aceotropic distillation with toluene (3×). Subsequently, the brown residue was treated with ethyl acetate and crystallized overnight at 4° C. The product was obtained after filtration as a crystalline solid (0.98 g, 2.7 mmol, 95%).

HR-MS: m/z calculated for C₁₀H₁₁F₃N₂O₂Na⁺: 271.0670 [M+H]⁺. found (ESI-MS⁺): 271.0681.

¹H-NMR (400 MHz, DMSO): δ=8.64 (t, J=5.6 Hz, 1H, NH-Boc), 7.94 (s, 3H, NH₃⁺), 7.34 (dd, J=5.2 Hz, 1.9 Hz, 1H, HC═CH), 7.30 (dd, J=5.2 Hz, 1.9 Hz, 1H, HC═CH), 5.81 (t, J=1.4 Hz, 1H, HC—O), 5.73 (d, J=1.2 Hz, 1H, HC—O), 3.48-3.32 (m, 2H, CH₂—NH), 2.91 (t, J=6.4 Hz, 2H, CH₂NH₃⁺) ppm.

¹³C{¹H}-NMR (101 MHz, CDCl₃) δ=162.3 (q, J=1.4 Hz, CONH), 158.3 (q, J=31.2 Hz, O₂CCF₃), 155.7 (q, J=5.2 Hz, CCF₃), 143.7 (d, J=0.8 Hz, HC═CH), 142.8 (q, J=35.9 Hz, CC═O), 142.8 (s, HC═CH), 122.4 (q, J=268.3 Hz, CCF₃), 85.2 (s, CH—O), 82.6 (q, J=2.2 Hz, CH—O), 38.1 (s, CH₂NHCO), 36.5 (s, CH₂NH₃⁺) ppm.

### 2-(1,4-dimethyl-3-(trifluoromethyl)-7-oxabicyclo[2.2.1]hepta-2,5-diene-2-carboxamido)ethan-1-ammonium trifluoroacetate (Compound 11)

Tert-butyl (2-(1,4-dimethyl-3-(trifluoromethyl)-7-oxabicyclo[2.2.1]hepta-2,5-diene-2-carboxamido)ethyl)carbamate (88.6 mg, 0.235 mmol) was dissolved in dichloromethane (2 mL). At 0° C., TFA (0.72 mL, 9.4 mmol) was added dropwise. After stirring for 30 min at the same temperature, no starting material could be observed by TLC and the reaction mixture was concentrated by aceotropic distillation with toluene (3×). The product was obtained as oil (88.9 mg, 0.228 mmol, 97%).

HR-MS: m/z calculated for C₁₂H₁₆F₃N₂O₂Na⁺: 277.1164 [M+H]⁺. found (ESI-MS⁺): 277.1164.

¹H-NMR (400 MHz, DMSO): δ=8.74 (t, J=5.6 Hz, 1H, NH-Boc), 7.92 (s, 3H, NH₃⁺), 7.08 (d, J=5.0 Hz, 1H, HC═CH), 7.05 (d, J=5.1 Hz, 1H, HC═CH), 3.47-3.32 (m, 2H, CH₂), 2.86 (q, J=6.1 Hz, 2H, CH₂), 1.70 (s, 3H, CH₃), 1.57 (s, 3H, CH₃) ppm.

¹³C{¹H}-NMR (126 MHz, DMSO) δ=163.4 (q, J=1.2 Hz, CONH), 158.4 (q, J=5.0 Hz, CCF₃), 158.1 (q, J=32.4 Hz, O₂CCF₃), 147.3 (s, HC═CH), 146.3 (s, HC═CH), 141.8 (q, J=33.4 Hz, CC═O), 122.5 (q, J=269.0 Hz, CCF₃), 92.3 (s, CH—O), 90.5 (q, J=1.3 Hz, CH—O), 37.9 (s, CH₂NHCO), 36.2 (s, CH₂NH₃⁺), 14.9 (s, CH₃), 14.1 (s, CH₃) ppm.

### 3-(3-(Trifluoromethyl)-7-oxabicyclo[2.2.1]hepta-2,5-diene-2-carboxamido)propan-1-ammonium trifluoroacetate (Compound 12)

Tert-butyl (3-(3-(trifluoromethyl)-7-oxabicyclo[2.2.1]hepta-2,5-diene-2-carboxamido)propyl)-λ²-azanecarboxylate (100 mg, 0.28 mmol) was dissolved in dichloromethane (2 mL). At 0° C., TFA (0.85 mL, 0.011 mol) was added dropwise. After stirring for 30 min at the same temperature, no starting material was observed by TLC and the reaction mixture was concentrated by aceotropic distillation with toluene (3×). The residue was then treated with ethyl acetate and diethyl ether and decanted. The product was obtained as an oil (66 mg, 0.18 mmol, 67%).

HR-MS: m/z calculated for C₁₁H₁₄F₃N₂O₂⁺: 263, 1007 [M+H]⁺. found (ESI-MS⁺): 263, 100.

¹H-NMR (400 MHz, DMSO): δ=8.65 (t, J=5.8 Hz, 1H, NH_{Amid}), 7.86 (s, 3H, NH₃⁺), 7.33 (dd, J=5.3, 2.0 Hz, 1H, HC═CH), 7.29 (dd, J=5.3, 1.9 Hz, 1H, HC═CH), 5.81 (t, J=1.6 Hz, 1H, HC—O), 5.68 (d, J=1.3 Hz, 1H, HC—O), 3.27 (m, 1H, CH₂—NHCO), 3.17 (m, 1H, CH₂—NHCO), 2.80 (q, J=6.9 Hz, 2H, CH₂NH₃⁺), 1.73 (quin, J=7.2 Hz, 2H, CH₂CH₂CH₂) ppm.

¹³C{¹H}-NMR (101 MHz, CDCl₃) δ=162.2 (d, J=1.3 Hz, CONH), 158.4 (q, J=31.9 Hz, O₂CCF₃), 155.6 (q, J=5.1 Hz, CCF₃), 143.6 (d, J=0.7 Hz, HC═CH), 142.7 (s, HC═CH), 141.7 (q, J=35.7 Hz, CC═O), 122.5 (q, J=268.2 Hz, CCF₃), 117.0 (q, J=298.3 Hz, O₂CCF₃), 85.2 (s, CH—O), 82.5 (q, J=2.1 Hz, CH—O), 36.7 (s, CH₂NHCO), 35.9 (s, CH₂NH₃⁺), 27.1 (s, CH₂CH₂CH₂) ppm.

### 6-(3-(Trifluoromethyl)-7-oxabicyclo[2.2.1]hepta-2,5-diene-2-carboxamido)hexan-1-ammonium trifluoroacetate (Compound 13)

Tert-butyl (6-(3-(trifluoromethyl)-7-oxabicyclo[2.2.1]hepta-2,5-diene-2-carboxamido)hexyl)-λ²-azanecarboxylate (205 mg, 0.507 mmol) was dissolved in dichloromethane (4 mL). At 0° C., TFA (1.55 mL, 0.020 mol) was added dropwise. After stirring for 30 min at the same temperature, no starting material was observed by TLC and the reaction mixture was concentrated by aceotropic distillation with toluene (3×). Subsequently, the residue was treated with diethyl ether and petroleum ether and decanted. The product was obtained as an oil (215 mg, 0.051 mmol, quant.).

HR-MS: m/z calculated for C₁₄H₁₉F₃N₂O₂⁺: 305.1477 [M+H]⁺. found (ESI-MS⁺): 305.1479.

¹H-NMR (500 MHz, DMSO): δ=8.51 (t, J=5.7 Hz, 1H, NH_{Amid}), 7.72 (s, 3H, NH₃⁺), 7.32 (dd, J=5.3, 2.0 Hz, 1H, HC═CH), 7.27 (dd, J=5.3, 1.9 Hz, 1H, HC═CH), 5.79 (t, J=1.6 Hz, HC—O), 5.64 (d, J=1.3 Hz, HC—O), 3.25-3.18 (m, 1H, CH₂—NHCO), 3.10-3.03 (m, 1H, CH₂—NHCO), 2.80-2.73 (m, 2H, CH₂NH₃⁺), 1.54-1.48 (m, 2H, CH₂), 1.46-1.40 (m, 2H, CH₂), 1.32-1.25 (m, 4H, CH₂) ppm.

¹³C{¹H}-NMR (126 MHz, DMSO) δ=161.8 (q, J=1.3 Hz, CONH), 158.1 (q, J=33.0 Hz, O₂CCF₃), 155.7 (q, J=5.1 Hz, CCF₃), 143.6 (d, J=0.8 Hz, HC═CH), 142.5 (s, HC═CH), 141.0 (q, J=35.6 Hz, CC═O), 122.5 (q, J=268.1 Hz, CCF₃), 85.2 (s, CH—O), 82.4 (q, J=2.1 Hz, CH—O), 38.8 (s, CH₂NHCO), 38.5 (s, CH₂NH₃⁺), 28.6 (s, CH₂CH₂CH₂), 26.9 (s, CH₂CH₂CH₂), 25.7 (s, CH₂CH₂CH₂) 25.4 (s, CH₂CH₂CH₂) ppm.

TABLE 1
The chemical formulas of the compounds mentioned above
are presented in this Table.

| | |
|---|---|
| Compound 1 | 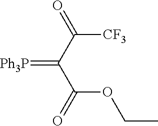 |
| Compound 2 | 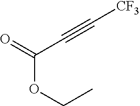 |
| Compound 3 | 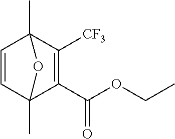 [SMILES: CCOC(=O)C1=C(C(F)(F)F)C2(C)C=CC1(C)O2] |
| Compound 4 | 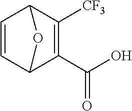 [SMILES: O=C(O)C1=C(C(F)(F)F)C2C=CC1O2] |
| Compound 5 | 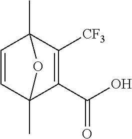 [SMILES: CC12C=CC(C)(O1)C(C(F)(F)F)=C2C(=O)O] |
| Compound 6 | 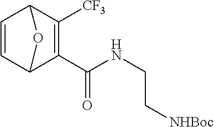 [SMILES: CC(C)(C)OC(=O)NCCNC(=O)C1=C(C(F)(F)F)C2C=CC1O2] |
| Compound 7 | 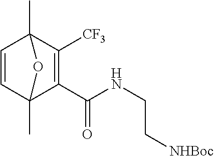 [SMILES: CC(C)(C)OC(=O)NCCNC(=O)C1=C(C(F)(F)F)C2(C)C=CC1(C)O2] |
| Compound 8 | 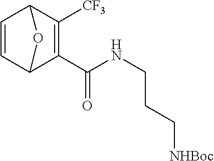 [SMILES: CC(C)(C)OC(=O)NCCCNC(=O)C1=C(C(F)(F)F)C2C=CC1O2] |
| Compound 9 | 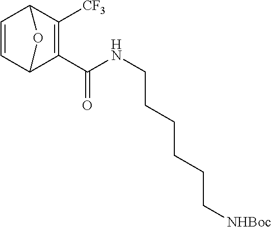 [SMILES: CC(C)(C)OC(=O)NCCCCCCNC(=O)C1=C(C(F)(F)F)C2C=CC1O2] |
| Compound 10 | 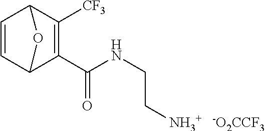 |
| Compound 11 | 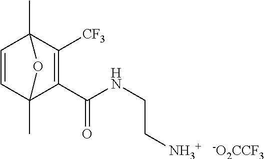 |
| Compound 12 | 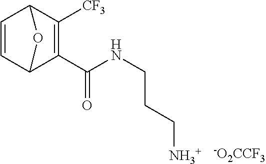 |
| Compound 13 | 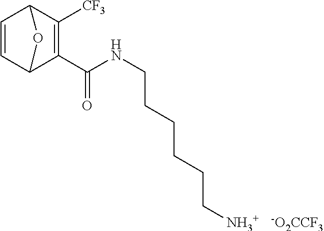 |

### General Procedures

### Carboxymethylation of Biopolymers:

The desired biopolymer (e.g. pullulan (25 g)) was dissolved in deionized water (375 mL). Subsequently, 8 M aq. NaOH (125 mL) and chloroacetic acid (50.1 g, 0.53 mol) were added and the reaction mixture was heated to 62° C. After 90 min, the solution was adjusted to pH 6.5 with 6 M aq. HCl and poured into distilled methanol (3 L). The resulting precipitate was filtered off and the residue was dried at 40° C. and 20 mbar. The carboxymethylated biopolymer was obtained as a white solid.

A higher degree of substitution was achieved by subjecting the obtained product to repetitive carboxymethylation reactions. The number of repetitive cycles is described by using a CM suffix (e.g. CM3 corresponds to 3 repetitive carboxymethylation cycles).

### Two Component System (Adding Linker Unit to Carboxymethylated Biopolymer):

A desired biopolymer (dextran 250 kDa CM6, 100 mg, 0.50 mmol) was dissolved in 0.025 M MES buffer (50 mL). The desired linker unit NH₂—(CH₂CH₂O)ₛ—CH₂CH₂N₃ with s=3 (98.15 μL, 0.50 mmol), EDC-HCl (0.948 g, 4.95 mmol) and NHS (56.93 mg, 0.50 mmol) were added sequentially. The reaction was stirred at RT for 2.5 days and then transferred into a dialysis tube (cut-off: 14 kDa). The latter was layed into a 5 L beaker containing an aqueous deionized NaCl solution and dialyzed for 4 days with decreasing NaCl concentration (day 1: 20 g/L, day 2: 10 g/L, and day 3-4: 0 g/L). Each day, the aqueous deionized NaCl solution was renewed three times. Subsequently, the dialyzed solution was filtered through absorbent cotton and freeze-dried. The modified biopolymer could be isolated as a cotton wool-like solid (87.80 mg).

### Hydrogel Gelation Reaction:

The resulting modified biopolymer chains (e.g. pullulan, dextran, alginate and hyaluronan) with different functional groups (degrees of substitutions are variable) were dissolved in an appropriate solvent (e.g. deionized water, buffer, organic solvent mixtures) and the two components (according to FIG. 2) were mixed. The gelation proceeded under mild conditions via a thermo-induced copper-free “click”-reaction (40° C., over night), resulting in the irreversible formation of a covalently crosslinked hydrogel, which contains triazole units connecting the two biopolymer chains.

### NMR Analysis:

The ¹H and ¹³C and ¹⁹F NMR spectra were measured on BrukerAVANCE-400/500/600 and DPX-200/400 spectrometers at room temperature and in the indicated deuterated solvents. The residual proton signal of the solvent (CDCl₃: δ (¹H-NMR)=7.26 ppm, D₂O: δ (¹H-NMR)=4.79 ppm und DMSO: δ (¹H-NMR)=2.50 ppm) served as a reference and for calibration of the ¹H-NMR spectra. The ¹³C-NMR spectra were recorded broadband decoupled and also calibrated to the solvent signal (CDCl₃: (¹³C-NMR)=77.2 ppm, and DMSO: (¹³C-NMR)=39.5 ppm). For the ¹⁹F-NMR, based quantification, trifluoroacetic acid methyl ester was used as an internal standard. Samples in deuterated water were measured using a water suppression method.

Coupling constants J were expressed in Hz and chemical shifts in ppm. The signal multiplicities were abbreviated as follows for simplicity: singlet (s), duplet (d), triplet (t), quartet (q), and multiplet (m).

### High-Resolution Mass Spectroscopy (HR-MS):

High-resolution mass spectra were measured on a Water Aquity UPLC system with a QTof Premier detector (ESI and APCI-MS/MS) or on a Water Allicance 2695 with Micromass LCTPremier detector. Samples were dissolved in water, acetonitrile, or methanol and injected using either an HPLC system or a direct inlet. The measured values are given in mass/charge (m/z).

### Column Chromatography:

Manual column chromatography was carried out using overpressure. For this purpose, silica gel (particle size: 40-63 μm, normal phase) from Macherey-Nagel and indicated running medium mixtures (e.g. PE/EtOAc 1:1) were used. Thin layer chromatography (DC silica gel 60 F254 glass plates) from Merck (pore size: 60 Å, layer thickness: 210-270 μm, fluorescent indicator UV254) was used to detect the products.

### SEM:

The morphological investigations of the hydrogel network were performed using scanning electron microscopes (FEI Nova 600 FEG und FEI NOVA 200 NanoLAB) with additional focused ion beam. For this purpose, the liquid hydrogel samples were drop and spin coated onto small silicon chip surfaces and polymerized to a hydrogel at 40° C. overnight. The samples were then coated with a 5 nm thick layer of elementary carbon using LEICA EMACE600 before they were examined by SEM.

### Swelling Ability:

After successful gelation, the hydrogels were frozen to −20° C. and then freeze-dried. Deionized water/buffer was added to the freeze-dried hydrogel (m₀) for maximum swelling. The excess of liquid was removed and the swollen hydrogel was weighed (m₁).

The swelling ratio were calculated as followed:

$$swelling rate = \frac{{m}_{1} - {m}_{0}}{{m}_{0}} \cdot 100 \%$$

### Rheological Measurements of Polysaccharides:

The rheological properties of the polysaccharides were studied using the rheometer MCR302 from Anton Paar. The gelation rate of the hydrogels at 40° C. was investigated by using a PP20-SN33813 system with a normal force of 0 N, a frequency of 1 Hz, an amplitude γ=0.1% and a gap size of 1 mm. The sample was prepared as follows: The resulting modified biopolymer chains (e.g. pullulan, dextran, alginate and hyaluronan) with different functional groups (degrees of substitutions are variable) were dissolved in an appropriate solvent (e.g. deionized water, buffer, organic solvent mixtures) and the two components (according to FIG. 2) were mixed. The gelation reaction took place during the measurement, as indicated by the increased storage module.

### Viscosity Measurements of Polysaccharides:

The viscosity properties of the polysaccharides were studied using the rheometer MCR302 from Anton Paar. The viscosity measurements were investigated by using a PP20-SN33813 system with a normal force of 0 N, an amplitude γ=0.1, 100% log, [slope]=10 points/decade, a given shear rate (\dot{γ}) from 10 s⁻¹ to 10,000 s⁻¹, a temperature of 19.3° C. and a gap size of 1 mm. The sample was prepared as follows: The resulting modified biopolymer chains (e.g. pullulan, dextran, alginate and hyaluronan) with different functional groups (degrees of substitutions are variable) were dissolved in an appropriate solvent (e.g. deionized water, buffer, organic solvent mixtures) and analyzed in the rheometer.

### Determination of Enzyme Activity Via a Colorimetric Assay:

The resulting modified biopolymer chains (e.g. pullulan, dextran, alginate and hyaluronan) with different functional groups (degrees of substitutions are variable) were dissolved in an appropriate solvent (e.g. deionized water, buffer, organic solvent mixtures) and the two components (according to FIG. 2) were mixed.

To this mixture, a certain concentration of enzyme was added. The gelation was conducted in a microtiter plate with a volume of 25 μl by incubation at 40° C. over night. Afterwards, the hydrogel samples were washed with deionized water by shaking for 30 mins at 50 rpm and the excess of deionized water was removed. To all standards, blanks, and samples the specified reaction mixture (see Table 1) was added and the fluorescence (Excitation: 530/13, Emission: 590/18; AmplexRed®) was measured with a Cytation5 from BioTek. Measurement was conducted at a constant temperature of 37° C.

TABLE 2
Composition of the reaction mixtures of the AmplexRed ® Assay.

| glucose oxidase | uricase |
|---|---|
| 100 mM D-glucose | 1.0 mM uric acid |
| 0.2 U/mL horseradish peroxidase | 0.4 U/mL horseradish peroxidase |
| 100 μM Amplex ® Red reagent | 100 μM Amplex ® Red reagent |
| in 50 mM phosphate | in 50 mM phosphate |
| buffer (pH 7.4) | buffer (pH 7.4) |

* final concentration in well will be divided by two (25 μl sample, 25 μl assay reaction mix)

### Fourier-Transform Infrared Spectroscopy:

Infrared spectra (IR) were recorded on a Shimadzu ATR-FT-IR spectrometer. All biopolymer samples were measured as freeze-dried lyophilizate.

### Atomic Forced Microscopy:

Bruker Dimension ICON was used to analyze the surface geometry of the hydrogels.

### Conductive Titration:

The degree of substitution of the biopolymers was determined by conductive titration with a TitroLine®7000.

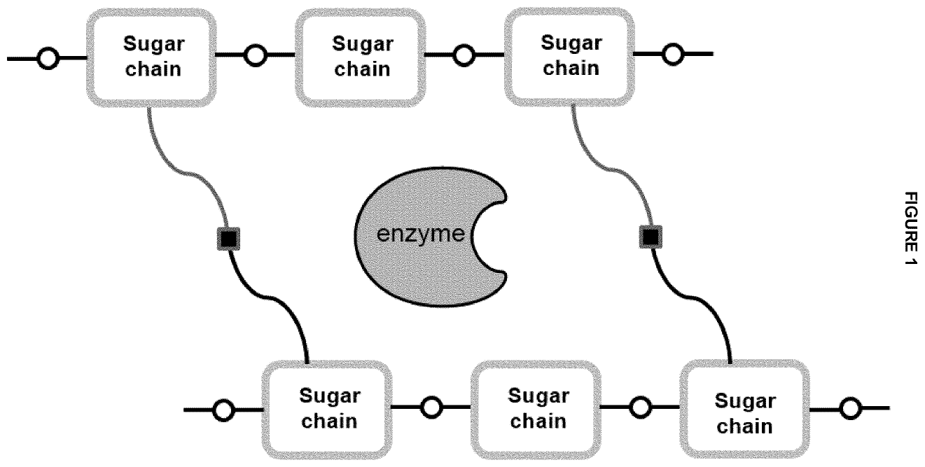

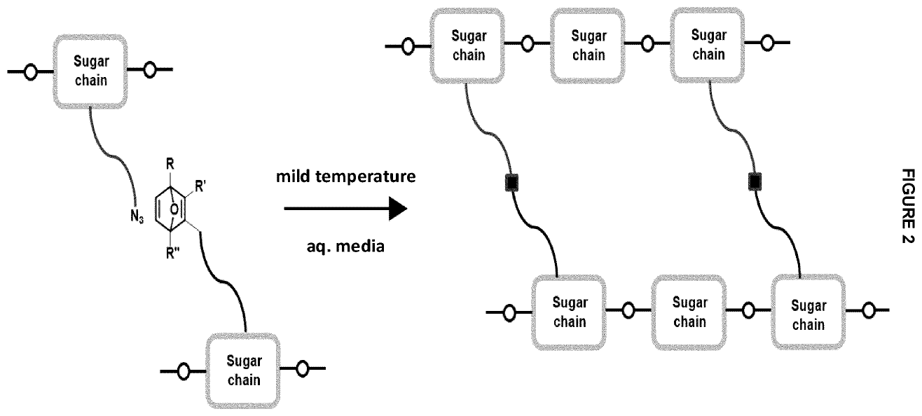

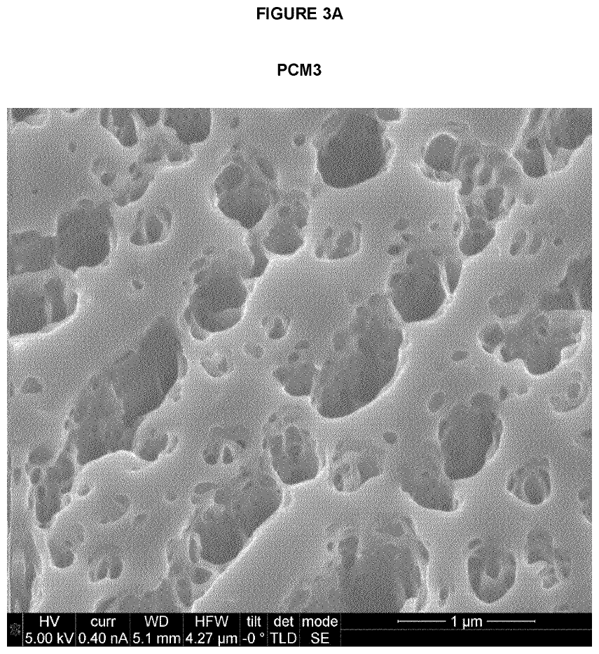

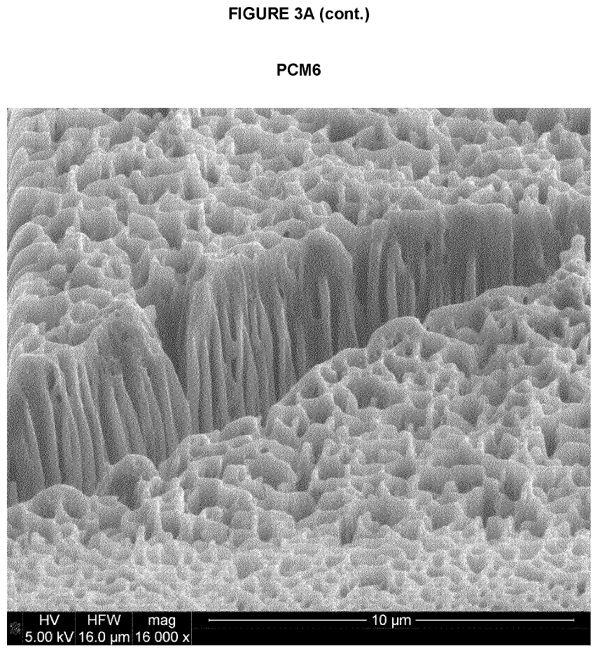

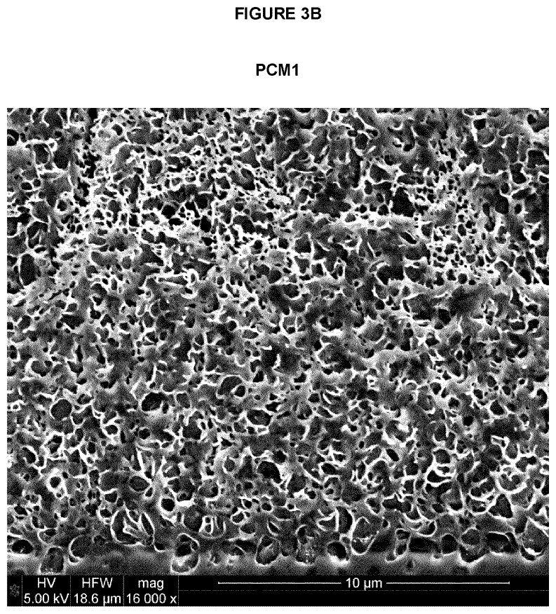

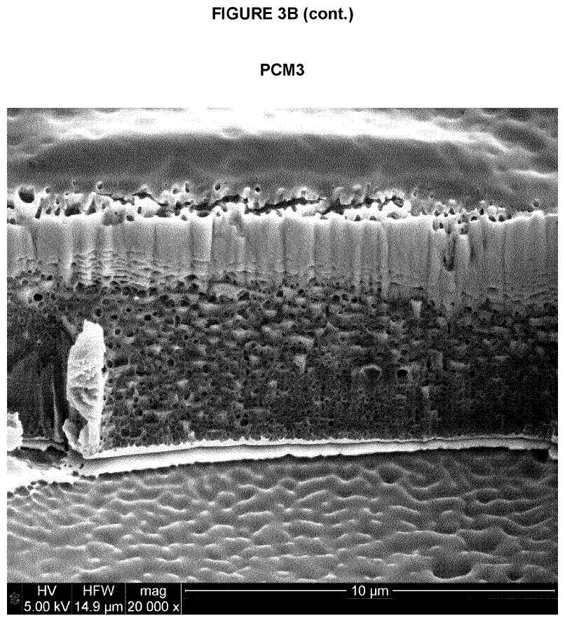

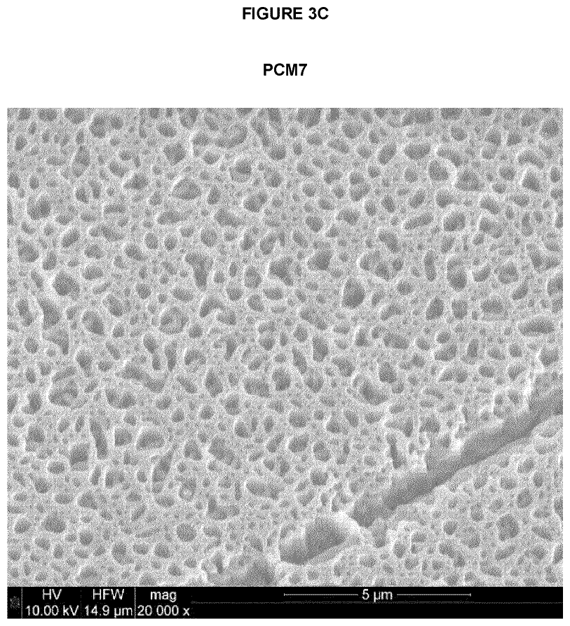

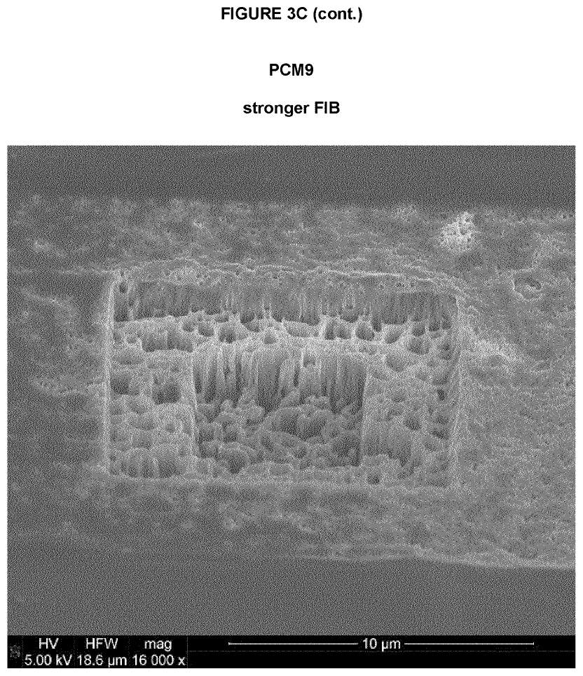

A two component system (crosslinked polysaccharides with three degrees of variability) according to FIG. 1 is provided: Two modified biopolymer chains (e.g. pullulan, dextran, lentinan, alginate and hyaluronan) with reactive functional groups (one biopolymer carrying azide residues, the other biopolymer carrying oxanorbornadiene derivatives; degrees of substitutions are variable) form a triazole via thermo-induced copper-free “click”-reaction, which is described in detail with concrete examples in the following:

### Example 1: Carboxymethylation of Dextran

Dextran (500 kDa, 10.00 g, 0.06 mol) was dissolved in deionized water (150 mL). Subsequently, 8 M aq. NaOH (50 mL) and chloroacetic acid (20.40 g, 0.22 mol) were added and the reaction mixture was heated to 62° C. After 90 min, the solution was adjusted to pH 6.5 with 6 M aq. HCl and poured into distilled methanol (1.4 L). The resulting precipitate was filtered off and the residue was dried at 40° C. and 20 mbar. The carboxymethylated biopolymer was obtained as a white solid (12.34 g).

A higher degree of substitution was achieved by subjecting the obtained product to repetitive carboxymethylation (CM) reactions.

The following examples were obtained in a similar fashion as described above for Example 1:

TABLE 3
Carboxymethylated biopolymers.

| | Dextran | | Dextran | | Dextran | | Dextran | | Dextran | |
|---|---|---|---|---|---|---|---|---|---|---|
| | 10 kDa | | 70 kDa | | 150 kDa | | 250 kDa | | 500 kDa | |
| | scale | yield | scale | yield | scale | yield | scale | yield | scale | yield |
| CMX | [g] | [g] | [g] | [g] | [g] | [g] | [g] | [g] | [g] | [g] |
| CM1 | 15.00 | 15.35 | 15.00 | 17.95 | 10.00 | 8.83 | 10.00 | 7.01 | 10.00 | 12.34 |
| CM2 | 12.00 | 12.51 | 15.00 | 13.55 | 9.00 | 8.83 | 10.00 | 12.12 | 9.00 | 9.42 |
| CM3 | 12.00 | 9.85 | 13.50 | 12.08 | 6.00 | 5.90 | 9.00 | 9.00 | 6.00 | 6.05 |
| CM4 | 7.00 | 6.25 | 8.00 | 7.26 | 4.00 | 4.00 | 6.00 | 5.68 | 4.00 | 4.13 |
| CM5 | 5.80 | 5.47 | 5.80 | 6.48 | 2.00 | 1.64 | 3.00 | 2.83 | 2.00 | 1.72 |
| CM6 | | | | | | | 1.00 | 0.94 | 0.80 | 0.78 |

### Carboxymethylated Lentinan

Lentinan (400-800 kDa) CM1: scale: 8 g; yield: 3.49 g.

TABLE 4
Carboxymethylated pullulan.

Pullulan 100 kDa

| CMX | scale [g] | yield [g] |
|---|---|---|
| CM1 | 10.00 | 14.66 |
| CM2 | 13.00 | 13.16 |
| CM3 | 11.00 | 11.50 |
| CM4 | 10.00 | 9.67 |
| CM5 | 8.00 | 8.08 |
| CM6 | 5.00 | 4.85 |
| CM7 | 3.50 | 3.13 |
| CM8 | 2.00 | 1.85 |
| CM9 | 1.00 | 0.94 |

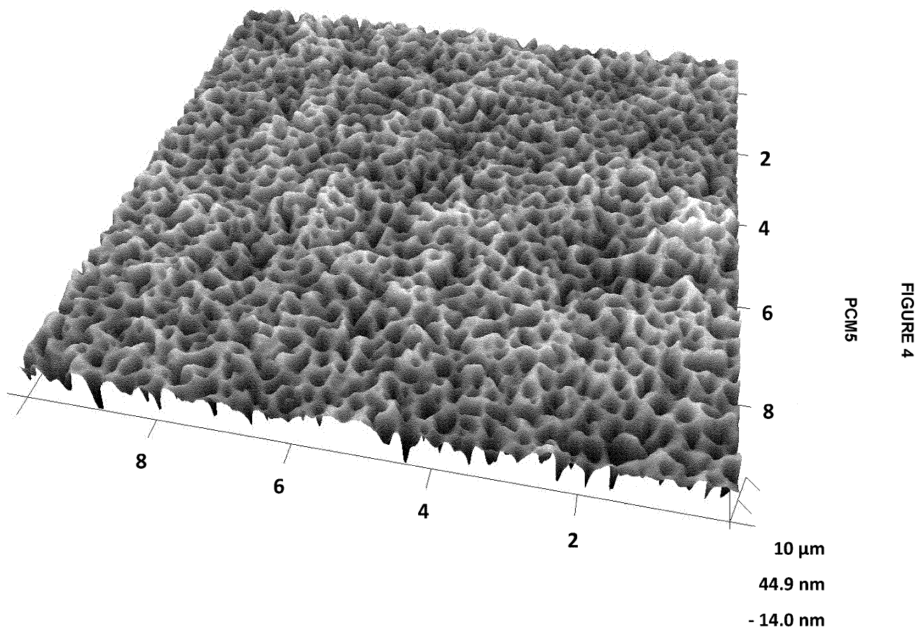

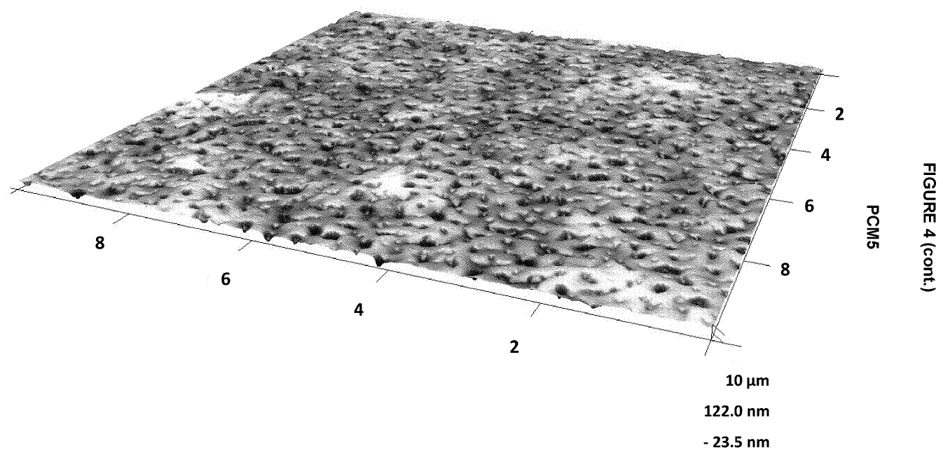

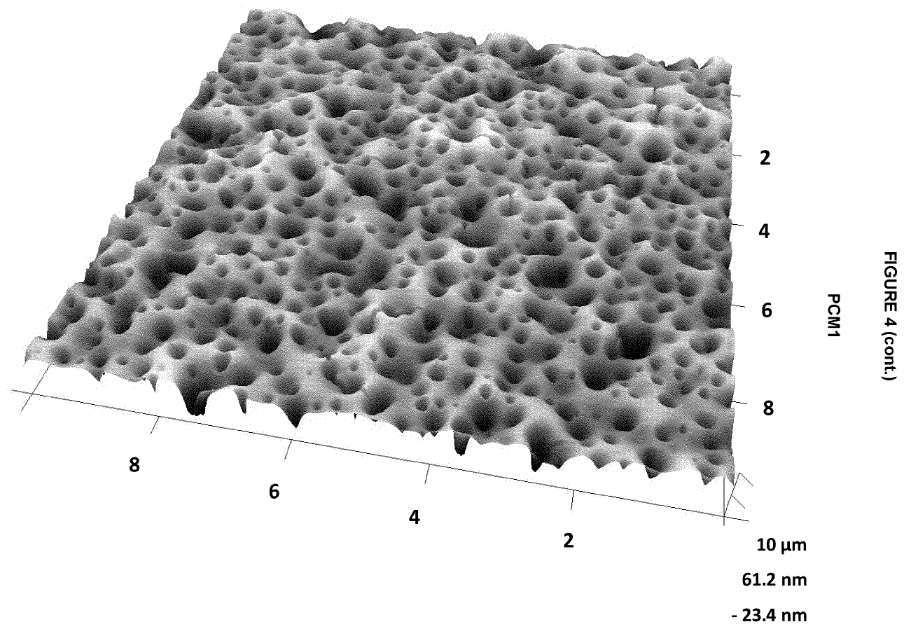

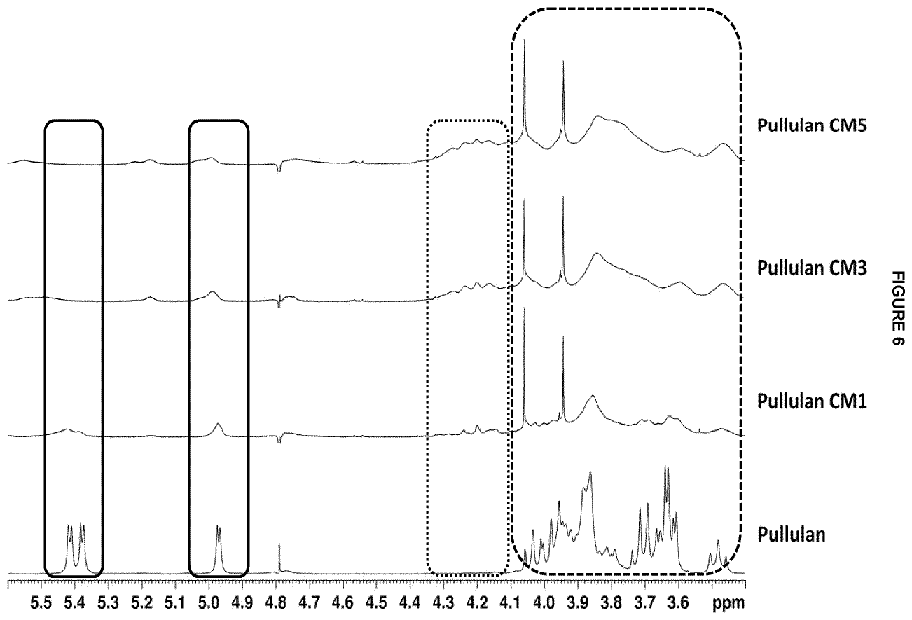

The degree of substitution of the obtained polysaccharides was characterized by FT-IR spectroscopy (see FIG. 5), ¹H-NMR spectroscopy (see FIG. 6) and conductive titration (see FIG. 9).

Both biopolymer chains are conjugated to functionalized spacers, e.g. a PEG-linker unit. The PEG-linker is variable in length and should be short (PEG(3)-PEG(25)). Different PEG-linker units (indicated in FIG. 2, see “R—N₃”) and carboxymethylated biopolymers lead to different pore sizes. The pore size will influence the activity of the enzyme and the diffusion behaviour of the substrate and of other molecules (see FIG. 1). In this context, the term “functionalized” refers to spacers carrying either an azide moiety or an oxanorbornadiene derivative Q, like described above (see scheme 1). Conjugation is achieved via amide coupling of carboxy groups of the biopolymer and amine groups in the linker.

### Example 2: Azide Functionalization of Dextran

A desired biopolymer (dextran, 250 kDa, CM4, 100 mg, 0.45 mmol) was dissolved in 0.025 M MES buffer (50 mL). The desired linker NH₂—(CH₂CH₂O)ₛ—CH₂CH₂N₃ with s=3, 90.12 μL, 0.45 mmol, EDC-HCl (0.87 g, 4.54 mmol) and NHS (52.27 mg, 0.45 mmol) were added sequentially. The reaction was stirred at RT for 2.5 days and then transferred into a dialysis tube (cut-off: 14 kDa). The latter was deposited in a 5 L beaker containing an aqueous deionized NaCl solution and dialyzed for 4 days with decreasing NaCl concentration (day 1: 20 g/L, day 2: 10 g/L, and day 3-4: 0 g/L). Each day, the aqueous deionized NaCl solution was renewed three times. Subsequently, the dialyzed solution was filtered through absorbent cotton and freeze-dried. The modified biopolymer could be isolated as a cotton wool-like solid (95 mg).

The following examples were obtained as described above:

TABLE 5
Introducing an azide unit (—NH—(CH₂CH₂O)ₛ—CH₂CH₂N₃ with s = 3) to a polysaccharide strand.

| | Dextran 10 kDa | | Dextran 70 kDa | | Dextran 250 kDa | | Dextran 500 kDa | |
|---|---|---|---|---|---|---|---|---|
| | scale | yield | scale | yield | scale | yield | scale | yield |
| CMX | [mg] | [mg] | [mg] | [mg] | [mg] | [mg] | [mg] | [mg] |
| CM2 | 100 | 41 | | | 200 | 226 | 200 | 193 |
| CM3 | | | | | 100 | 84 | 200 | 177 |
| CM4 | 100 | 93 | 100 | 58 | 100 | 95 | 200 | 172 |
| CM5 | | | | | 200 | 198 | 200 | 184 |
| CM6 | | | | | 100 | 107 | 100 | 74 |

Alginate (120-190 kDa): scale: 200 mg; yield: 172 mg.
Hyaluronan (70-80 kDa): scale: 100 mg; yield: 80 mg.
Lentinan CM1 (400-800 kDa): scale: 100 mg; yield: 74 mg.

TABLE 6
Introducing an azide unit (—NH—(CH₂CH₂
O)ₛ—CH₂CH₂N₃ with s = 3) to pullulan.

Pullulan 100 kDa

| CMX | scale [mg] | yield [mg] |
|---|---|---|
| CM1 | 200 | 158 |
| CM2 | 200 | 154 |
| CM3 | 200 | 205 |
| CM4 | 200 | 193 |
| CM5 | 200 | 198 |
| CM6 | 200 | 200 |
| CM7 | 200 | 206 |
| CM8 | 200 | 192 |
| CM9 | 200 | 234 |

Introducing an Azide Unit (—NH—(CH₂CH₂O)ₛ—CH₂CH₂N₃ with s=8) to Pullulan.
Pullulan (100 kDa) PCM5: scale: 50 mg; yield: 63 mg.
   Introducing an Azide Unit (—NH—(CH₂)ᵣ—N₃ with r=6) to Pullulan.
Pullulan (100 kDa) PCM1: scale: 150 mg; yield: 122 mg.
Pullulan (100 kDa) PCM5: scale: 150 mg; yield: 147 mg.
Alginate (120-190 kDa): scale: 200 mg; yield: 89 mg.

### Example 3: Oxanorbornadiene Functionalization of Dextran

A desired biopolymer (dextran, 250 kDa, CM4, 100 mg, 0.45 mmol) was dissolved in 0.025 M MES buffer (50 mL). The desired linker NH₂—(CH₂)ᵣ-Q with r=2, Q−1 (CF₃) and M/M′=H, 112.73 mg, 0.45 mmol, EDC-HCl (0.87 g, 4.54 mmol) and NHS (52.27 mg, 0.45 mmol) were added sequentially. The reaction was stirred at RT for 2.5 days and then transferred into a dialysis tube (cut-off: 14 kDa). The latter was deposited in a 5 L beaker containing an aqueous deionized NaCl solution and dialyzed for 4 days with decreasing NaCl concentration (day 1: 20 g/L, day 2: 10 g/L, and day 3-4: 0 g/L). Each day, the aqueous deionized NaCl solution was renewed three times. Subsequently, the dialyzed solution was filtered through absorbent cotton and freeze-dried. The modified biopolymer could be isolated as a cotton wool-like solid (88 mg).

The following examples were obtained as described above:

TABLE 7
Introducing an oxanorbornadiene unit (—NH—(CH₂)ᵣ-Q with r = 2,
Q-1 (CF₃) and M/M′ = H) to a polysaccharide strand.

| | |
|---|---|
| Dextran 250 kDa | Dextran 500 kDa |

| CMX | scale [mg] | yield [mg] | scale [mg] | yield [mg] |
|---|---|---|---|---|
| CM3 | | | 200 | 156 |
| CM4 | 100 | 88 | 100 | 94 |
| CM5 | 150 | 126 | 150 | 126 |

Alginate (120-190 kDa): scale: 200 mg; yield: 234 mg.
Hyaluronan (70-80 kDa): scale 100 mg; yield: 99 mg.
Lentinan CM1 (400-800 kDa): scale: 100 mg; yield: 65 mg.

TABLE 8
Introducing an oxanorbornadiene unit (—NH—(CH₂)ᵣ-Q with r =
2, Q-1 (CF₃) and M/M′ = H) to pullulan.

Pullulan 100 kDa

| CMX | scale [mg] | yield [mg] |
|---|---|---|
| CM1 | 200 | 183 |
| CM2 | 200 | 163 |
| CM3 | 200 | 210 |
| CM4 | 200 | 171 |
| CM5 | 200 | 201 |
| CM7 | 200 | 192 |
| CM8 | 167 | 211 |
| CM9 | 200 | 202 |

Introducing an Oxanorbornadiene Unit (—NH—(CH₂)ᵣ-Q with r=6, Q−1 (CF₃) and M/M′=H) to Pullulan.
Pullulan (100 kDa) PCM5: scale: 100 mg; yield: 122 mg.

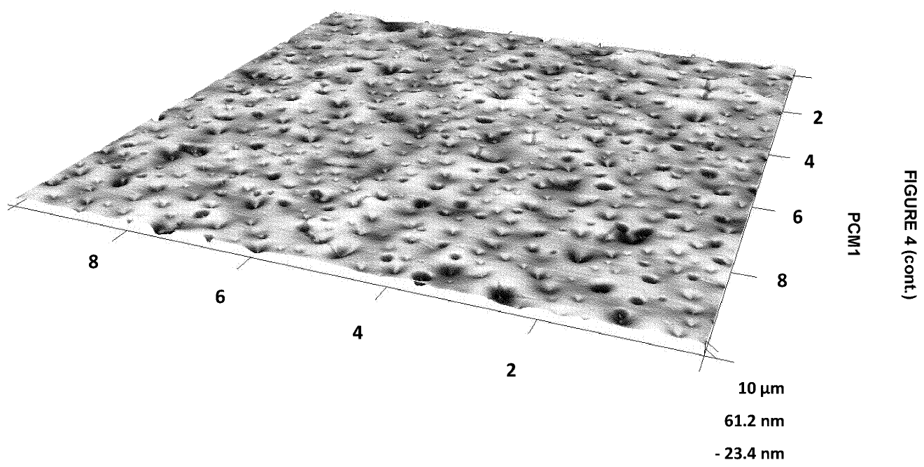

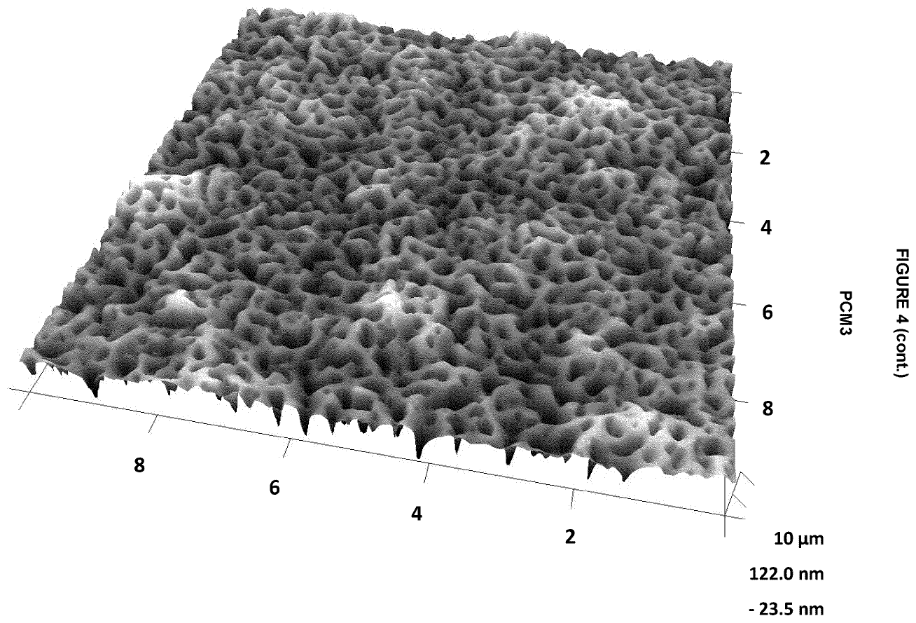

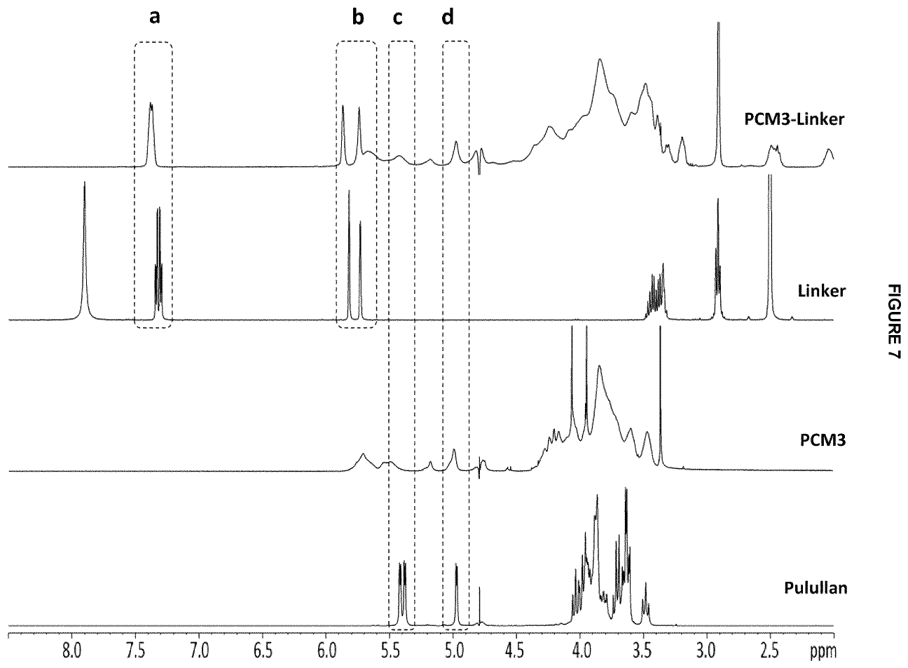

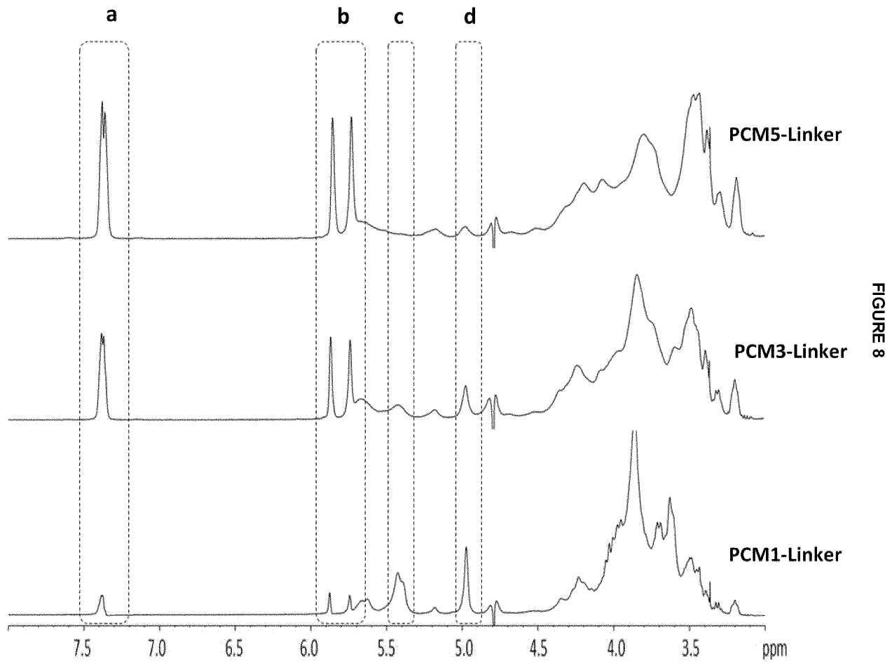

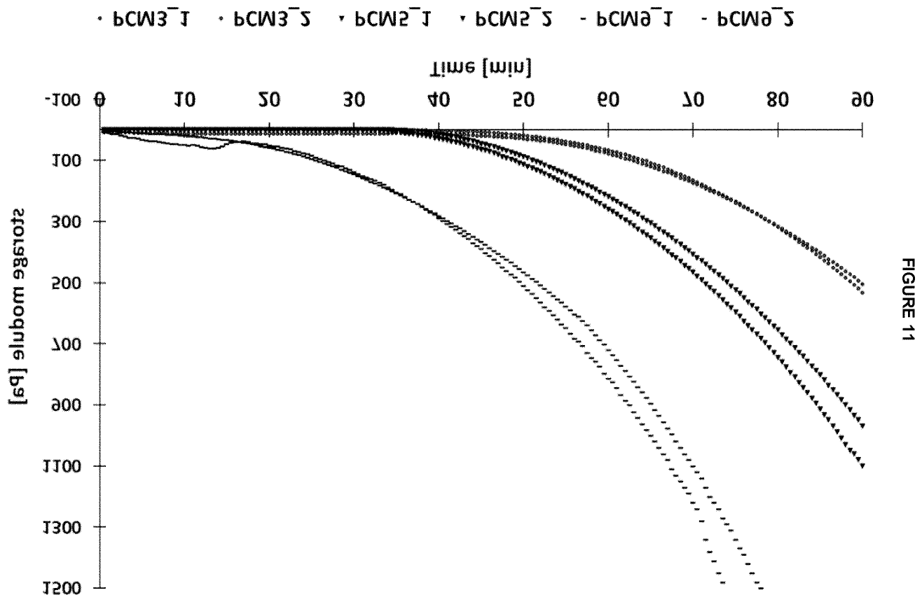

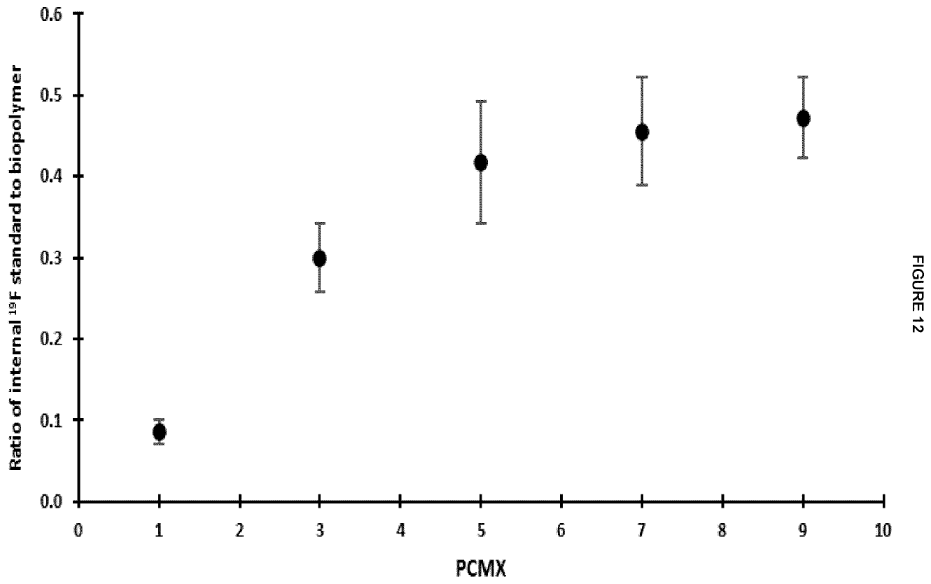

The obtained products can be analyzed by ¹H- and ¹⁹F-NMR spectroscopy (see e.g. FIGS. 7, 8 and 12). Specific IR-spectra of samples from different batches of one derivative are shown in FIG. 10.

### Example 4: Bio-Orthogonal 1,3-Dipolar Cycloaddition

The reaction (see FIG. 2) comprises: Crosslink via bio-orthogonal 1,3-dipolar cycloaddition, which is a copper-free “click”-reaction. To enable said reaction, the linker units linked to biopolymer of the Examples described above (azide-linked biopolymer and oxanorbornadiene-linked biopolymer, respectively) were mixed. Specific thermal gelation takes place under mild conditions (e.g. <40° C. in aqueous media). Mild reaction conditions are suitable for sensitive enzymes. The thus received irreversible network of sugar chains with immobilized enzyme provides high specificity and selectivity and bio-orthogonal material in a homogenous solution. Further, adjustment of viscosity can be achieved easily. Additionally, not crosslinked biopolymer and enzyme can be washed away. Gelation takes place after a certain time (of about 1 hour to 10 hours). Processing to different form factors, e.g. by printing or spin coating of the material is possible. Thus, the hydrogel according to the present invention allows to immobilize enzymes in a biocompatible network that does not require a covalent binding of the enzyme (see FIG. 2, wherein a=1-10 hours, depending on the material, mild reaction temperature and in aqueous media).

No side reactions occur and no toxic reagents (e.g. glutaraldehyde) are used. Very mild reaction conditions (40° C. in aqueous media) are applied as described above, while gelation time can be controlled by choice of material (substitution degree) and concentration of biopolymer (e.g. 10-40 mg/ml). Higher modified biopolymers (PCM5-PCM9) have a short gelation time (2-5 h; 40 mg/ml material). Low modified biopolymers (PCM1-3) have a gelation time from 3-10 h (40 mg/ml material).

### Example 5

The biocompatible hydrogel according to the present invention is a modular system, whose synthetic steps can be analyzed using various methods and can be manufactured in a well-defined manner.

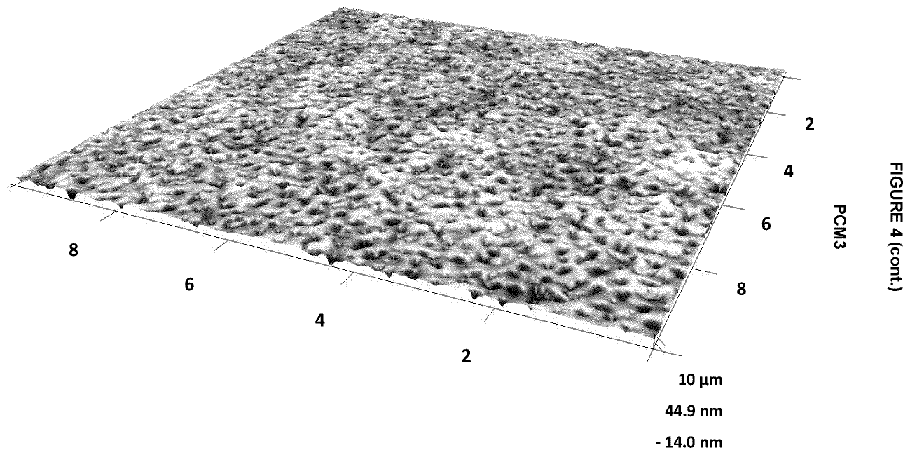

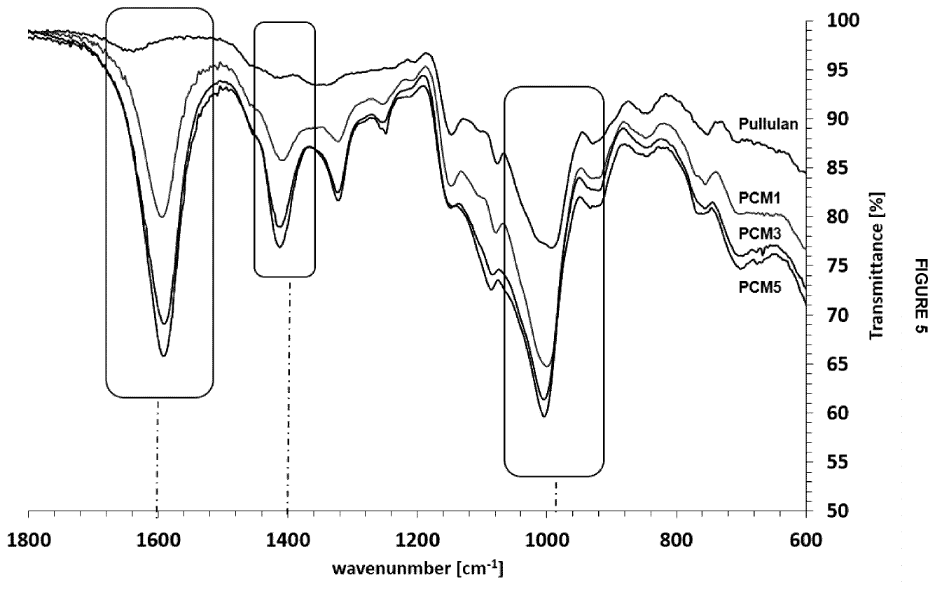

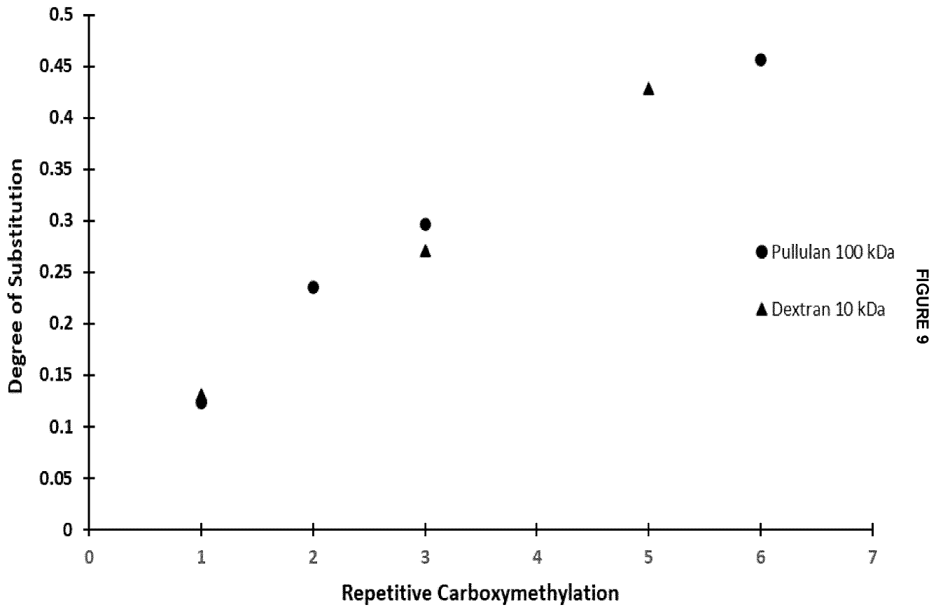

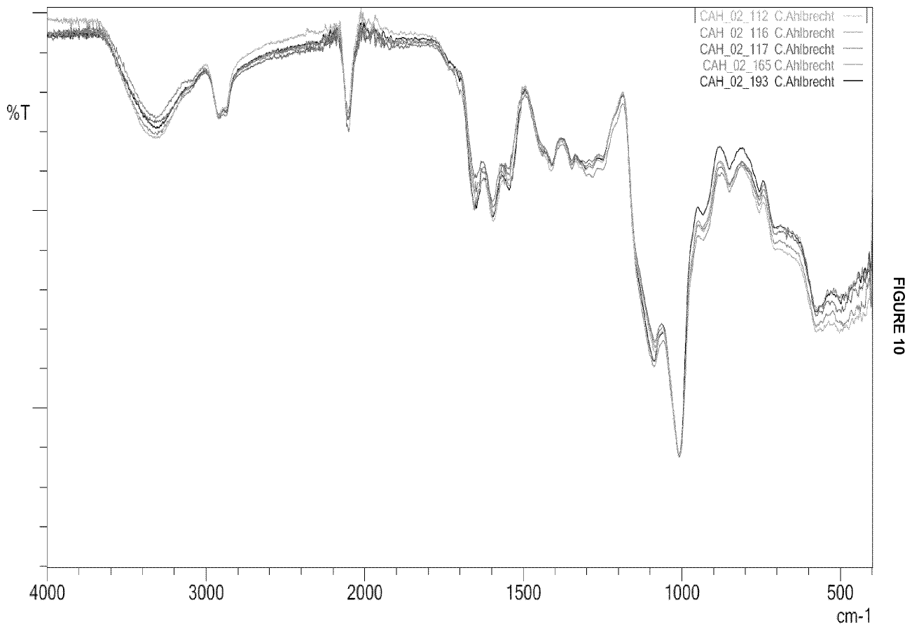

The repetitive carboxymethylation of a polysaccharide can be analyzed by conductive titration (see FIG. 7) and the degree of substitution rises with increasing carboxymethylation steps. Furthermore, the increase of substitution can be measured by FT-IR and ¹H-NMR. In the FT-IR spectra specific bands in the fingerprint region become more intense with a higher degree of substitution and an increasing number of functional groups (see FIG. 5). In the ¹H-NMR spectra, the decrease of the intensity of the signal of the anomeric proton was observed with increasing the introduced carboxymethyl-groups (see FIG. 6). Quantification by ¹⁹F-NMR showed the increased intensity of the signal of an introduced linker to differently modified polysaccharides. The number of introduced linker units correlates to the present carboxymethyl-groups in the polysaccharide (see FIG. 10). When investigating rheological properties in relation to gelation rate, it was shown that higher modified polysaccharides gelled faster due to their higher number of crosslinkable functional groups. A specific increase in the storage modulus over time was recorded for various materials (see FIG. 11). Moreover, five different batches of a five-fold repetitively modified pullulan carrying an azide linker showed similar FT-IR spectra (see FIG. 8).

### Example 6

### Investigation of the Swelling Rates in Hydrogels (Low Modified and Highly Modified Pullulan Hydrogels):

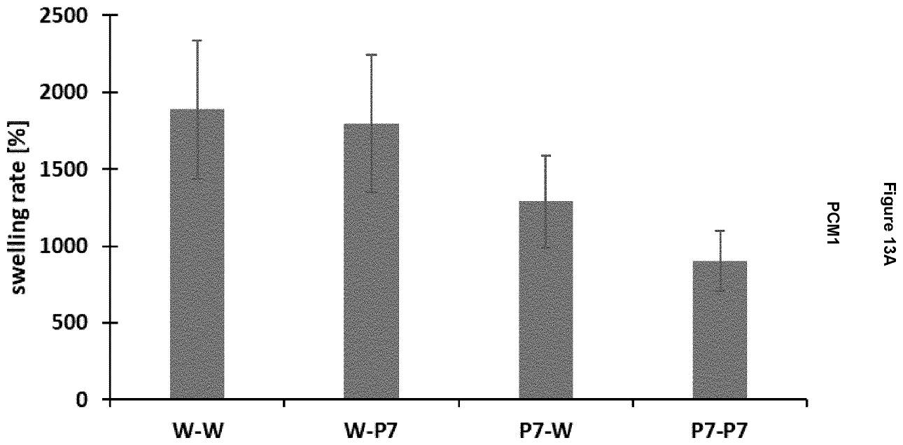

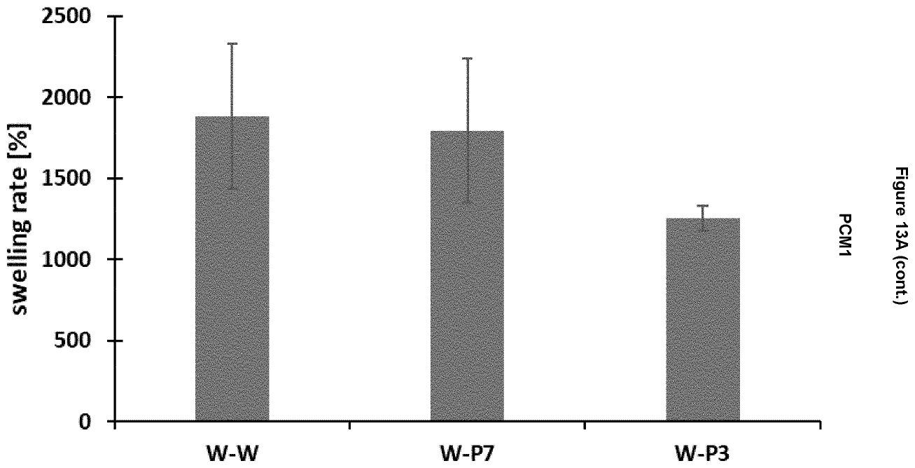

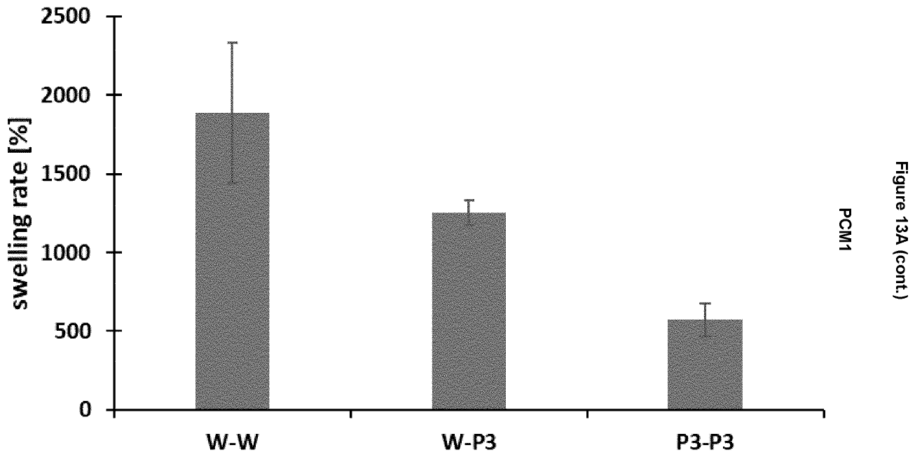

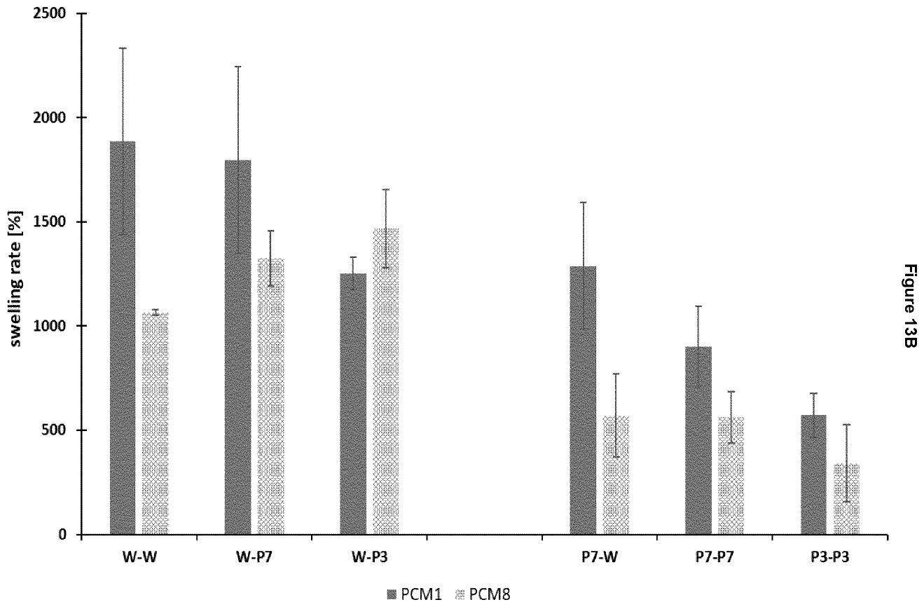

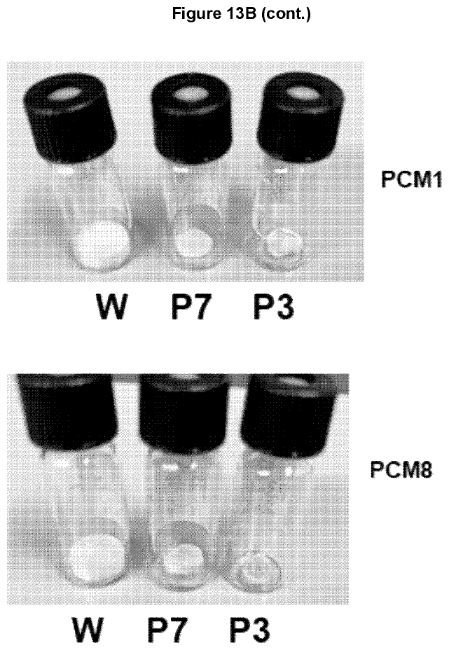

Investigation: To determine the influence of the used solvent (dH₂O or PBS buffer) on the gelation and the network formation as well as on the resulting pore sizes and to determine the change of the swelling rate when the lyophilized hydrogel is re-dissolved in the presence of salts at different pH values.
Results (see FIG. 13): Both shown hydrogels have the ability to swell more than 1000% in dH₂O. Low modified pullulan contains more free OH-groups and a highly porous structure. Water can enter easily and interact with the hydrophilic groups of the polysaccharide structure. PCM8: Higher degree of crosslinking leads to a denser hydrogel and to a decrease in water absorption.

In a low modified hydrogel (PCM1) the swelling rate increased with decreasing salt concentrations at physiological pH values. If the hydrogel was polymerized dH₂O, it showed the highest swelling rate. In contrast, the hydrogel (PCM1 and PCM8), which was gelled in PBS buffer at pH 3 and re-dissolved in PBS buffer at pH 3, showed a very low swellability.

### Example 7

Enzymes can be immobilized non-covalently in the hydrogel. After complete gelation, non-immobilized enzyme can be removed by washing.

Preparation: The resulting modified biopolymer chains (e.g. pullulan CM5) with different reactive functional groups (azide unit —NH—(CH₂CH₂O)ₛ—CH₂CH₂N₃ with s=3 to pullulan and —NH—(CH₂)ᵣ-Q with r=2, Q=Q−1 (CF₃) and M/M′=H to pullulan) were dissolved in phosphate buffer pH 7.4 (4 mg/100 μL each polysaccharide strand individually) and the two obtained components (according to FIG. 2) were mixed.

To this mixture, a certain concentration of an enzyme (e.g. glucose oxidase 100 mU/mL final concentration in well) was added. The gelation was conducted in a microtiter plate with a volume of 25 μL (each sample) by incubation at 40° C. over night. Afterwards, the hydrogel samples were washed with deionized water (25 μL) by shaking for 30 mins at 50 rpm and the excess of deionized water was removed. To all standards, blanks, and samples the specified reaction mixture (see Table 1) was added and the fluorescence was measured.

The example of immobilized uricase was obtained as described above. The reaction mixture was adjusted according to Table 1.

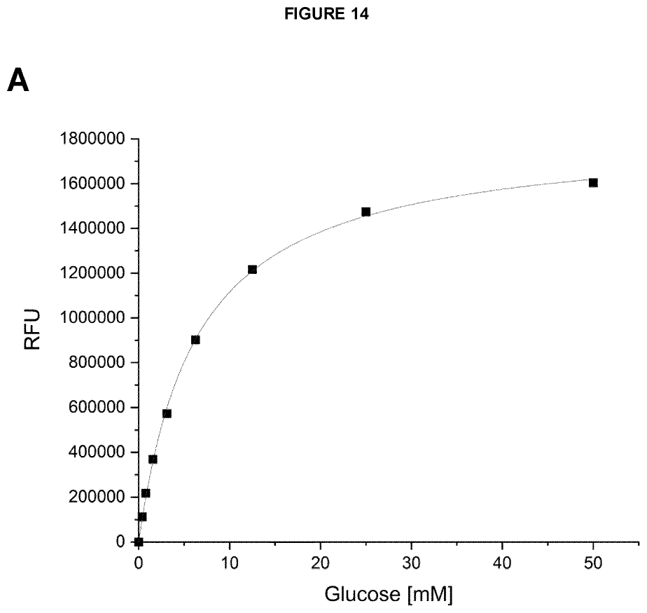

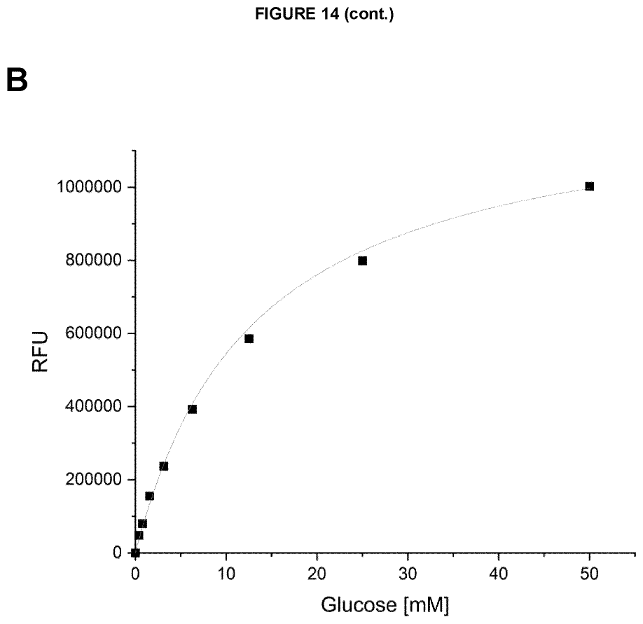

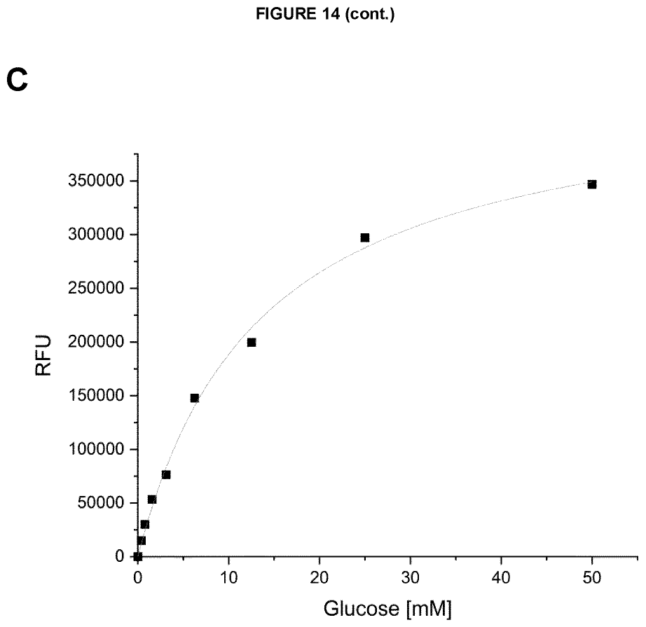

Results: Enzyme saturation curves could be recorded in different modified pullulan hydrogels (e.g. PCM2 and PCM8, meaning pullulan biopolymers obtained by running 2, respectively 8, carboxymethylation cycles). As a control, the enzyme was used in solution (see FIG. 14).

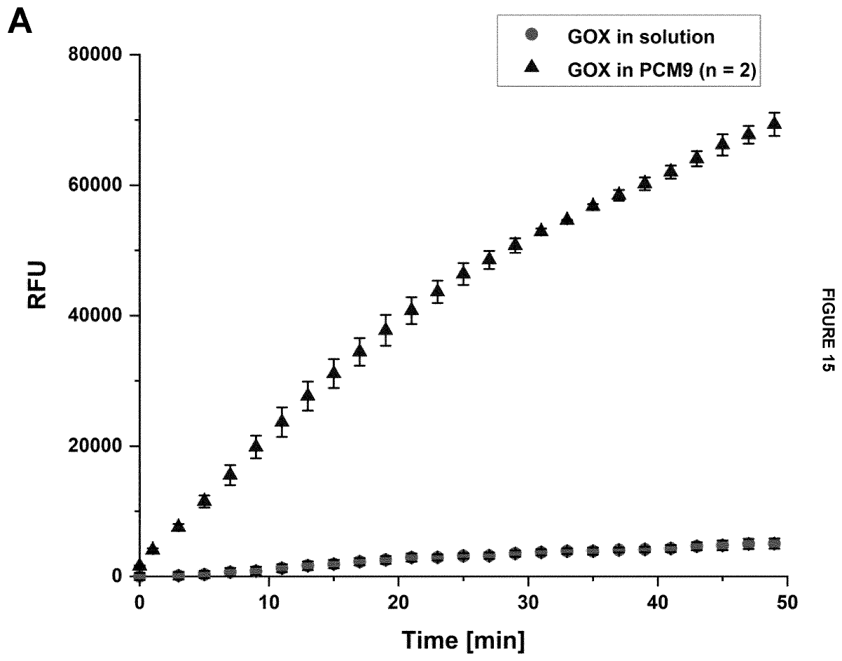

Long-term data of different hydrogel samples with immobilized glucose oxidase (FIGS. 15 and 16) were performed and the enzymatic activity was plotted on different days in comparison to the enzyme in solution as a control.

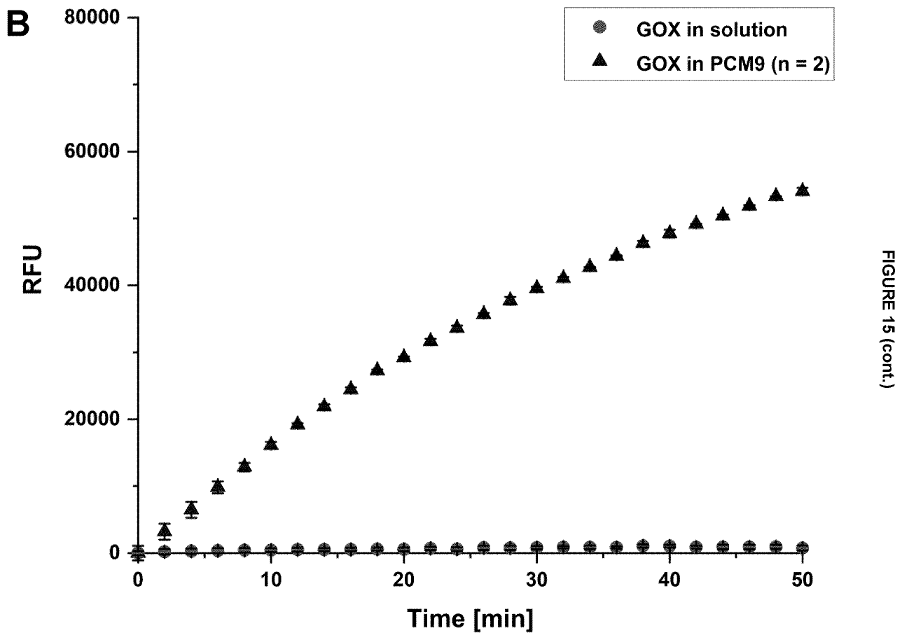

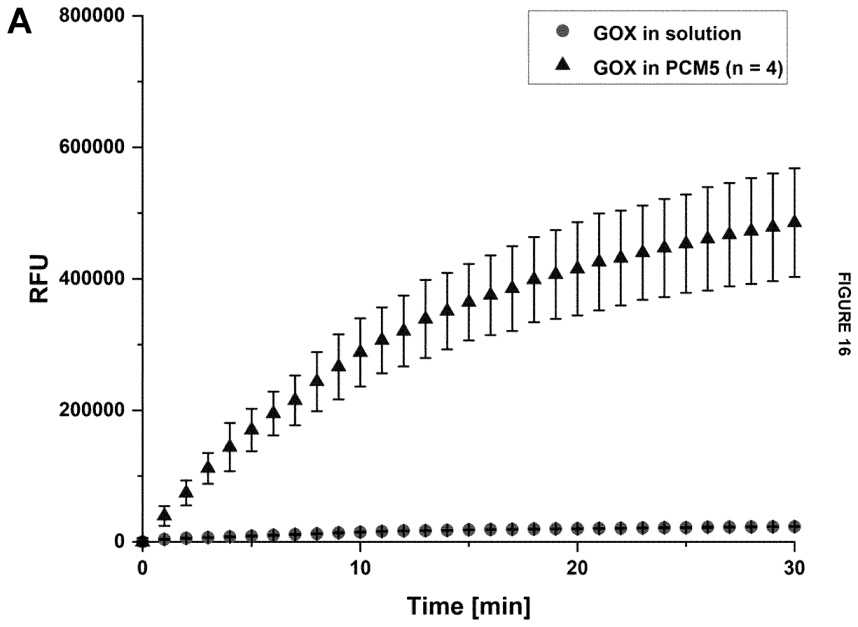

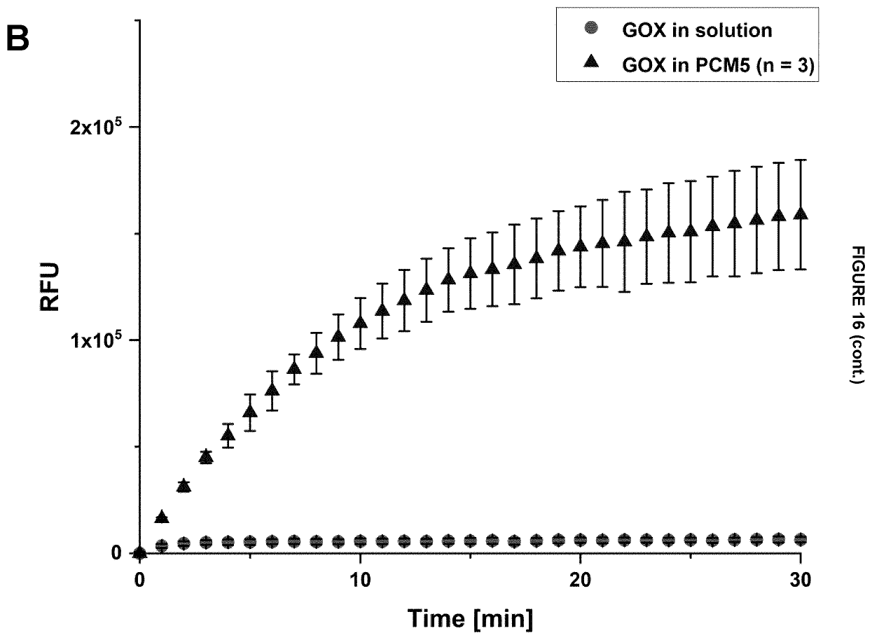

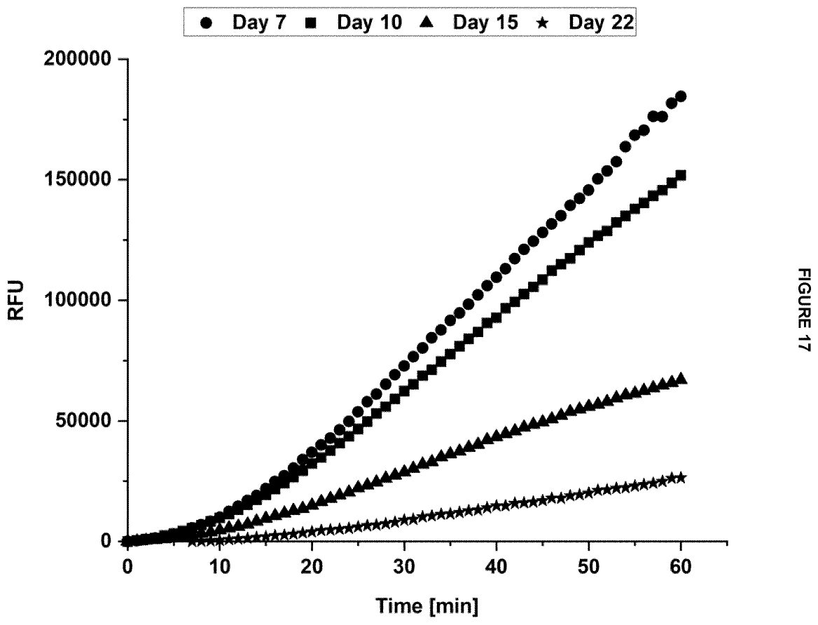

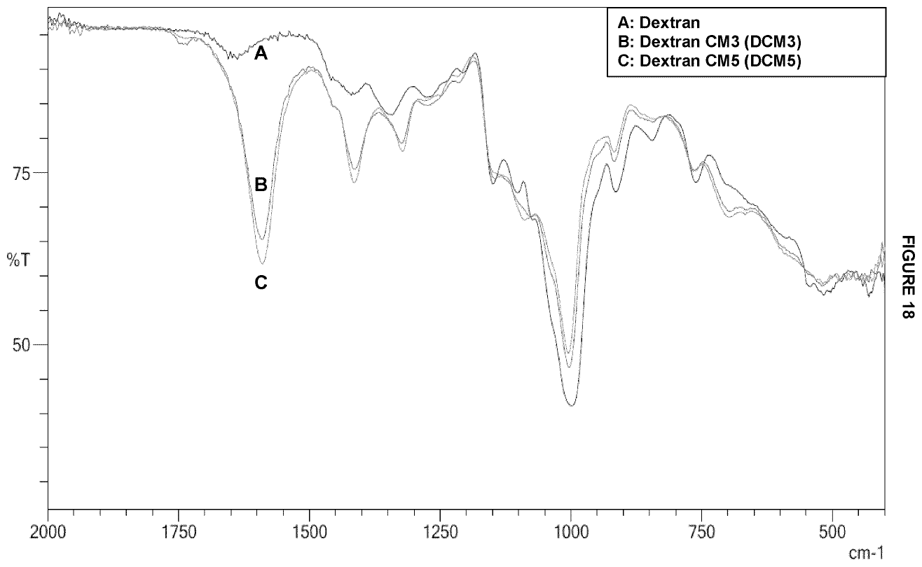

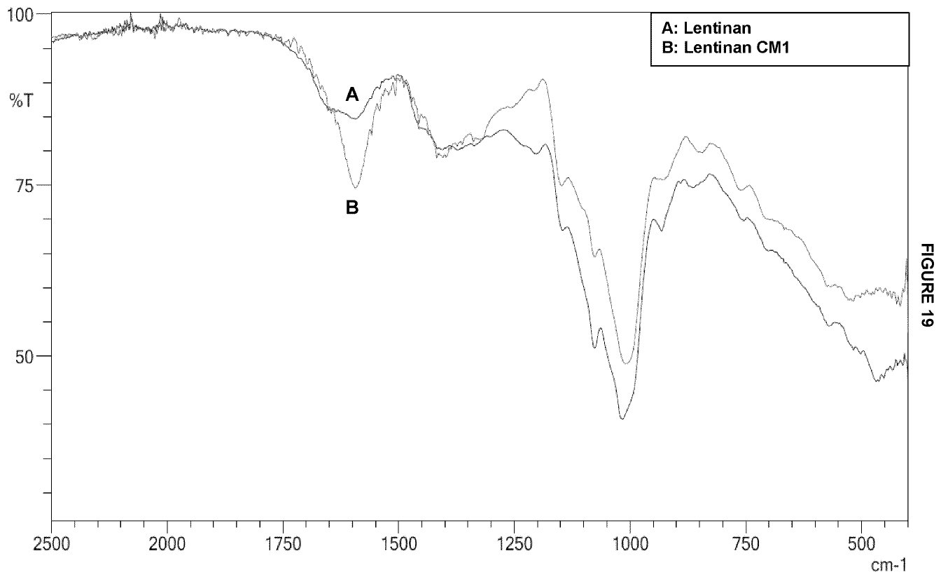

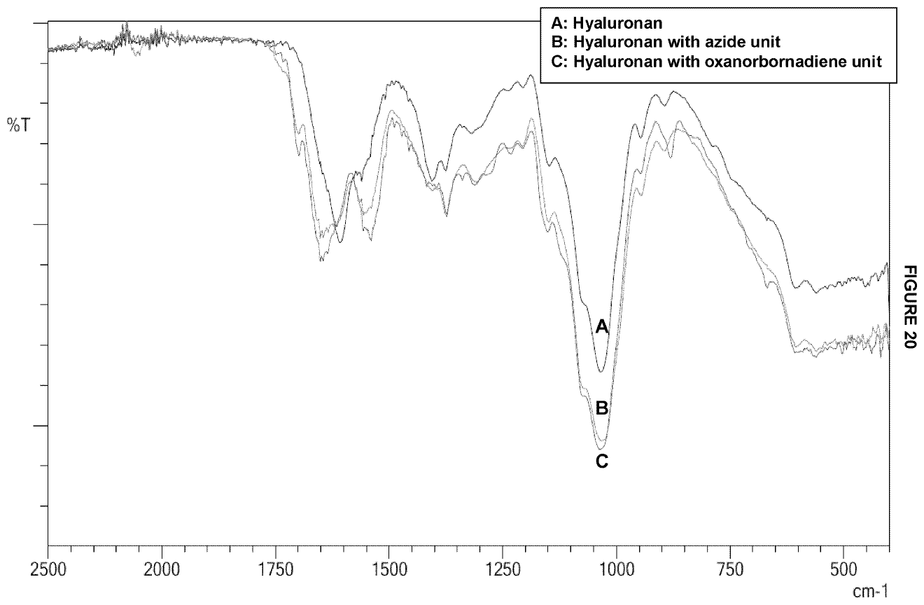

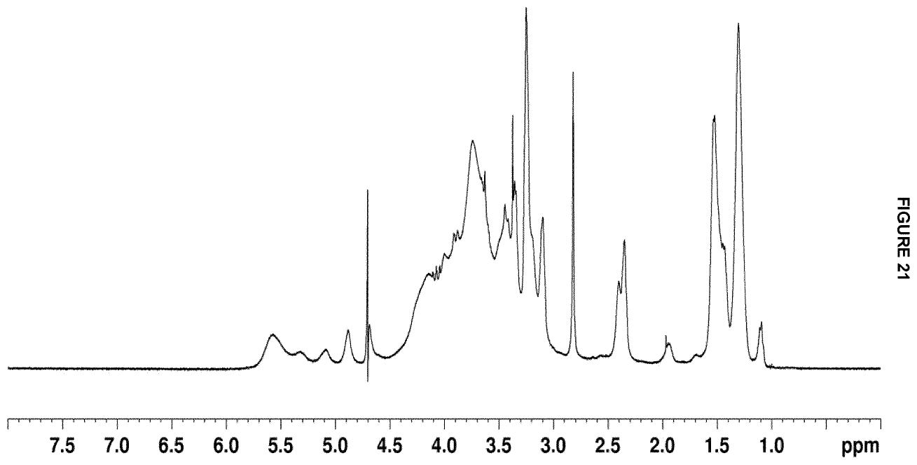

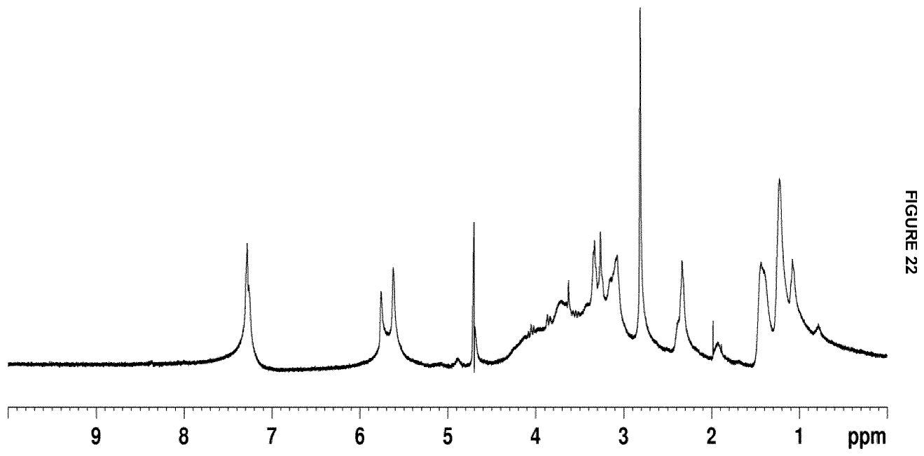

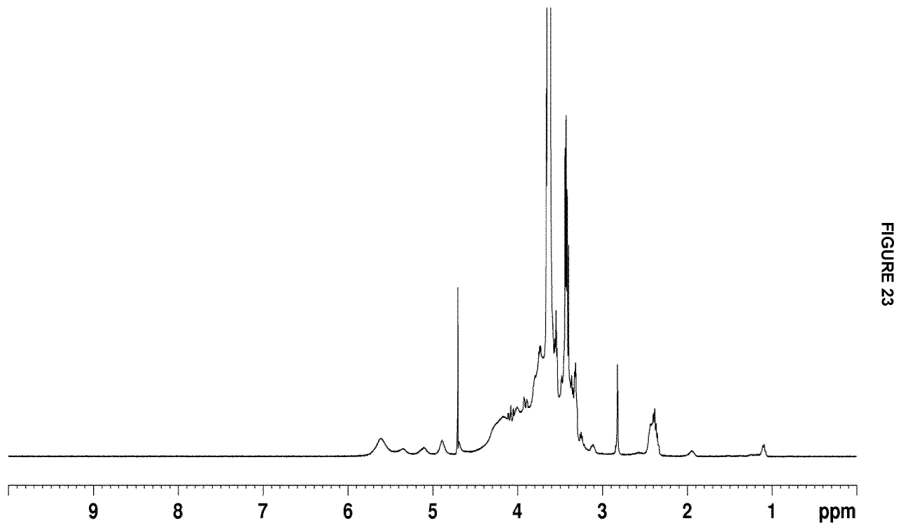

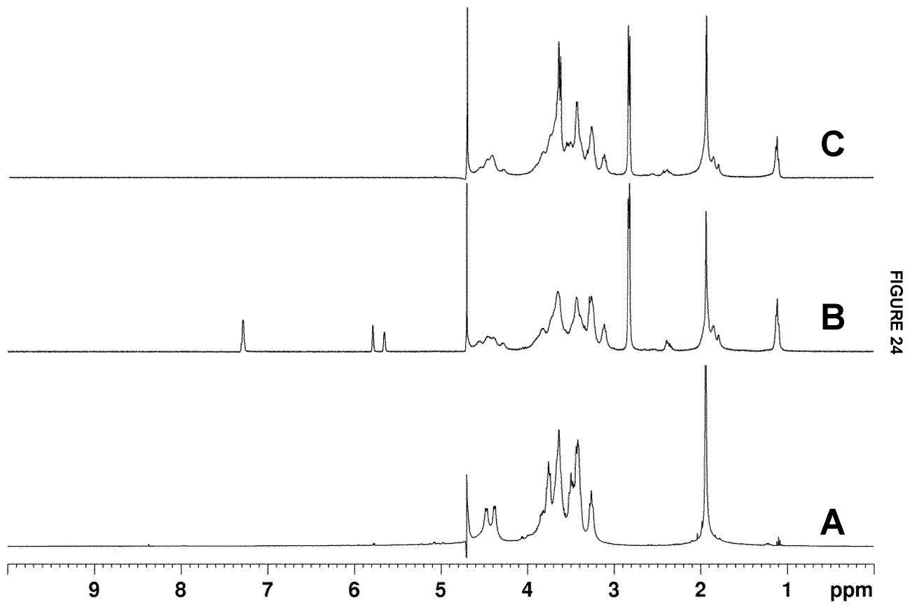

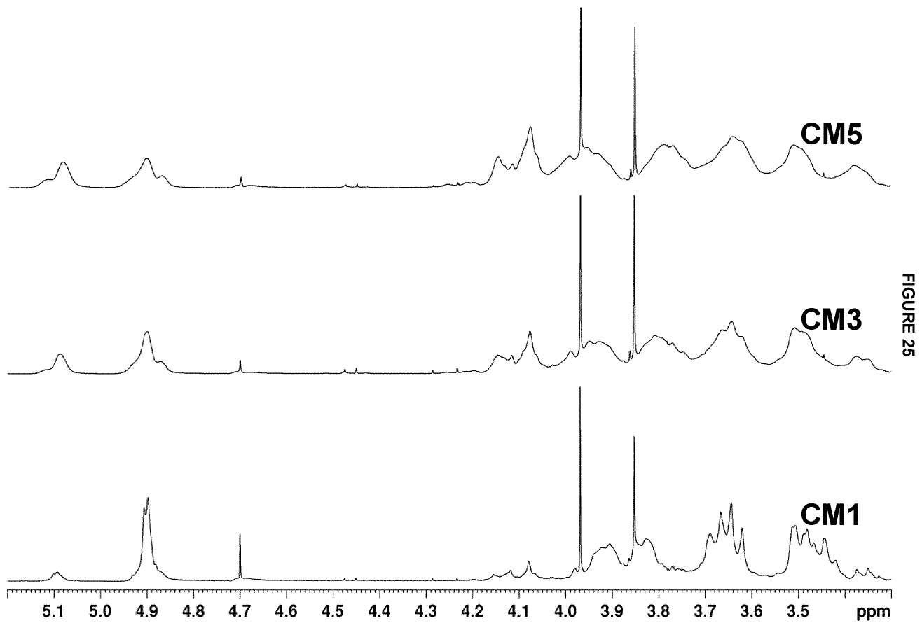

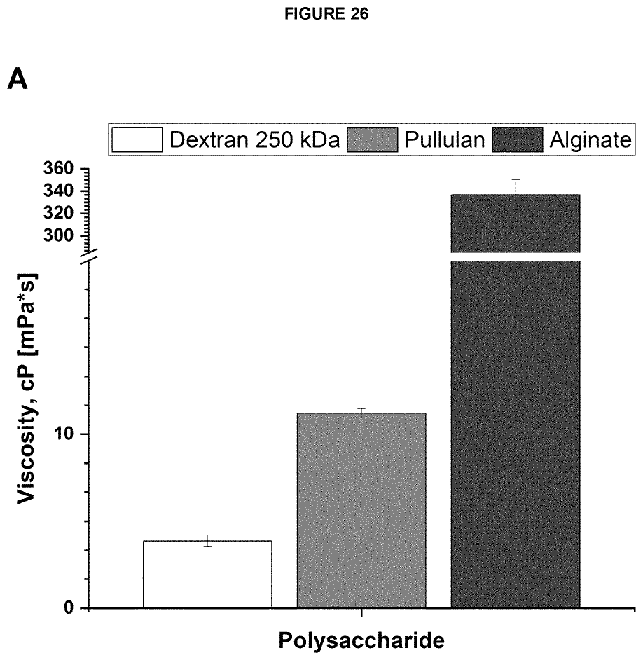

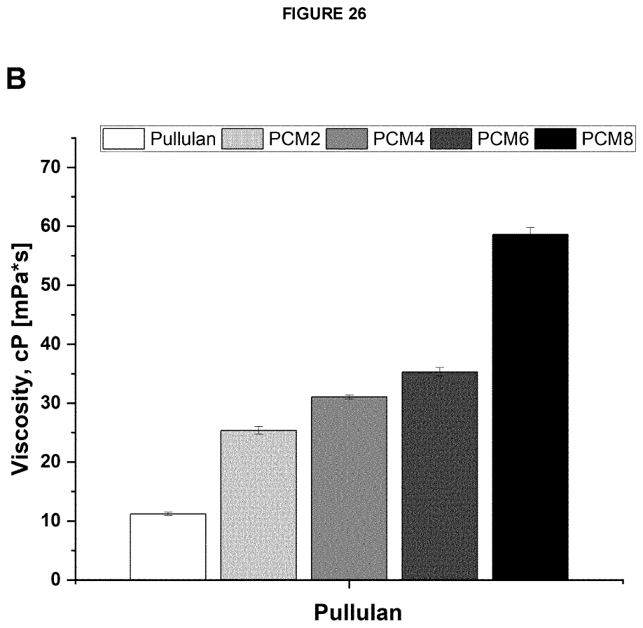

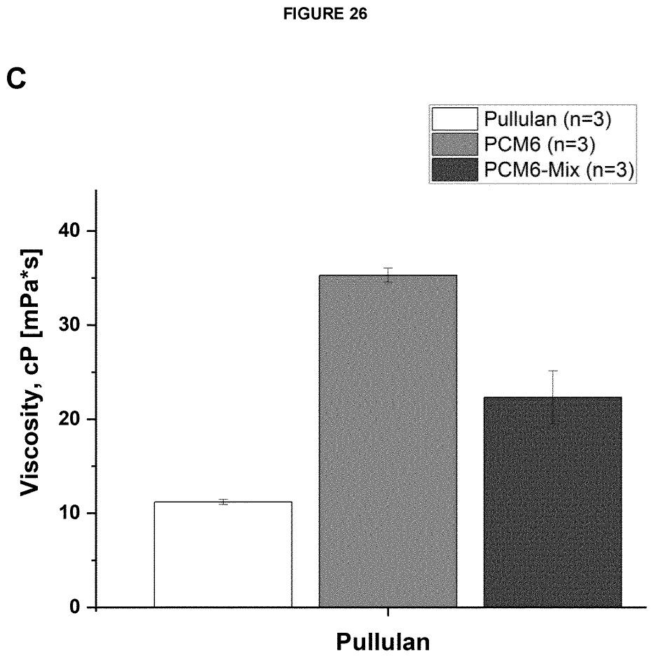

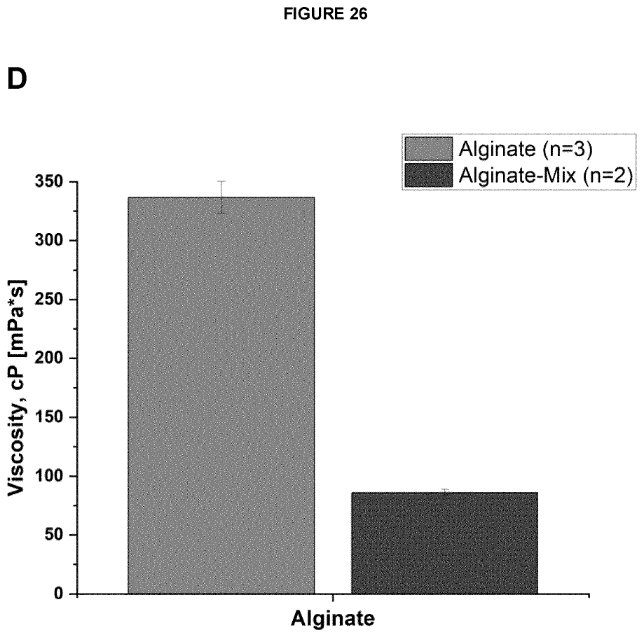

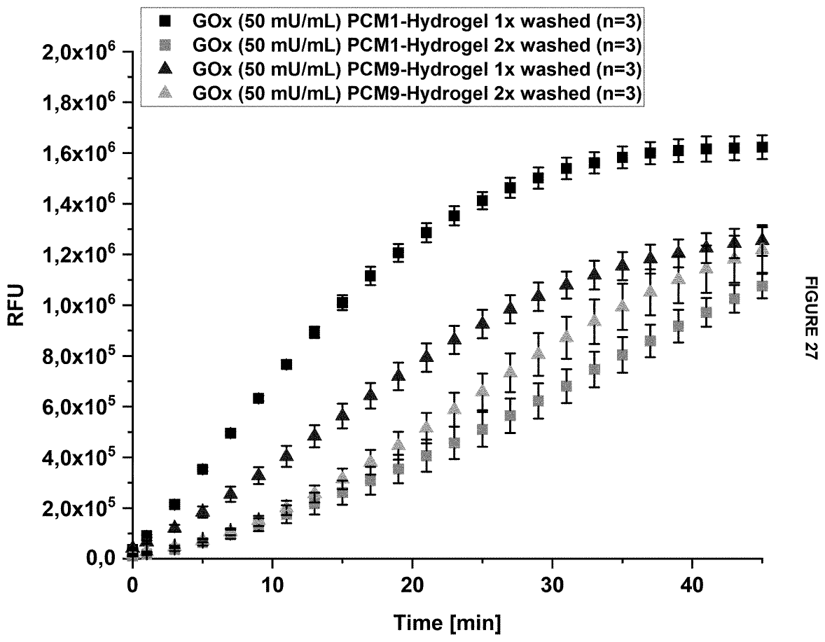

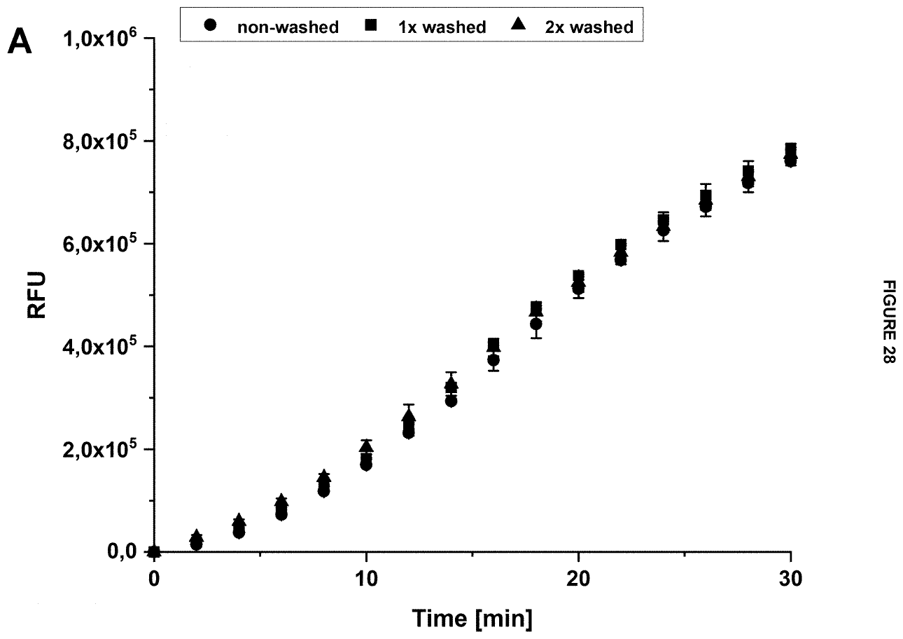

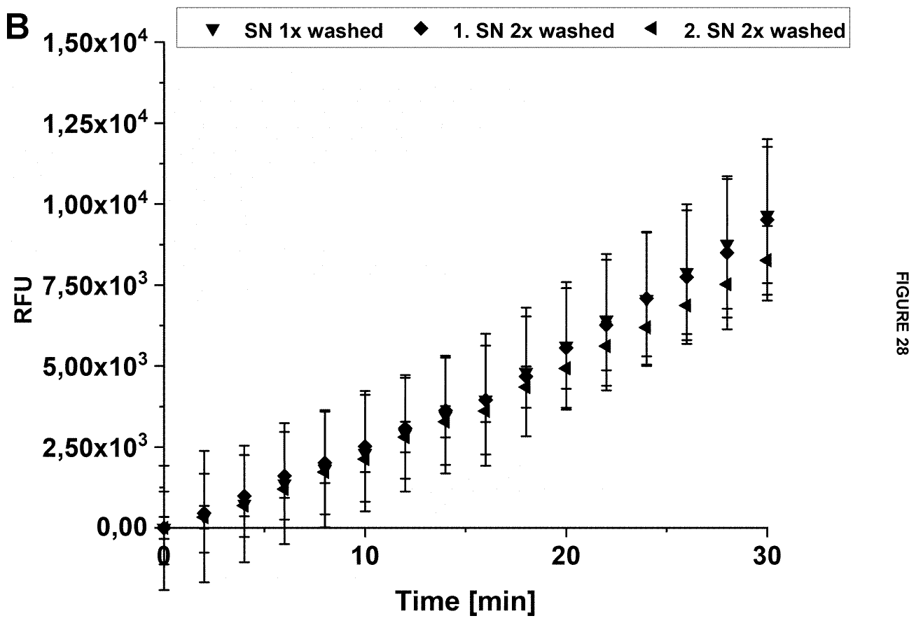

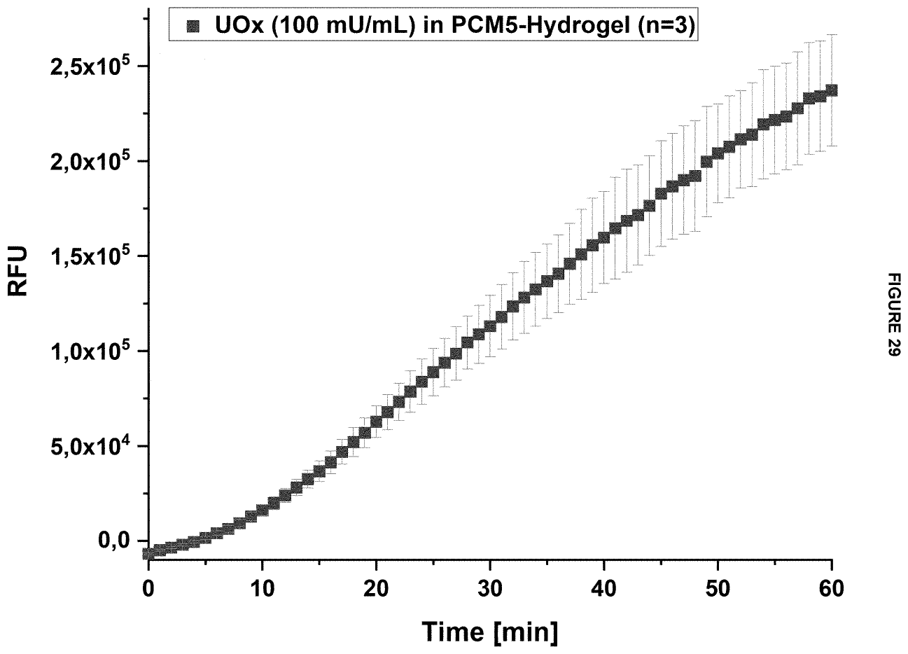

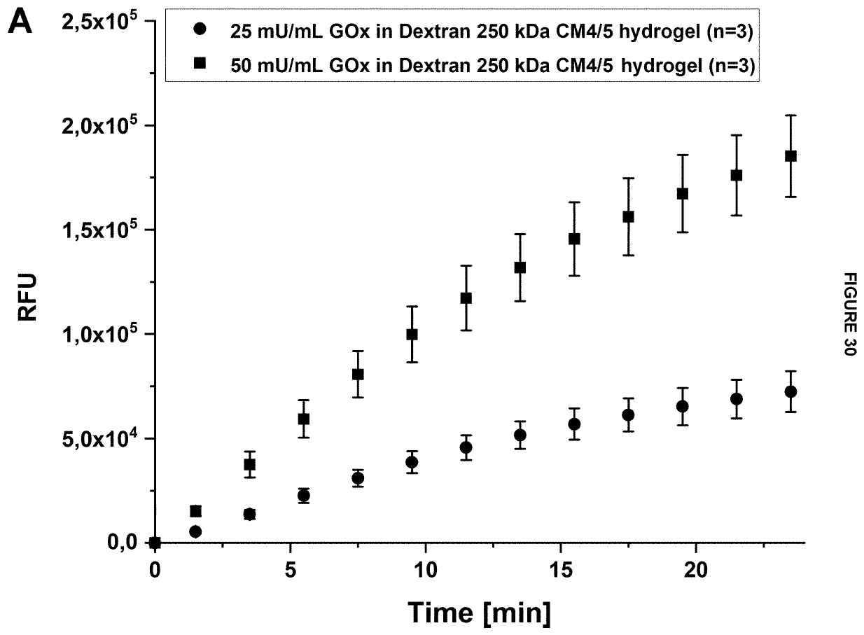

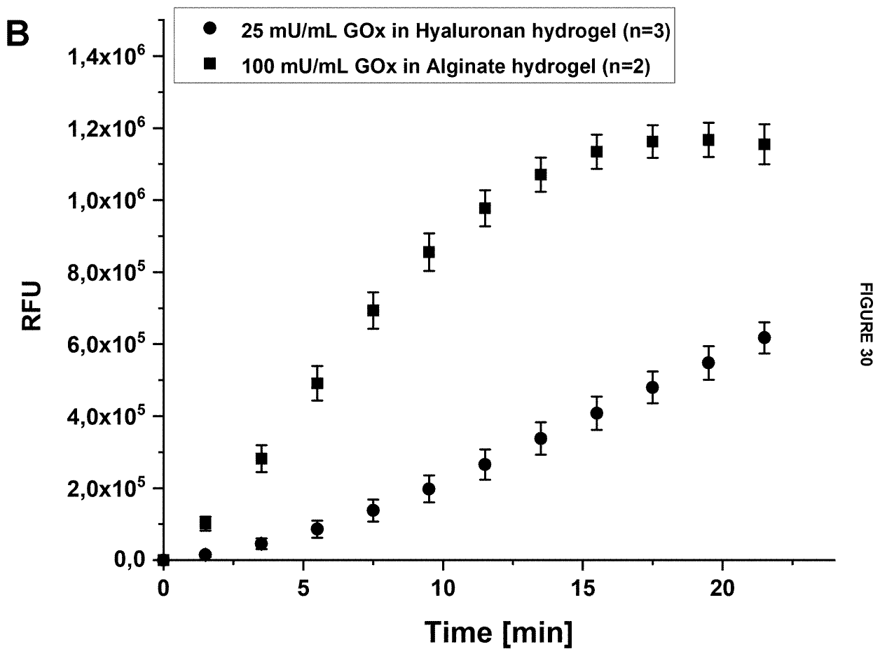

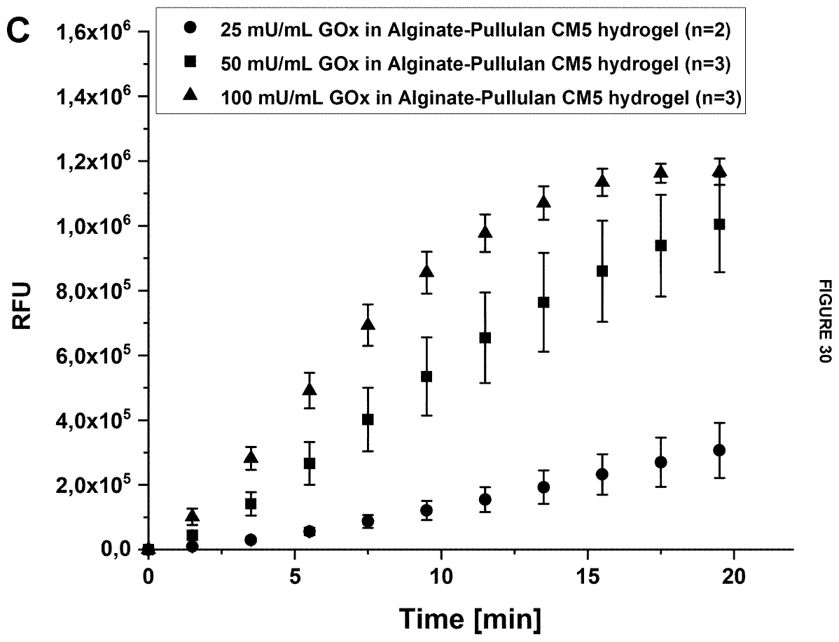

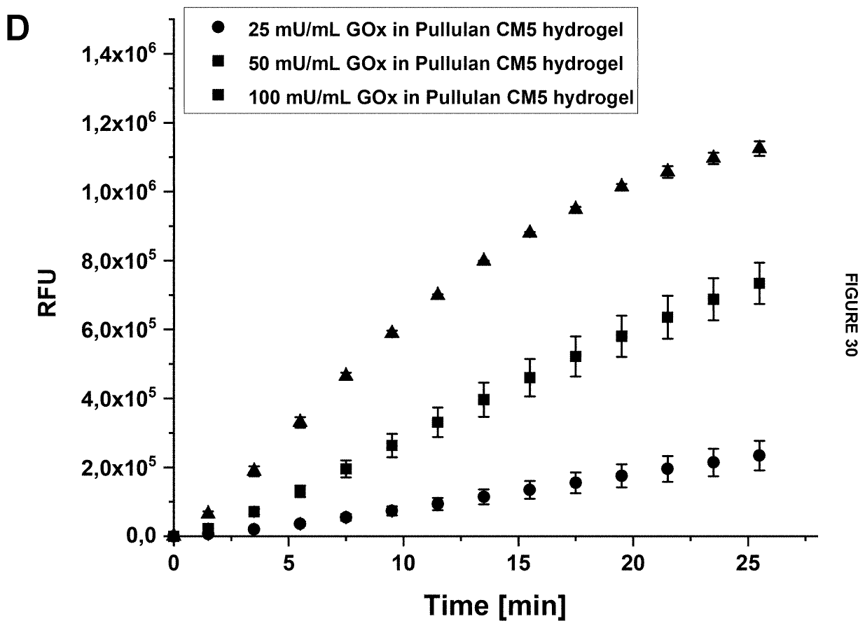

Long-term data of a low modified hydrogel (PCM1) with immobilized uricase were performed and the enzymatic activity was plotted over 22 days (FIG. 17).

The invention is further characterized by the following items:

### Items:

1. Method of preparing a biocompatible hydrogel, comprising the following steps:
a) Providing a first polysaccharide and a second polysaccharide, preferably wherein the first polysaccharide and/or the second polysaccharide being independently from each other selected from the group consisting of pullulan, alginate, hyaluronan and dextran, with n being the number of the monomeric repeating unit of the first and/or second polysaccharide and with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure —X, when the monomeric repeating unit of the first polysaccharide comprises a carboxylic acid residue,
   wherein A is —(CH₂)_{d}—C(O)— or —C(O)—NH—, wherein d is an integer from 1 to 3,
   wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃
   with r being an integer from 2 to 20,
   s being an integer from 1 to 15, and
   t being an integer from 1 to 15;
and
functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure —Y, when the monomeric repeating unit of the second polysaccharide comprises a carboxylic acid residue,
   wherein A′ is —(CH₂)_{d}—C(O)— or —C(O)—NH—, wherein d is an integer from 1 to 3,
   wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
   wherein Q is

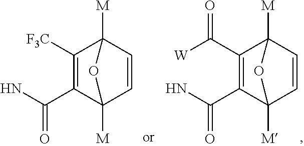

wherein M, M′=H or Me, and
wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

2. The method of preparing a biocompatible hydrogel according to item 1, wherein the first polysaccharide and the second polysaccharide are independently from each other selected from the group consisting of pullulan, alginate, cellulose, hyaluronan, dextran, lichenin, lentinan and mixtures thereof.

3. The method of preparing a biocompatible hydrogel according to item 1 or item 2, wherein the first polysaccharide and the second polysaccharide are independently from each other selected from the group consisting of pullulan, alginate, hyaluronan, dextran and mixtures thereof.

4. The method of preparing a biocompatible hydrogel according to any one of the preceding items, wherein the first polysaccharide and second polysaccharide are pullulan.

5. The method of preparing a biocompatible hydrogel according to any one of the preceding items, wherein the first and/or second polysaccharide is dextran or pullulan and carboxymethylation of at least one OH-group of dextran or pullulan is carried out in step b).

6. The method of preparing a biocompatible hydrogel according to any one of the preceding items, wherein in step c) the first polysaccharide is functionalized with 0.01-1.5 of A or X per monomeric repeating unit of the first polysaccharide.

7. The method of preparing a biocompatible hydrogel according to any one of the preceding items, wherein in step c) the second polysaccharide is functionalized with 0.01-1.5 of A′ or Y per monomeric repeating unit of the second polysaccharide.

8. The method of preparing a biocompatible hydrogel according to any one of the preceding items, wherein d is 1.

9. The method of preparing a biocompatible hydrogel according to any one of the preceding items, wherein step e) is a thermo-induced cycloaddition reaction between X and Y for forming a crosslinked polymer.

10. The method of preparing a biocompatible hydrogel according to any one of the preceding items, wherein the content of N₃ is 0.01-1.5 N₃ per monomeric repeating unit of the first polysaccharide.

11. The method of preparing a biocompatible hydrogel according to any one of the preceding items, wherein the content of Q is 0.01-1.5 per monomeric repeating unit of the first and/or second polysaccharide.

12. The method of preparing a biocompatible hydrogel according to any one of the preceding items, wherein in step c) -A-X is linked to at least one primary or secondary OH-group of the first polysaccharide, preferably via at least one of C₂, C₃, C₄ or C₆ of the monomeric repeating unit of the first polysaccharide, more preferably via C₆ of the monomeric repeating unit of the first polysaccharide.

13. The method of preparing a biocompatible hydrogel according to any one of the preceding items, wherein in step c) -A′-Y is linked to at least one primary or secondary OH-group of the second polysaccharide, preferably via at least one of C₂, C₃, C₄ or C₆ of the monomeric repeating unit of the second polysaccharide, more preferably via C₆ of the monomeric repeating unit of the second polysaccharide.

14. The method of preparing a biocompatible hydrogel according to any one of the preceding items, wherein the method is without the use of toxic reagents, preferably without the use of glutaraldehyde.

15. The method of preparing a biocompatible hydrogel according to any one of the preceding items, wherein the molecular weight of the unfunctionalized first polysaccharide is in the range from 5 to 2000 kDa.

16. The method of preparing a biocompatible hydrogel according to any one of the preceding items, wherein the molecular weight of the unfunctionalized second polysaccharide is in the range from 5 to 2000 kDa.

17. Hydrogel obtainable by a method of any one of the items 1 to 16.

18. The hydrogel according to item 17, wherein the hydrogel comprises one or more encapsulated enzyme(s).

19. The hydrogel according to item 17 or 18, wherein the hydrogel is a swellable or swollen hydrogel matrix.

20. A biocompatible hydrogel comprising:
a crosslinked polymer comprising the following structure:
   a first polysaccharide and a second polysaccharide,
      preferably wherein the first polysaccharide and/or the second polysaccharide being independently selected from the group consisting of pullulan, alginate, hyaluronan and dextran,
      with n being the number of the monomeric repeating unit of the first and/or second polysaccharide, and
      with n being an integer from 10 to 10000,
   one or more linker unit(s), which link the first polysaccharide with the second polysaccharide, wherein the structure of the one or more linker unit(s) is

-A-Z₁—B—Z₂-A′-,
wherein A and A′ are independently from each other —(CH₂)_{d}—C(O)— or —C(O)—NH—,
wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —OH, —(CH₂)ᵣ—, —NH—(CH₂CH₂O)ₛ—CH₂CH₂— and —NH—(CH₂—CH₂—C(O))ₜ—CH₂—CH₂—,
wherein B is

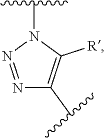

wherein R′ is selected from the group consisting of —CF₃, —C(O)—OMe, —C(O)—OEt, —C(O)—OH, —C(O)—NH₂ and —C(O)—NHMe,
for non-covalent immobilization of one or more enzyme(s).

21. The hydrogel according to item 20, wherein the hydrogel is a swellable or swollen hydrogel matrix.

22. The hydrogel according to item 20 or item 21, wherein the non-covalent immobilization of the one or more enzyme(s) comprises encapsulation of the one or more enzyme(s) and non-covalent binding of the one or more enzyme(s) in the hydrogel.

23. The hydrogel according to any one of items 20-22, wherein the first and/or second polysaccharide are independently from each other selected from the group consisting of pullulan, alginate, cellulose, hyaluronan, dextran, lichenin, lentinan and mixtures thereof.

24. The hydrogel according to any one of items 20-23, wherein the first and/or second polysaccharide is/are independently from each other selected from the group consisting of pullulan, alginate, hyaluronan, dextran, lichenin, lentinan and mixtures thereof.

25. The hydrogel according to any one of items 20-24, wherein the first and/or second polysaccharide is/are pullulan.

26. The hydrogel according to any one of items 20-25, wherein -A-Z₁— has the formula —CH₂—CO—NH—(CH₂—CH₂—O)ₙ—CH₂—CH₂—, wherein n is preferably from 1 to 5.

27. The hydrogel according to any one of items 20-26, wherein the first and/or the second polysaccharide has/have a concentration of 5-120 mg/ml, preferably 10-80 mg/ml, more preferably 20-60 mg/ml, with respect to the total hydrogel.

28. The hydrogel according to any one of items 20-27, wherein the one or more enzyme(s) for non-covalent immobilization is selected from the group consisting of lipases or oxidases, preferably glucose oxidase, lactate oxidase, uricase, glutamate oxidase, cortisol oxidase, xanthine oxidase, cholesterol oxidase, sarcosine oxidase, and alcohol oxidase.

29. A composition comprising the hydrogel according to any one of items 17-28.

30. Method for encapsulating one or more enzyme(s) in a hydrogel according to any one of items 17 to 28.

31. Use of a hydrogel according to any one of items 17 to 28
a) for non-covalent immobilization of one or more enzyme(s) in the hydrogel, or
b) in a biosensor.

32. A kit comprising the composition or the hydrogel according to any one of items 17 to 29.

### REFERENCES

1. Weikang Hu, Zijian Wang, Yu Xiao, Shengmin Zhanga and Jianglin Wang: Advances in crosslinking strategies of biomedical hydrogels; Biomaterial Science, 2019, 7, 843.
2. Yu F., X. Cao, J. Du, G. Wang and X. Chen, ACS Appl. Mater. Interfaces, 2015, 7, 24023-24031.
3. Zhang Y., L. Tao, S. Li and Y. Wei, Biomacromolecules, 2011, 12, 2894-2901. doi: 10.1021/bm200423f.
4. Ma X., T. Xu, W. Chen, H. Qin, B. Chi and Z. Ye, Carbohydr. Polym., 2018, 179, 100-109. doi: 10.1016/j.carbpol.2017.09.071.
5. Lin F., J. Yu, W. Tang, J. Zheng, A. Defante, K. Guo, C. Wesdemiotis and M. L. Becker, Biomacromolecules, 2013, 14, 3749-3758. doi: 10.1021/bm401133r.
6. Peng Yong Y., Veronica Glattauer, and John A. M. Ramshaw: Research Article Stabilisation of Collagen Sponges by Glutaraldehyde Vapour Crosslinking, Hindawi, International Journal of Biomaterials, Volume 2017, Article ID 8947823, 6 pages, doi:10.1155/2017/8947823.
7. Jia W., A. J. Bandodkar, G. Valdes-Ramirez, J. R. Windmiller, Z. Yang, Ramirez, G. Chan, Wang, Anal. Chem., 2013, 85, 6553.
8. Lopez-Gallego F, Betancor L, Mateo C, Hidalgo A, Alonso-Morales N, Dellamora-Ortiz G, Guisan J M, Fernandez-Lafuente R. Enzyme stabilization by glutaraldehyde crosslinking of adsorbed proteins on aminated supports. J Biotechnol., 2005; 119(1):70-5.

1. A method of preparing a biocompatible hydrogel,
comprising the following steps:
a) Providing a first polysaccharide and a second polysaccharide,
optionally wherein the first polysaccharide and/or the second polysaccharide being independently from each other selected from the group consisting of pullulan, alginate, cellulose, hyaluronan, dextran, lichenin, lentinan and mixtures thereof,
with n being the number of the monomeric repeating unit of the first and/or second polysaccharide and with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide does not comprise a carboxylic acid residue, or functionalization of the first polysaccharide with one or more linker unit(s) of the structure —X, when the monomeric repeating unit of the first polysaccharide comprises a carboxylic acid residue,
wherein A is —(CH₂)_{d}—C(O)— or —C(O)—NH—, wherein d is an integer from 1 to 3,
wherein X is selected from the group consisting of —NH—(CH₂)ᵣN₃, —NH—(CH₂CH₂O)ₛ—CH₂CH₂N₃, and —NH—(CH₂—CH₂—C(O))ₜ—CH₂—CH₂—N₃,
with r being an integer from 2 to 20,
s being an integer from 1 to 15, and
t being an integer from 1 to 15;
and
functionalization of the second polysaccharide with one or more linker unit(s) of the structure -A′-Y, when the monomeric repeating unit of the second polysaccharide does not comprise a carboxylic acid residue, or functionalization of the second polysaccharide with one or more linker unit(s) of the structure —Y, when the monomeric repeating unit of the second polysaccharide comprises a carboxylic acid residue,
wherein A′ is —(CH₂)_{d}—C(O)— or —C(O)—NH—, wherein d is an integer from 1 to 3,
wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,
wherein Q is 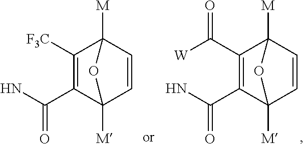
wherein M, M′=H or Me, and
wherein W=OMe, OEt, OH, NH₂ or NHMe,
d) optionally having the addition of one or more enzyme(s) to the mixture formed by steps a)-c), and
e) incubation of the mixture formed by steps a)-d) in an aqueous medium at a temperature being in one of the range from 25° C. to 70° C., or at 40° C., for one of at least 1 hour, or for 1 to 10 hours.

2. The method of preparing a biocompatible hydrogel according to claim 1,
wherein (a) the first polysaccharide and the second polysaccharide are independently from each other selected from the group consisting of pullulan, alginate, cellulose, hyaluronan, dextran, lichenin, lentinan and mixtures thereof, or (b) wherein the first polysaccharide and the second polysaccharide are independently from each other selected from the group consisting of pullulan, alginate, hyaluronan, dextran and mixtures thereof, or (c) wherein the first polysaccharide and second polysaccharide are pullulan.

3. The method of preparing a biocompatible hydrogel according to claim 1, wherein the first and/or second polysaccharide is dextran or pullulan and carboxymethylation of at least one OH-group of dextran or pullulan is carried out in step b).

4. The method of preparing a biocompatible hydrogel according to claim 1, wherein in step c) the first polysaccharide is functionalized with 0.01-1.5 of A or X per monomeric repeating unit of the first polysaccharide and/or wherein in step c) the second polysaccharide is functionalized with 0.01-1.5 of A′ or Y per monomeric repeating unit of the second polysaccharide.

5. The method of preparing a biocompatible hydrogel according to claim 1, wherein step e) is a thermo-induced cycloaddition reaction between X and Y for forming a crosslinked polymer.

6. The method of preparing a biocompatible hydrogel according to claim 1, wherein the content of N₃ is 0.01-1.5 N₃ per monomeric repeating unit of the first polysaccharide and/or wherein the content of Q is 0.01-1.5 per monomeric repeating unit of the first and/or second polysaccharide.

7. The method of preparing a biocompatible hydrogel according to claim 1,
wherein in step c) -A-X is linked to at least one primary or secondary OH-group of the first polysaccharide, or via at least one of C₂, C₃, C₄ or C₆ of the monomeric repeating unit of the first polysaccharide, or via C₆ of the monomeric repeating unit of the first polysaccharide and/or
wherein in step c) -A′-Y is linked to at least one primary or secondary OH-group of the second polysaccharide, or via at least one of C₂, C₃, C₄ or C₆ of the monomeric repeating unit of the second polysaccharide, or via C₆ of the monomeric repeating unit of the second polysaccharide.

8. The method of preparing a biocompatible hydrogel according to claim 1, wherein the molecular weight of the unfunctionalized first polysaccharide is in the range from 5 to 2000 kDa and/or wherein the molecular weight of the unfunctionalized second polysaccharide is in the range from 5 to 2000 kDa.

9. A hydrogel obtainable by a method of claim 1, optionally wherein the hydrogel comprises one or more encapsulated enzyme(s).

10. A biocompatible hydrogel comprising:
a crosslinked polymer comprising the following structure:
a first polysaccharide and a second polysaccharide,
optionally wherein the first polysaccharide and/or the second polysaccharide being independently selected from the group consisting of pullulan, alginate, cellulose, hyaluronan, dextran, lichenin, lentinan and mixtures thereof,
with n being the number of the monomeric repeating unit of the first and/or second polysaccharide, and
with n being an integer from 10 to 10000,
one or more linker unit(s), which link the first polysaccharide with the second polysaccharide, wherein the structure of the one or more linker unit(s) is
-A-Z₁—B—Z₂-A′-,
wherein A and A′ are independently from each other —(CH₂)_{d}—C(O)— or —C(O)—NH—,
wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—,
with
d being an integer from 1 to 4,
r being an integer from 2 to 20,
s being an integer from 1 to 15, and
t being an integer from 1 to 15;
and,
wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,
wherein B is 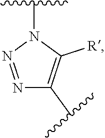
and
wherein R′ is selected from the group consisting of —CF₃, —C(O)—OMe, —C(O)—OEt, —C(O)—OH, —C(O)—NH₂ and —C(O)—NHMe, for non-covalent immobilization of one or more enzyme(s).

11. The hydrogel according to claim 10, wherein the non-covalent immobilization of the one or more enzyme(s) comprises encapsulation of the one or more enzyme(s) and non-covalent binding of the one or more enzyme(s) in the hydrogel.

12. The hydrogel according to claim 10, wherein the first and/or second polysaccharide is/are independently from each other selected from the group consisting of pullulan, alginate, cellulose, hyaluronan, dextran, lichenin, lentinan and mixtures thereof and/or either wherein the first and/or second polysaccharide is/are independently from each other selected from the group consisting of pullulan, alginate, hyaluronan, dextran, lichenin, lentinan and mixtures thereof, or wherein the first and/or second polysaccharide is/are pullulan.

13. The hydrogel according to claim 10, wherein -A-Z₁— has the formula —CH₂—CO—NH—(CH₂—CH₂—O)ₙ—CH₂—CH₂—, wherein n is optionally from 1 to 5 and/or wherein the first and/or the second polysaccharide has/have a concentration of one of 5-120 mg/ml, 10-80 mg/ml, or 20-60 mg/ml, with respect to the total hydrogel and/or wherein the enzyme for non-covalent immobilization is selected from the group consisting of lipases and oxidases, or of the group consisting of glucose oxidase, lactate oxidase, uricase, glutamate oxidase, cortisol oxidase, xanthine oxidase, cholesterol oxidase, sarcosine oxidase, and alcohol oxidase.

14. A composition comprising the hydrogel according to claim 9.

15. A method for encapsulating one or more enzyme(s) in a hydrogel according to claim 9.

16. A composition comprising the hydrogel according to claim 10.

17. A method for encapsulating one or more enzyme(s) in a hydrogel according to claim 10.

18. The method according to claim 1, wherein the first polysaccharide and/or the second polysaccharide being independently from each other selected from the group consisting of pullulan, alginate, cellulose, hyaluronan, dextran, lichenin, lentinan and mixtures thereof.

19. The hydrogel according to claim 9, wherein the hydrogel comprises one or more encapsulated enzyme(s).

20. Biosensor comprising the hydrogel according to claim 10.
